(19) United States
(12) Patent Application Publication
(10) Pub. No.: US 2012/0149888 A1
(43) Pub. Date: Jun. 14, 2012

(54) SYNTHESIS OF ARA-2'-O-METHYL-NUCLEOSIDES, CORRESPONDING PHOSPHORAMIDITES AND OLIGONUCLEOTIDES INCORPORATING NOVEL MODIFICATIONS FOR BIOLOGICAL APPLICATION IN THERAPEUCTICS, DIAGNOSTICS, G- TETRAD FORMING OLIGONUCLEOTIDES AND APTAMERS

(76) Inventors: Suresh C. Srivastava, Burlington, MA (US); Divya Pandey, Lucknow (IN); Naveen P. Srivastava, Burlington, MA (US); Alok Srivastava, Burlington, MA (US)

(21) Appl. No.: 13/138,465

(22) PCT Filed: Feb. 23, 2010
(86) PCT No.: PCT/US2010/000524

§ 371 Date: Nov. 7, 2011

Related U.S. Application Data
(60) Provisional application No. 61/208,287, filed on Feb. 22, 2009.

(51) Int. Cl. C07H 21/00; C07H 19/09; C07H 19/19

(52) U.S. Cl. 536/23.1; 536/27.4; 536/28.53; 536/28.5; 536/28.51; 536/26.7; 536/26.8

(57) ABSTRACT

The present invention relates to synthesis, purification and methods to obtain high purity novel 2′-arabino-O-methyl nucleosides and the corresponding phosphoramidites of various arabinonucleoside bases and introduction of such units into defined sequence synthetic DNA and RNA. Various synthetic oligonucleotides, such as HIV integrase inhibitor 14-mer and thrombin binding oligonucleotide, thrombin-1, bearing ara-2′-omethyl modification have been synthesized. It is anticipated the oligonucleotides incorporating these monomers will exhibit biological activities related to antisense approach approach, design of better SiRNA's, diagnostic agents. Similarly, it is anticipated that oligonucleotides incorporating such novel nucleosides will be useful to develop therapeutic candidates designing stable G-quadruplexes and Aptamers for oligonucleotide structure, folding topology, evaluation of biochemical properties and design and develop as therapeutic agents. It is further anticipated that the nucleosides, phosphates and triphosphates of this invention could develop as therapeutic agents.

### CROSS-REFERENCE TO RELATED APPLICATIONS

This application claims priority from U.S. provisional patent application Ser. No. 61/208287, filed by the inventors on Feb. 22, 2009. The entire contents of the prior application are herein incorporated by reference.

### FIELD OF THE INVENTION

The present invention relates to the synthesis, purification and methods to obtain high purity, novel 2′-arabino-O-methyl nucleosides of various arabinonucleoside bases and to the introduction of such units into defined sequence synthetic DNA and RNA. The oligonucleotides incorporating these monomers may lead to the design of better SiRNA's, diagnostic agents and be useful to develop therapeutic candidates incorporating stable G-quadruplexes and Aptamers for oligonucleotide structure.

### BACKGROUND OF THE INVENTION

Various major class of oligonucleotides pertinent to therapeutic and diagnostics applications, which have great promise for therapeutic application, are antisense (i.e. sequences complementary to the “sense” strand, usually the messenger RNA and otherwise interfering (e.g. “decoy”) oligonucleotides (collectively referred to herein as ASO's); have gained overwhelming popularity for interference in various steps leading from DNA translation. This regulatory interference can be harnessed for therapeutic effects against many diseases & viral infections. Such sequences are complimentary to portions of mRNA for that protein (i.e. antisense). Other regulatory mechanisms of viruses, such as HIV, are also open to interference with the use of complimentary (i.e. antisense) or decoy (i.e. sense) oligonucleotides and the oligonucleotides for extremely high specificity and the case of design afforded by Watson-crick base pairing. The high association constants imply a strong duplex formation and thus effectiveness at low concentration. In the design of ASO one of the important criteria is stability towards degradation in vivo, retention inside cells, devoid of non-specific interaction with other cellular factors, have low toxicity, non-immunogenic, non-mutagenic, and should have a large therapeutic window. Ideally should possess RNaseH activity upon binding its target and will have a significant advantage as antisense to mRNA, destroying the mRNA, and releasing the ASO and potentially creating a catalytic cycle. This is especially important in case of many viral infections, such as HIV, since the reverse transcriptase; which is localized in the cytoplasm itself, has RNaseH activity. Cellular RNaseH has been thought to be localized primarily in the nucleus and to some extent in the cytoplasm as well. Natural phosphodiester ASOs are subject to nuclease activity and therefore possess a short half life.

Modification of the 2′-Ara-OH group with 2′-fluoro- was reported recently. The 2′-deoxy-2′-fluoroD-arabino nucleic acid analogs (FIG. 1), commonly abbreviated as 2′-F-ANA (C. J. Wilds and M. J. Damha, Nucleic Acids. Res., 28, 18, 3625-3635, 2000; C. G. Peng and M. J. Damha, Nucl. Acids Res., 35, 15, 4977-4988, 1997), have interesting physico-chemical and biological properties and result in stabilization of G-quadruplex. Further the 2′-F-ANA has enhanced RNA affinity relative to that of DNA and phosphorothioate DNA. The 2′-F-ANA had favorable base pairing to single stranded DNA also. The oligonucleotides derived from the 2′-F-ANA units, were found to be substrates of RNaseH. This was postulated to be due to the “near deoxy structure” of 2-F-ANA units. The oligonucleotides derived from Arabinonucleic acid, commonly abbreviated as ANA have been shown to form hybrid with RNA. They have also been shown to be substrates of RNaseH. The difference between ANA and RNA results from the configurational difference, wherein the ANA 2′-hydroxyl is cis (structure 2) with respect to the heterocyclic base. Although DNA and RNA do form hybrid, but the duplex stability is less than natural DNA/RNA hybrids or s-DNA/RNA hybrids. In regard to G-quadruplex formation it has been shown that replacement of deoxy Guanosine with 2′-ara-fluoro-2′-deoxyguanosine

(FANA) adapts anti conformation (structure 1) when this nucleoside is incorporated in oligo sequences rich in G. While 2′-Hydroxyl group in ara guanosine adopts a syn conformation (structure 2). FANA sequences were shown to stabilize G-quartets and maintain the quadruplex conformation. 2′-F-ANA based oligo nucleotides have been also directed towards developing improved SiRNA (Dowler T., Bergeron D., Tedeschi A. L., Pacqet L., Ferrari N., Damha M. J., Nucl. Acids Res., 34: 1669-1675, 2006.

It has been shown that the syn and anti glycosidic bond configuration determines the folding topology in various G-quadruplex oligonucleotides. The guanosine residues can exist as either syn or anti conformation, and the deoxy-D-ribose puckering could be either endo or exo (structure 3 & 4) respectively. The guanosine residues in the cross over basket form of Oxy28 of Oxytrichia trifalax telomeric DNA sequence are alternatively syn-anti-syn along the G-4 track, and each sugar is in the 2′-endo conformation (Blackburn, E. H., J. Biol. Chem. 265, 5919-5921, 1990).

It has been shown that the G-quadruplexes in telomeres can exist in various forms, based on folding topology of G-tracks within a strand of DNA or between two strands of DNA's. They can exist as parallel (structure 5) or antiparallel (structure 6) strand. In the human telomeric DNA sequences all the Guanosine residues are anti in the intramolecular parallel G-quadruplexes (Smith, F. W., Schultze, P., Feigon, J. Structure 3, 997-1008, 2000).

Since the sugar when constrained in the 3′-endo conformation forces the glycosidic bond conformation to be in anti conformation (structure 3). Therefore we hypothesized that 2′-OMethyl-ara-guanosine residues in oligonucleotides would force the sugar in a rigid 2′-endo conformation (south/east) and the Guanosine units will have strong steric repulsion of bases with the β face of 2′-Ara-O-methyl group. Even other nucleoside bases such as adenosine, cytosine, uracil would prefer the anti conformation (structure 5). With Ara-G containing sequences we expect strong locking of conformations. Further it is very clear that the design of locked conformations to develop selective aptamers is very possible.

The G-rich quadruplex sequences are present in the G-tetrad sequences of telomeres. Telomeres are specialized DNA structures at the end of chromosomes, complexed with proteins. The DNA within telomeres are rich in highly repetitive G-rich quadruplexes, and they are responsible for many key biochemical processes (Williamson, J. R., Ann. Rev. Biophys. Biomol. Struct., 23, 703-730, 1994; Williamson, J. R. Raghuraman, M. K. and Cech, T. R., Cell, 59, 871-880, 1989; Smith, F. W. and Feigon, J., Nature, 356, 164-168, 1992; Borman S. Targeting telomerase. Chem. Eng. News, 84: 32-33, 2006; Mcaya, R. F., Schultz, P., Smith, F. W., Roe, J. A., and Feigon, J Proc. Nal. Acad. Sci. USA 90, 3745-3749, 1993; Mazumdar, A., Neamati., N., Ojwang, J. O., Sunder, S., Rando, R. F., and Pommier, Y., Biochemistry, 35, 13762-13771, 1996). G rich sequences which are widely spread in telomerase and are responsible for many key biochemical processes have been subject of intense research. The exceptional stability of G quadruplexes and the topology of the three dimensional structure has been widely studied and efforts have been made to modify bases so that DNA with stable G-quadruplex can be synthesized. The 2′-deoxy-2′-fluoro-arabinonucleic acid (2′-F-ANA) containing oligonucleotides, as eluded in the proceeding paragraph help to stabilize oligonucleotide G-quartets and form stable G quadruplex.

It has been shown that G-quadruplex formation, which consist of natural G bases require facilitation of telomerase proteins, and the natural G bases alone are not sufficient for G-based quadruplexes. The hyperactivity of telomerase results in many forms of cancer. Systematic efforts on the design of stable G quartet, which are stable in forming oligonucleotides have been on going (Wyatt, J. R., Vickers, T. A., Roberson, J. L. and Buckheit, R. W., Klimkait, T., Debaets, F, Davis, P. W., Rayner, B., Imbach, J. L and Ecker, D. J., Proc. Natl. Acad. Sci. USA 91, 1356-1360, 1994; Jing, N. J., and Hogan, M. E., J. Biol. Chem., 273, 34992-34999, 1998; Jing, N., Rando, R. F., Pommier, Y., and Hogan, M. E., Biochemistry, 36, 12498-12505, 1997; T. Kuwasaki, M. Hatta, H. Takeuchi, and H. Takaku, J. Antimicrob. Chemother., 51(4): 813-819, 2003). A number of oligonucleotides containing only G and T bases, which are capable of forming G-tetrad were found to be potent inhibitors of human immunodeficiency virus type 1 (HIV-1) replication in cell culture (Phan A. T., Kuryavyi, Ma J. B., Faure A., Andreola M. L, Patel D. J., Proc. Natl. Acad. Sci. USA, 102: 634-639, 2005; N. Jing, Y. Li, Xiong, W., Sha, W., Jing, L., Tweardy, D. J., Cancer Research, 64(18): 6603-6609, 2004).

Inhibition of human immunodeficiency virus 1 replication in vitro was observed by a self stabilized oligonucleotide with 2′-Omethyl guanosine-uridine quadruplex motifs. An interlocked dimeric parallel-stranded DNA quadruplex was found to be a potent inhibitor of HIV-1 integrase, (Siddiqi-Jain A., Grand C. L., Bearss D. J., Hurley, L. H., Proc. Natl. Acad. Sci. USA, 99, 11593-11598, 2002).

G-Quadruplex oligonucleotides were found to be involved in signal transduction and activation in the growth of Prostrate and Breast cancer by transcription inhibition and apoptosis (N. Jing, Y. Li, Xiong, W., Sha, W., Jing, L., Tweardy, D. J., Cancer Research, 64(18): 6603-6609, 2004). It has been shown that G-quadruplex are present in promoter region, which can be targeted with small molecules to repress c-MYC transcription (Siddiqi-Jain A., Grand C. L., Bearss D. J., Hurley, L. H., Proc. Natl. Acad. Sci. USA, 99, 11593-11598, 2002)

Inhibition of human immunodeficiency virus type 1 activity in vitro was demonstrated by a self stabilized oligonucleotide with guanosine-thymidine quadruplex motif was demonstrated (J.-I. Suzuki, Miyno-Kurosaki N., Kuwasaki, T., Takeuchi, G., Kawai, G., Takaku, H., J. Virol., 76(6), 3015-3022, 2002).

A comprehensive review of structure-activity relationship of a family of G-tetrad forming oligonucleotides as potent HIV inhibitors has been carried out, which outlines basis for anti-HIV drug design (N. Jing, De Clerque, E., Rando, R. F., Pallansch, L., Lackman-Smith, C., Lee, S., Hogan, M. E., Biol. Chem., 275(5), 3421-3430, 2000). Several G-quadruplex containing aptameters have been found to inhibit cell transfection by HIV in vitro G-Quadruplex containing aptamers bind and inhibit thrombin, a key enzyme in blood clotting (Macaya, R. F., Schultz, P., Smith, F. W., Roe, J. A and Feigon J., Proc. Nat Acad. Sci. USA, 90, 3745-3749, 1993; Wang, K. Y., McCurdy, S. N., Shea, R. G., Swaminathan, S., and Bolton, P. H., Biochemistry, 32, 1899-1904, 1993). Thrombin is a key enzyme involved in blood clotting cascade. Studies have been carried out on the effect of chemical modifications on the thermal stability of different G-quadruplex forming oligonucleotides have been on going (Sacca B., Lacroix L., Mergny J-L., Nucl. Acids Res., 33: 1182-1192, 2005). Cis-modifications such as phosphorothioate (S), p-methylphosphonate, 2′-O methyl (2′-3′-diol system) analogs were studied and it could be concluded that such studies provide useful information for the modulation of G-quadruplex to develop therapeutically useful oligonucleotides and to determine structural elements of G-quadruplexes in order to get better insight into factors regulating their formation and stabilization. A conformationally constrained nucleotide analogue has been shown to control the folding topology of a G-quadruplex derived from deoxy guanosine containing J. A. Rottman, F. and Heinlein, K., Biochemistry, 7, 2634-2641, 1968).

The following prior art references, some of which are cited above, summarize the present state of the art :
(i) Smith, F. W., Schultze, P., Feigon, J. Structure 3, 997-1008, 2000
(ii) Keniry, M. A. Biopolymers 2000, 56, 123-146
(iii) Blackburn, E. H., J. Biol. Chem. 265, 5919-5921, 1990
(iv) Blackburn, E. H., Nature, 350, 569-573, 1991;
(v) Zachian, V. A. Annu. Rev. Genet., 23, 579-604, 1989
(vi) Williamson, J. R., Ann. Rev. Biophys. Biomol. Struct., 23, 703-730, 1994
(vii) Williamson, J. R. Raghuraman, M. K. and Cech, T. R., Cell, 59, 871-880, 1989
(viii) Smith, F. W. and Feigon, J., Nature, 356, 164-168, 1992
(ix) Borman S. Targeting telomerase. Chem. Eng. News, 84: 32-33, 2006
(x) Mcaya, R. F., Schultz, P., Smith, F. W., Roe, J. A., and Feigon, J Proc. Nal. Acad. Sci. USA 90, 3745-3749, 1993
(xi) Mazumdar, A., Neamati., N., Ojwang, J. O., Sunder, S., Rando, R. F., and Pommier, Y., Biochemistry, 35, 13762-13771, 1996
(xii) Wyatt, J. R., Vickers, T. A., Roberson, J. L. and Buckheit, R. W., Klimkait, T., Debaets, F, Davis, P. W., Rayner, B., Imbach, J. L and Ecker, D. J., Proc. Natl. Acad. Sci. USA 91, 1356-1360, 1994
(xiii) Jing, N. J., and Hogan, M. E., J. Biol. Chem., 273, 34992-34999, 1998
(xiv) Jing, N., Rando, R. F., Pommier, Y., and Hogan, M. E., Biochemistry, 36, 12498-12505, 1997
(xv) T. Kuwasaki, M. Hatta, H. Takeuchi, and H. Takaku, J. Antimicrob. Chemother., 51(4): 813-819, 2003
(xvi) Phan A. T., Kuryavyi, Ma J. B., Faure A., Andreola M. L, Patel D. J., Proc. Natl. Acad. Sci. USA, 102: 634-639, 2005
(xvii) N. Jing, Y. Li, Xiong, W., Sha, W., Jing, L., Tweardy, D. J., Cancer Research, 64(18): 6603-6609, 2004
(xviii) Siddiqi-Jain A., Grand C. L., Bearss D. J., Hurley, L. H., Proc. Natl. Acad. Sci. USA, 99, 11593-11598, 2002
(xix) J.-i. Suzuki, Miyno-Kurosaki N., Kuwasaki, T., Takeuchi, G., Kawai, G., Takaku, H., J. Virol., 76(6), 3015-3022, 2002
(xx) N. Jing, De Clerque, E., Rando, R. F., Pallansch, L., Lackman-Smith, C., Lee, S., Hogan, M. E., Biol. Chem., 275(5), 3421-3430, 2000
(xxi) Macaya, R. F., Schultz, P., Smith, F. W., Roe, J. A and Feigon J., Proc. Nat Acad. Sci. USA, 90, 3745-3749, 1993
(xxii) Wang, K. Y., McCurdy, S. N., Shea, R. G., Swaminathan, S., and Bolton, P. H., Biochemistry, 32, 1899-1904, 1993
(xxiii) Sacca B., Lacroix L., Mergny J-L., Nucl. Acids Res., 33: 1182-1192, 2005
(xxiv) J. Am Rottman, F. and Heinlein, K (1968) Biochemistry 7, 2634-2641).
(xxv) Hideo Inoue, Yoji Hayase, Akihiro Imura, Shigenori Iwai, Kazunobu Miura and Eiko Ohtsuka (1987). Soc., 126: 500-5051, 2004.

### Ara-(2′)-Omethyl-β-D-nucleoside Phosphoramidites & Triphosphates & Solid Supports

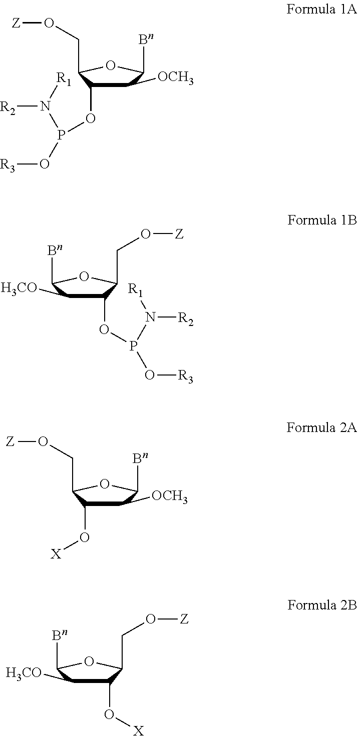

The formula 2A and 2B represent Z; H and X; H ; B is natural nucleobases, adenine, guanine, cytosine, uracil, thymine or any of the modified nucleosides, optionally unprotected. The formula 2A represents ara-(2′)-Omethyl-beta-D nucleoside and formula 2B represents ara -(2′)-Omethyl-beta-L-nucleosides (the mirror image). The formula 2A and 2B further represent; Z & X as monophosphate, diphosphate, or triphosphate alternatively at either the Z or X position, in combination of Z and X beung H alternatively.

### Ara-(2′)-Omethyl-β-D-nucleoside Phosphoramidites & Triphosphates

Novel Modification for design of Highly Stable G-Quadruplex oligos & Aptamers G quadruplexes. It is expected that could lead to a very significant role in telomere DNA in chromosomes and may possess enormous potentials in many areas such as;

### Ara-(2′)-Omethyl-β-D-nucleoside Triphosphates Formula 2 A; Z; is triphosphate and X; H)

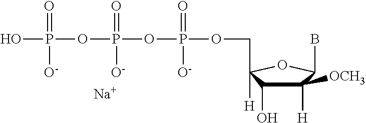

### Structure 2: Ara-2′-Omethyl-B-D-nucleoside-5′-triphosphates

B; Adenine
Cytosine
Guanine
Uracil or modified nucleo bases

Ara-2′-Omethyl-β-D-nucleoside Phosphoramidites & Triphosphates: The phosphoramidites are represented by formula 1A and 1B; where Z is typically a DMT (dimethoxytriphenyl) group and R3 is a phosphate cleaving group, generally 2-cyanoethyl Group. The formula 1B represents beta-L-conformation of the nucleoside (generally referred as mirror image).

### Ara-2′-Omethyl-β-D-nucleoside Phosphoramidites & Triphosphates:

### Ara-2′-O-Methyl-RNA Phosphoramidites

Methylation of ara nucleosides results in new class of modified nucleosides. The phosphoramidites of such modified nucleosides are expected to result in new class of oligos and there is possibility of many important class of oligonucleotides such as for design of highly Stable G-Quadruplex oligos & Aptamers.

G quadruplexes play a very significant role in telomere DNA in chromosomes with enormous potentials in many areas such as;
Cancer Therapy¹
HIV Inhibitors²⁻⁴
Conformational stability control of oligonucleotides⁵⁻⁸
Anticoagulant aptamers⁹
Aptamer Design with G-quadruplexes¹⁰
Nanotechnology¹¹
Biosensor design¹²

1. D. J. Patel, A. T. Phan and V. Kuryavyi, N. A. R., 2007, 35(22), 7429-7455. N. Jing et al., Cancer Res., 2004, 64(18), 6603-6609. G-quadruplexes as cancer targets & Breast tumors.

2. A. T. Phan, et al., P.N.A.S. USA, 2005, 102, 634-639. G-Tetrads as Potent HIV inhibitors

3. N. J. Ping & M. E. Hogan, J.B.C.,1998, 273-34992-34999. G tetrads as potent HIV inhibitors.

4. J. R. Wyatt etal., P.N.A.S. USA, 1994, 91, 1356-1360. potent anti HIV drug. G-Quartet aptamers

5. D. A. D Giusto & G. C. King, J.B.C., 2004, 279, 45, 46483-46489

6. C. G. Peng & M. J. Damha, N.A.R., 2007, 35(15), 4977-4988.

7. P. Schultze, N. V. Hud, F. W. Smith & J. Feigon; NAR, 1999, 27, 2, 3018-3028. Conformation of Dimeric quadruplexes with G rich sequences.

8. P. K. Dominick & M. B. Jarstfer; J. Am. Chem. Soc., 2004, 126, 18, 5050-5051 Folding topology of G-quadruplexes is controlled by conformational constrains.

9. K. Padmanabhan et al., J.B.C., 1993, 268, 17651-17654. Inhibition of Thrombin

10. C. F. Tang & R. H. Shafer, J. Am. Chem. Soc., 2006, 128: 5966-5973. Engineering Guanosine Quadruplexes.

11. P. Alberti & J. L. Mergny; P.N.A.S. USA, 2003, 100, 1569-1573. Nanotechnology

12. H. Ueyama , M. Takagi, S. Takenaka, J.A.C.S., 2002, 124, 14286-14287. Biosensor design

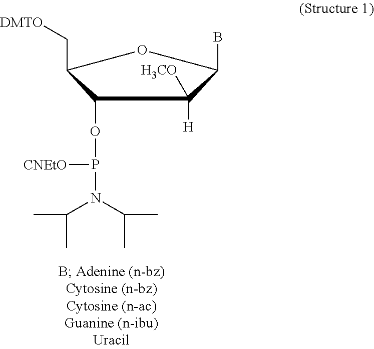

In formula 1A and 1B; wherein
Z is DMT, MMT, TMT

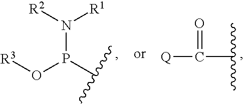

Q is a) a support comprised of a linking group and a spacer that can be cleaved to form a hydroxy group; or b) an aliphatic chain, aromatic group, substituted or unsubstituted aromatic, a substituted or unsubstituted phenoxy, or levulinyl;
R¹ is a substituted or unsubstituted (C₁-C₁₂)alkyl group, a substituted or unsubstituted (C₃-C₂₀)cycloalkyl group, or a substituted or unsubstituted (C₃-C₂₀)cycloalkyl(C₁-C₁₂)alkyl group, wherein the alkyl or cycloalkyl groups optionally include intervening heteroatoms independently selected from NH, NR⁷, O and S;
R² is a substituted or unsubstituted (C₁-C₁₂)alkyl group, a substituted or unsubstituted (C₃-C₂₀)cycloalkyl group, or a substituted or unsubstituted (C₃-C₂₀)cycloalkyl(C₁-C₁₂)alkyl group, wherein the alkyl or cycloalkyl groups optionally include intervening heteroatoms independently selected from NH, NR⁷, O and S;
or R¹ and R² taken together with the nitrogen atom to which they are bound form a 4-7 membered non-aromatic heterocyclyl, wherein the heterocyclyl formed may optionally include intervening heteroatoms independently selected from NH, NR⁷, O and S;
R³ is a phosphate protecting group;

Z is an acid labile protecting group;
B″ is hydrogen or an optionally substituted nucleobase optionally functionalized at each exocyclic amine with an amine protecting group, wherein the nucleobase is selected from:
N6, N6-dimethyl adenine, N6-benzoyladenine, N-1-methyladenine, 7-deazaadenine, 7-deaza-8-azaadenine, 3-deazaadenine, ethenoadenine, isoguanine, N1-methylguanine, 7-iodo-7-deazaguanine, 7-deaza-7-iodo adenine, 7-deaza-7-iodo-6-oxopurine, 5-iodo-5-methyl-7-deazaguanine, 7-deazaguanine substituted with —C≡C(CH₂)₁₋₈-pthlamide, 7-deaza-8-azaguanine, 8-methylguanine, 8-bromoguanine, 8-aminoguanine, hypoxanthine, 6-methoxypurine, 7-deaza-6-oxopurine, 6-oxopurine, 2-aminopurine, 2,6-diaminopurine, 8-bromopurine, 8-aminopurine, 8-alkylaminopurine, 8-alkylaminopurine, thymine, N-3 methyl thymine, 5-acroxymethylcytosine, 5-azacytosine, isocytosine, N-4(C₁-C₆)alkylcytosine, N-3(C₁-C₆)alkylcytidine, 5-propynylcytosine, 5-iodo-cytosine, 5-(C₁-C₆)alkylcytosine, 5-aryl(C₁-C₆)alkylcytosine, 5-trifluoromethylcytosine, 5-methylcytosine, ethenocytosine, cytosine and uracil substituted with —CH═CH—C(═O)NH(C₁-C₆)alkyl, cytosine and uracil substituted with —C≡C—CH₂-phthalimide, NH(C₁-C₆)alkyl, 4-thiouracil, 2-thiouracil, N³-thiobenzoylethyluracil, 5-propynyluracil, 5 Oacetoxymethyluracil, 5-fluorouracil, 5-chlorouracil, 5-bromouracil, 5-iodouracil, 4-thiouracil, N-3-(C₁-C₆) alkyluracil, 5-(3-aminoallyl)-uracil, 5-(C₁-C₆)alkyluracil, 5-aryl(C₁-C₆)alkyluracil, 5-trifluoro methyluracil, 4-triazolyl-5-methyluracil, 2-pyridone, 2-oxo-5-methylpyrimidine, 2-oxo-4-methylthio-5-methylpyrimidine, 2-thiocarbonyl-4-oxo-5-methylpyrimidine, and 4-oxo-5-methylpyrimidine;
wherein any substitutable nitrogen atom within the nucleobase or on the exocyclic amine is optionally substituted with fluorenylmethyloxycarbonyl; —C(═O)OPh; —C(═O)(C₁-C₁₆)alkyl; —C(═O)(C₂-C₁₆)alkenyl[edertz1]; —C(═O)(C₁-C₁₆)alkylene-C(═O)OH; —C(═O)(C₁-C₁₆)alkylene-C(═O)O(C₁-C₆)alkyl; [edertz2]=CR⁸N(C₁-C₆)alkyl)₂; —C(═O)—NR⁸—(CH₂)₁₋₁₆NR⁸C(═O)CF₃; —C(═O)—(CH₂)₁₋₁₆NR⁸C(═O)CF₃; —C(═O)—NR⁸(CH₂)₁₋₁₆NR⁸C(═O)-phthalimide; —C(═O)—(CH₂)₁₋₁₆-phthalimide; and

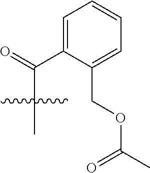

wherein any substitutable oxygen atom within the nucleobase is optionally substituted with —C(═O)N(C₁-C₆alkyl)₂—C(═O)N(phenyl)₂;

Further, other compounds based on 1A and 1B are:
1. The compound as described in the previous paragraphs, wherein the compound is represented by Formula IA or IB:
2. The compound of the previous paragraphs, wherein Z is an unsubstituted or substituted aryl group, an unsubstituted or substituted triarylmethyl group, an unsubstituted or substituted trityl group, an unsubstituted or substituted tetrahydropyranyl group, or an unsubstituted or substituted 9-phenylxanthyl.
3. The compound described above, wherein Z is di-p-anisylphenyl methyl, p-fluorophenyl-1-naphthylphenyl methyl, p-anisyl-1-naphthylphenyl methyl, di-o-anisyl-1-naphthyl methyl, di-o-anisylphenyl methyl, p-tolyldiphenylmethyl, di-p-anisylphenylmethyl, di-o-anisyl-1-naphthylmethyl, di-p-anisylphenyl methyl, di-o-anisyl phenyl methyl, di-p-anisylphenyl methyl, or p-tolyldiphenylmethyl.
4. The compound described above, wherein Z is represented by the following structural formula:

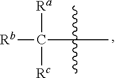

wherein [symbol] indicates attachment to the 3′ oxygen atom and R^{a}, R^{b}, and R^{c} are independently selected from the following structural formulas:

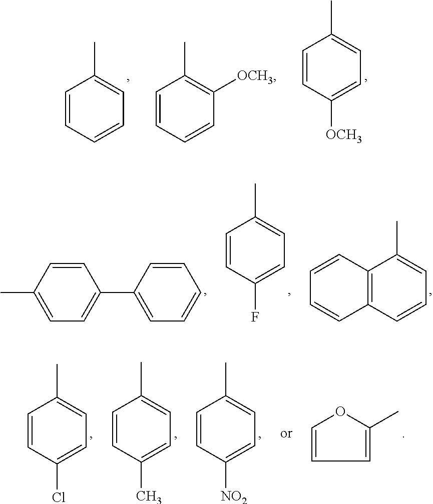

5. The compound, wherein Z is 4-methoxytrityl, 4,4′-dimethoxytrityl, or 4,4′,4″-trimethoxytrityl.
6. The compound, wherein the substitutable nitrogen atom within the nucleobase or on the exocyclic amine is optionally substituted with ═CHN(CH₃)₂; C(═O)CH(CH₃)₂; —C(═O)CH₃, ═C(CH₃)N(CH₃)₂; —C(═O)OPh; —C(═O)CH₂CH₂CH=CH₂; —C(═O)CH₂CH₂—C(═O)O(C₁-C₆)alkyl; —C(═O)—NR⁸—(CH₂)₁₋₁₆NR⁸C(═O)CF₃; —C(═O)—(CH₂)₁₋₁₆NR⁸C(═O)CF₃; —C(═O)—NR⁸(CH₂)₁₋₁₆NR⁸C(═O)-phthalimide; —C(═O)—(CH₂)₁₋₁₆-phthalimide

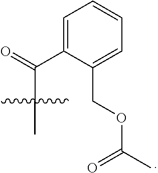

7. The compound, wherein R³ is —CH₂CH₂CN, —CH₂CH₂—Si(CH3)₂C₆H₅, —CH₂CH₂—S(O)₂—CH₂CH₃, —CH₂CH₂—C₆H₄—NO₂, —CH₂CH₂—NH—C(O)—C₆H₅, or —CH₂CH₂—O—C₆H₄—C(O)CH₃, and R⁴ is —O—Si(R¹¹)₃.
8. The compound, wherein the compound is represented by one of the following structural formulas:

The compound, wherein R³ is —CH₂CH₂CN.
wherein any substitutable nitrogen atom within the nucleobase or on the exocyclic amine in groups a), b), c), d) or e) is optionally substituted with (isobutyryl, phenoxyacetyl, tert-butylphenoxy acetyl, isopropyl phenoxyacetyl, acetyl, —C(O)OCH₃, di(C₁-C₆)alkylformamidine, p-chlorobenzoyl, o-chlorobenzoyl, o-nitrobenzoyl, p-nitrobenzoyl, fluorenylmethyloxycarbonyl, nitrophenylethyl, phthaloyl, Benzyl (Bn) group, p-Methoxybenzyl (PMB), 3,4-Dimethoxybenzyl (DMPM), p-methoxyphenyl (PMP) group and ═CR¹⁵N((C₁-C₆)alkyl)₂,
each R¹⁴ or R¹⁵ is a independently substituted or unsubstituted (C₁-C₆)alkyl group, a substituted or unsubstituted (C₂-C₆)alkenyl group, or a substituted or unsubstituted (C2-C₆)alkynyl group; and
each m is independently 0-12.

A protected nucleoside base is a nucleoside base in which reactive functional groups of the base are protected. Similarly, a protected heterocycle is a heterocycle in which reactive substituents of the heterocycle are protected. Typically, nucleoside bases or heterocycles have amine groups which can be protected with an amine protecting group, such as an amide or a carbonate. For example, the amine groups of adenine and cytosine are typically protected with benzoyl and alkyl ester, respectively, protecting groups, and the amine groups of guanine is typically protected with an isobutyryl group, an acetyl group or t-butylphenoxyacetyl group. However, other protection schemes may be used. For example, for fast deprotection, the amine groups of adenine and guanine are protected with phenoxyacetyl groups and the amine group of cytosine is protected with an isobutyryl group or an acetyl group. Conditions for removal of the nucleobase or heterocycle protecting group will depend on the protecting group used. When an amide protecting group is used, it can be removed by treating the oligonucleotide with a base solution, such as a concentrated ammonium hydroxide solution, N-methylamine solution or a solution of t-butylamine in ammonium hydroxide.

Nucleoside bases also include isocytidine (isoC) and isoguanosine (IsoG). IsoC and IsoG can used to exploit Watson Crick base pairing mechanism, which allow three hydrogen bonds between isoC and isoG, as shown below:

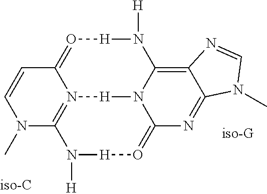

These bases can pair in both parallel and antiparallel duplex forms and as in DNA sequences they are recognized by DNA polymerases as well for chain extension, PCR etc. These molecules therefore have significant importance in as part of RNA sequences for diagnostics and therapeutic applications (See S. C. Jurczyk, et al., Helvetica Chimica Acta., 81, 793-811, 1998; and C. Roberts, et al., Tetrahedron Lett., 36, 21, 3601-3604, 1995, the entire teachings of which are incorporated herein by reference).

Nucleoside bases also include 7-deaza-ribonucleosides. These 7-deaza-ribonucleosides (including 7-deaza guanosine and adenosine and inosine) can be further modified at the 7 position by introducing various substituents. For example, modification can include attachment of halogen (such as fluoro, chloro, bromo, or iodo), alkynyl, trimethylsilylalkynyl, propynylaminotrifluoromethyl, or propynylamino phthalamido. (See Xiaohua Peng and Frank Seela, International Round Table on Nucleosides, Nucleotides and Nucleic Acids, IRT-XVII, Sep. 3-7, 2006, page 82, Bern, Switzerland, the entire teachings of which are incorporated herein by reference).

Specially, 7-deaza-2′-deoxy nucleosides can be incorporated within the RNA sequence in place of a dGuanosine base to result in a decrease clamping of oligodeoxy nucleotide and hence better resolution in sequence analysis. This modification does not decrease the tm values of sequences during hybridization to complementary sequences. This modification has many significant biological properties for diagnostic and therapeutic field of DNA and RNA (See N. Ramazaeva, et al., XIII International Round Table; Nucleosides, Nucleotides and Their Biological Application, Montpellier, France Sep. 6-10, 1998, poster 304; Ramazaeva, N., et al., Helv. Chim. Acta 1997, 80, 1809 and references cited therein; Sheela, F. et al., Helvetica Chimica Acta, 73: 1879, 1990, the entire teachings of which are incorporated herein). In RNA, the effect of G-C base pairing is much more pronounced because RNA molecules have a strong tendency to form secondary structures. Substitution of guanosine with 7-deaza-riboguanosine has great significance in RNA therapeutics and diagnostics. 7-substituted-7-deaza-ribonucleosides have significance due to possibility of various ligand and chromophore attachments at 7-position without disturbing G-C base pairing properties.

### Molecular Modeling of Ara-2′-O-Methyl nucleosides:

From molecular modeling experiments we observed that 2′-OMethyl-ara-guanosine residues in oligonucleotides forces the sugar in a rigid 2′-endo conformation (south/east). The Guanosine units have strong steric repulsion of bases and the β face of 2′-Ara-O-methyl group.

Nucleoside bases such as Ara-2′-Omethyl-adenosine, cytosine, uracil are seen to prefer the anti conformation.

Convenient Design of oligonucleotides with defined topology, such as parallel, antiparallel, cyclic array etc. with ara-2′-Omethyl modified bases.

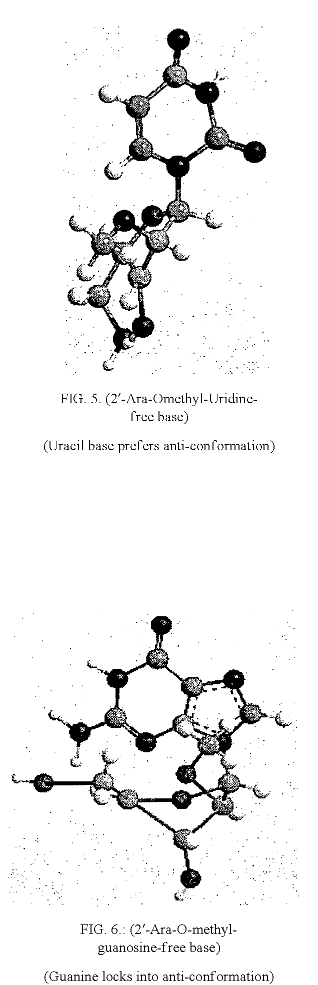

(2′)-O-Methyl-arabino nucleosides and triphosphaters-potential as new class of antimetabolite:

Besides the potential of (2′)-Omethyl-ara-bino nucleoside containing oligonucleosides,the nucleosides of the present research have potential applications in nucleoside bases therapeutics. Thus N-9-[β-D-Arabinofuranosyl]guanine (araG) is a Guanosine nucleoside analog that has shown higher efficiency in T-lymphoblasts compared to B-lymphoblasts. AraG is relatively resistant to degradation by purine nucleoside phosphorylase (PNP) and the selective cytotoxic effect on T-lymphoblasts is similar to that of deoxyguanosine in the absence of PNP activity. The molecular mechanism mediating this cell specific cytotoxicity of deoxyguanosine and its related analogs is poorly understood. However, a recent study suggests a role of mitochondria in this mechanism with intra-mitochondrial accumulation of dGTP and inhibition of DNA repair. The rate limiting step in araG phosphorylation to its triphosphate form is the initial phosphorylation to its monophosphate form, which is catalyzed by two different enzymes deoxyguanosine kinase (dGK) located in the mitochondrial matrix and deoxycytidine kinase (dCK) located in the cytosol of nucleus. Studies on purified dCK and dGK as well as analysis of araG phosphorylating activities in cell extracts suggest that dGK is the main phosphorylating enzyme of araG at lower concentrations whereas dCK seems to be more important at higher concentrations of araG. These results are consistent with the predominant incorporation of lower concentrations of araG into mtDNA. The dose toxicity in the clinical trials of Nelarabine, of araG, is neurotoxicity. Adverse effects also include myopathy, myelosuppression and the loss of pe sensitivity, similar to the symptoms of drugs mitrochondrial toxicity.

Nucleoside analogs, such as 1-[β-D-arabinofuranosylcytosine, 2-fluoro-2′-arabinofuranosyladenine and 2-chloro-deoxyadenosine, are commonly used in treatment of hematological malignancies. These compounds are transported across the cell membrane by nucleoside transporter proteins and phosphorylated intracellularly to their triphosphate derivatives by nucleoside and nucleotide kinases. The nucleoside analog triphosphates are subsequently incorporated into DNA and cause termination of DNA strand elongation or other DNA lesions. Replication of DNA occurs both in nucleus and in the mitochondrial matrix and there are accordingly two possible targets for nucleoside analogs.

### Nucleoside Antimetabolites:

It is expected that the nucleosides of our invention (formula 2A and 2B) can be used as therapeutic agents for treatment of many diseases such as cancer and virus infections, it is pertinent to discussed many present technology used in the area of nucleoside nbased antimetabolites. The outlined description presents overview of nucleoside antimetabolites and their potential in relation to cancer chemotherapy, antiviral agents is being described in the following section.

The outlined description presents overview of nucleoside antimetabolites and their potential in relation to cancer chemotherapy, antiviral agents is being described in the following section.

In combination chemotherapy utilizing two nucleoside based antimetabolites with one or combination of more than one nucleosides results in greater efficacy in treatment. Thus gemcitabine (dFdC) which is a new nucleoside antimetabolite of deoxycytidine that resembles cytarabine (Ara-C) in both its structure and metabolism and is also a nucleoside antimetabolite, were used in combination chemotherapy in leukemic cell growth. Similarly gemcitabine and other nucleoside antimetabolites in combination chemotherapy have been found to be very useful, normal and leukemic cell growth in vitro; E Lech-Maranda, A Korycka, and T Robak, Haematologica, 2000,Vol 85, Issue 6, 588-594 and it was shown that gemcitabine (dFdC) which is a new nucleoside antimetabolite of deoxycytidine that resembles cytarabine (Ara-C) in both its structure and metabolism and is also a nucleoside antimetabolite, were used in combination chemotherapy in leukemic cell growth.

Further references, applications, utilities, research notes and comments for this work include:

13. The Antiproliferative Activity of DMDC Is Modulated by Inhibition of Cytidine Deaminase; Cancer Research 58, 1165-1169, Mar. 15, 1998; Hiroyuki Eda, Masako Ura, Kaori F.-Ouchi, Yutaka Tanaka, Masanori Miwa and Hideo Ishitsuka

Summary: A new 2′-deoxycytidine (2′-dCyd) analogue, 2′-deoxy-2′-methylidenecytidine (DMDC), a nucleoside antimetabolite was found very promising as anticancer agent in multiple cancer cell lines. Study was carried to establish mode of actions and mechanism of action. Further combination chemotherapy of gemcitabine with another modified nucleoside tetrahydrouridine was evaluated and found to be encouraging.

14. Nucleoside analogues in the treatment of haematological malignancies; Expert Opin. Pharmacother., Jun. 1, 2001; 2(6): 929-43; S. A. Johnson.

Summary: This article reviews various nucleoside antimetabolites as cytotoxics. Many examples such as Cytrabine, Cladribine, fludarbine, gemcitabine, nelarabine, clofarabine and troxacitabine were chosen for detailed therapeutic properties/index. It is interesting to note that many of the anticancer agents are immunosuppressive in nature.

15. Synthesis of 1-(2-deoxy-2-isocyano-beta-D-arabinofuranosyl)cytosine and related nucleosides as potential antitumor agents; J. Med Chem, Dec. 24, 1993; 36(26): 4190-4; A. Matsuda, A. Dan, N. Minakawa, S. J. Tregear, S. Okazaki, Y. Sugimoto and T. Sasaki; Nucleosides and nucleotides. 123.

Summary: This publication details synthesis of a new chemical modification of several nucleoside related to nucleoside antimetabolite beta-D-arabinofuranosycytosine, nucleoside antimetabolite beta-D-artabinofuranosyuracil and nucleoside antimetabolite beta-D-artabinofuranosythymine. Only moderate antitumor activity was observed.

16. Nucleosides as Antimetabolites: Thioguanine, mercaptopurine: their analogs and nucleosides as antimetabolites. Curr Pharm Des, Jan. 1, 2003; 9(31): 2627-42; G. H. Elgemeie

Summary: This article presents an overview of well known purine based antimetabolites and various modifications of thiopurine based nucleoside antimetabolites. In light of many toxic side effects of the thiopurine based nucleoside antimetabolites other approaches and modifications are discussed as safe therapeutic agents.

17. Metabolism of pyrimidine analogues and their nucleosides; Pharmacol. Ther., Jan. 1, 1990; 48(2): 189-222; G. C. Daher, B. E. Harris, and R. B. Diasio

Summary: The article discusses the mode of action of nucleoside antimetabolites and specifically pyrimidine nucleoside antimetabolites and how they cause cytotoxic effect within the cellular environment. Four most common pyrimidine based nucleoside antimetabolites, viz., fluorouracil, fluorodeoxyuridine, cytosine arabinoside and azacytidine.

18. Transport of Nucleoside antimetabolites in Cancer Cells; Nucleoside anticancer drugs: the role of nucleoside transporters in resistance to cancer chemotherapy; Oncogene, Oct. 20, 2003; 22(47): 7524-36; V. L. Damaraju, S. Damaraju, J. D. Young, S. A. Baldwin, J. Mackey, M. B. Sawyer and C. E. Cass antimetabolite into cells and outlines various possible mechanisms

Summary: The article discusses mechanism of transport of nucleoside antimetabolite into cells and outlines various factors such as hENTs, hCNTs and their role in transport of cytotoxic chemotherapeutic nucleoside drugs. This understanding is very important towards the design of better nucleoside antimetabolites.

19. Potential Multifunctional Antitumor Nucleosides and Analogues; 1-(3-C-ethynyl-beta-D-ribo-pentofuranosyl)-cytosine, 1-(3-C-ethynyl-beta-D-ribo-pentofuranosyl)uracil, and their nucleobase analogues as new potential multifunctional antitumor nucleosides with a broad spectrum of activity; J. Med. Chem., Dec. 6, 1996; 39(25): 5005-11; H. Hattori, M. Tanaka, M. Fukushima, T. Sasaki, and A. Matsuda; Nucleosides and nucleotides. 158.

Summary: This article describes synthesis of new modifications (1-(3-C-ethynyl-beta-D-ribo-pentofuranosyl)uracil; EUrd) as a approach to develop multifunctional antitumor nucleoside antimetabolite. The authors introduced a “biochemically reactive” ethynyl group on uracil nucleoside resulting in modified uridine (beta-D-ribo-pentofuranosyl)uracil). However only moderate biological activity was observed.

20. Antitumor activity and pharmacokinetics of TAS-106, 1-(3-C-ethynyl-beta-D-ribo-pentofuranosyl)cytosine, Jpn. J. Cancer Res, Mar. 1, 2001; 92(3): 343-51, Y. Shimamoto, A. Fujioka, H. Kazuno, Y. Murakami, H. Ohshimo, T. Kato, A. Matsuda, T. Sasaki, and M. Fukushima.

Summary: This article similar to the preceding article describe synthesis of 3-C-ethynyl modification of cytosine nucleoside. This modification results in a modified nucleoside antimetabolite and possesses antitumor activity and strong cytotoxic effects useful for a cancer chemotherapy, and showed promise with lower less side effects.

21. Combinations of 5-fluorouracil and N-(2-Chloroethyl)-N-nitrosourea moieties separated by a three-carbon chain; The synthesis of antitumor activity in mice of molecular combinations of 5-fluorouracil and N-(2-Chloroethyl)-N-nitrosourea moieties separated by a three-carbon chain; J. Med. Chem., Mar. 29, 1996; 39(7): 1403-12; R. S. McElhinney, J. E. McCormick, M. C. Bibby, J. A. Double, M. Radacic, and P. Dumont; Nucleoside analogs. 14.

Summary: This article describes synthesis of modified nucleoside derived by combination of nucleoside antimetabolite, 5-fluoro uracil and attachment of N-(2-Chloroethyl)-N-nitrosourea moieties. Some antitumor activity was observed.

22. Modulation of the equilibrative nucleoside transporter by inhibitors of DNA synthesis; Br. J. Cancer, Oct. 1, 1995; 72(4): 939-42; J. Pressacco, J. S. Wiley, G. P. Jamieson, C. Erlichman, and D. W. Hedley

Summary: This study was carried out to measure and modulate the activity of sensitive nucleoside transporter (es), at the stage of de novo nucleoside synthesis pathway and thereby to regulate nucleoside antimetabolite. Inhibitors of DNA synthesis such as hydroxyurea and 5-fluorouracil (5-FU), which inhibit the de novo synthesis of DNA precursors, produced increases in the expression of es, while cytosine arabinoside (ara-C), another nucleoside antimetabolite produced no significant increase in es expression.

23. In Vitro Cell Dev Biol; Br. J. Cancer, Oct. 1, 1995; 72(4): 939-42. Nov. 1, 1991; 27A (11): 873-7; M. Moorghen, P. Ince, K. J. Finney, A. J. Watson, and A. L. Harris, Department of Pathology, University of Newcastle upon Tyne, United Kingdom

Summary: Nucleoside transport inhibitors modulate biological activity of nucleoside antimetabolites. The effect of nucleoside transport inhibitors such as nitrobenzylthioinosine (NBMPR) and dipyridamole which are responsible for binding with the enzymes responsible for transport of nucleosides was studied in this article.

24. Potentiation of the cytotoxicity of thymidylate synthase (TS) inhibitors by dipyridamole analogues with reduced alpha1-acid glycoprotein binding. Br. J. Cancer, Aug. 1, 1999; 80(11): 1738-46; N. J. Curtin, K. J. Bowman, R. N. Turner, B. Huang, P. J. Loughlin, A. H. Calvert, B. T. Golding, R. J. Griffin and D. R. Newell.

Summary: A new approach has been used by the authors to enhance the biological activity of the nucleoside antimetabolites by developing nucleoside transport inhibitors. A number of dipyridamole were shown to have potency of inhibition of uptake of nucleosides, there by inhibition of DNA synthesis.

25. Characterization of a multidrug resistant human erythroleukemia cell line (K562) exhibiting spontaneous resistance to 1-beta-D-arabinofuranosylcytosine; Leukemia, S. Grant, A. Turner, P. Nelms and S. Yanovich; May 1, 1995; 9(5): 808-14.

Summary: One of the key problems associated with anticancer drugs is multi drug resistance (MDR) during chemotherapy. In this article the authors studied the mechanism of MDR in the case of nucleoside analog antimetabolite 1-beta-D-arabinofuranosylcytosine (ara-C). Formation of the monophosphate of these nucleosides and the enzymes responsible for the phosphorylation seems to be the factor controlling resistance.

26. Clofarabine: Bioenvision/ILEX; Curr Opin Investig Drugs, A. Sternberg; Dec. 1, 2003; 4(12): 1479-87;

Summary: The article discusses a new modified nucleoside antimetabolite, Clofarabine which has shown significant promise for treatment of various forms of tumors and various forms of cancers.

27. Corticosteroid responsive fludarabine pulmonary toxicity, Am. J. Clin. Oncol., Aug. 1, 2002; 25(4): 340-1, G. S. Stoica, H. E. Greenberg and L. J. Rossoff. Division of Pulmonary and Critical Care Medicine, Long Island Jewish Medical Center, New Hyde Park, N.Y. 11042-1101, U.S.A

Summary: Not only modified nucleosides (with a free 5′-hydroxyl group) are nucleoside antimetabolites, but the corresponding 5′-mono phosphates of these nucleosides are also nucleoside antimetabolites and work with the same principle as the free 5′-hydroxyl nucleosides, i.e., getting incorporated during DNA synthesis and eventually stopping the DNA synthesis. Fludarabine, which has a fluoro group at 2-position was developed by introducing fluorine into a known nucleoside antimetabolite; Ara-A (9-beta-D-arabinofuranosyl adenine; vidarabine). The article reports clinical efficacy data on this nucleoside antimetabolite and the contribution to toxicity.

28. Cerebrospinal fluid pharmacokinetics and toxicology of intraventricular and intrathecal arabinosyl-5-azacytosine (fazarabine, NSC 281272) in the nonhuman primate. Invest New Drugs, May 1, 1993; 11(2-3): 135-40, R. L. Heideman, C. McCully, F. M. Balis and D. G. Poplack.

Summary: 5-aza-2′-deoxy cytidine and 5-aza-cytidine are highly potent anticancer drugs and presently used in cancer chemotherapy. Arabinosyl-5-azacytosine (AAC), a new nucleoside antimetabolite, is similar to 5-aza-2′-deoxy cytidine and 5-aza-cytidine in structure and has also shown strong anti tumor activity. The present article report clinical evaluation on primates.

29. Phase I trial and biochemical evaluation of tiazofurin administered on a weekly schedule, Sel. Cancer Ther., Mar. 1, 1990; 6(1): 51-61, T. J. Melink, G. Sarosy, A. R. Hanauske, J. L. Phillips, J. H. Bayne, M. R. Grever, H. N. Jayaram and D. D. Von Hoff.

Summary: The article reports pharmacological and biochemical study with another nucleoside antimetabolite, tiazofurin (2-B-D-Ribofuranosylthiazole-4-Carboxamide: NSC 286193). These are another class of nucleoside antimetabolites which act on the biosynthesis pathway of synthesis of purine nucleosides themselves. This leads to the inhibition of DNA synthesis and antitumor properties. However this compound was found to be associated with significant level of cellular toxicities.

30. Evaluation of purine and pyrimidine analogues in human tumor cells from patients with low-grade lymphoproliferative disorders using the FMCA, Eur. J. Haematol, May 1, 1999; 62(5): 293-9, Aleskog, R Larsson, M Hoglund, C Sundstrom, and J Kristensen.

Summary: This article reports clinical study data with few well established pyrimidine antimetabolites, fludarabine, cladribine (CdA), cytarabine (AraC) and gemcitabine. Cytotoxic studies carried out revealed effectiveness against non-Hodgkins lymphoma (NHL) is active against low-grade NHL and against acute leukemia. Gemcitabine and AraC were shown to be promising against low-grade NHL.

31. Altered susceptibility of differentiating HL-60 cells to apoptosis induced by antitumor drugs, Leukemia, Feb. 1, 1994; 8(2): 281-8, G Del Bino, X. Li, F. Traganos, and Z. Darzynkiewicz.

Summary: In this study it was shown that effectiveness of chemotherapeutic agents, nucleoside antimetabolite, including radiation is most likely reduced if a drug or chemical which has capability of cell cycle differentiation such as during S-phase or apoptosis and is administered first. In the converse, cell death or enhancement of apoptosis is expected if the cell cycle differentiating drug or chemical is administered in the reverse sequence.

32. Polarographic properties and potential carcinogenicity of some natural nucleosides and their synthetic analogues; Bioelectrochem Bioenerg, Feb. 1, 1999; 48(1): 129-34.; L. Novotny, A. Vachalkova, and A Piskala

Summary: A series of natural, synthetic nucleosides selected from a group of Nucleoside antimetabolites were studied for their potential carcinogenicity. It is interesting to note that various nucleoside antimetabolites do possess carcinogenicity.

33. This articles were taken from the book “Drug Resistance and Selectivity, Biochemical and Cellular Basis, Edited by Enrico Mihich, Roswell Park Memorial Cancer Institute, Buffalo, N.Y.; Academic Press, 1973, Pages 83-93, Chapter 3. CROSS-RESISTANCE AND COLLATERAL SENSITIVITY; Dorris J. Hutchinson and Franz A. Schmid”

Summary: The articles covering many purine and pyrimidine antimetabolites analogs are part of the chapter of this book Edited by E. Michich of Roswell Park Memorial Insititute, Buffalo, N.Y., and were written by many well known scientists involved in anticancer chemotherapy field in general. The cross resistance to anti cancer drugs and specifically nucleoside antimetabolite of modified purine and pyrimidine had been a serious issue and challenge recognized early on. Various mechanisms of this phenomenon were studied While nucleoside antimetabolites are very promising and effective in cancer chemotherapy, the issues addressed in the chapter have been serious and responsible for ineffectiveness and as well as associated significant cellular toxicity in general. The articles presents various approaches to address and overcome the shortcomings 34.Gemcitabine and Other Nucleoside Antimetabolites in Combination Chemotherapy; The interaction of gemcitabine and cytarabine on murine leukemias L1210 or P388 and on human normal and leukemic cell growth in vitro; Haematologica, Vol 85, Issue 6, 588-594;

E Lech-Maranda, A Korycka, and T Robak

BACKGROUND AND OBJECTIVE: Gemcitabine (dFdC) is a new nucleoside antimetabolite of deoxycytidine that resembles cytarabine (Ara-C) in both its structure and metabolism. Little is known about dFdC efficacy in hematologic malignancies, either as a single drug or in combination with other drugs. In this study we have tried to determine whether the cytotoxic effect of Ara-C can be increased by using it in combined therapy with dFdC. DESIGN AND METHODS: In the in vivo part of our study, mice bearing L1210 or P388 leukemia were treated with dFdC and Ara-C. The drugs were administered alone and in combination according to the following schedules: Ara-C and dFdC at the same time, dFdC before Ara-C, and Ara-C before dFdC. The efficacy of the therapy against leukemia (defined as the increase in lifespan, ILS) was assessed as the percentage of the median survival time (MST) of the treated group (T) in relationship to that of the control group (C): ILS=[(MST(C)/MST(T))−1]×100. In the in vitro part of our study, normal granulocyte-macrophage colony-forming unit (CFU-GM) cells as well as CFU-GM cells obtained from patients with chronic myeloid leukemia (CML) were incubated either with dFdC or Ara-C alone or with adequate concentrations of a combination of these drugs. RESULTS: The in vivo experiment revealed that in both leukemias tested, combined therapy with dFdC given before Ara-C and dFdC given at the same time with Ara-C were more effective than monotherapy with either dFdC or Ara-C. The other treatment schedule (Ara-C before dFdC) did not significantly prolong the survival time of the treated mice bearing L1210 or P388 leukemia as compared with the treatment with dFdC alone. The in vitro experiments showed that dFdC used together with Ara-C acted additively on normal as well as CML CFU-GM cells. Furthermore, the drugs used jointly inhibited the growth of colonies formed by CML CFU-GM cells to a significantly higher degree than normal CFU-GM and the differences were statistically significant in the case of the combination of highest concentrations. INTERPRETATION AND CONCLUSIONS: Gemcitabine increased the activity of Ara-C. As these agents incorporate into DNA blocking chain elongation, and moreover, dFdC influences the cytotoxicity of Ara-C, our results could be explained by the drugs acting at these levels. dFdC used jointly with Ara-C may have an important clinical implication in the treatment of CML and other hematologic malignancies in future.

Gemcitabine-containing regimens are among standard therapies for the treatment of advanced non-small cell lung,pancreatic, or bladder cancers. Gemcitabine is a nucleoside analogue and its cytotoxicity is correlated with incorporation into genomic DNA and concomitant inhibition of DNA synthesis. However, it is still unclear by which mechanism(s) gemcitabine incorporation leads to cell death.

Experimental Design: We used purified oligodeoxynucleotides to study the effects of gemcitabine incorporation on topoisomerase I (top1) activity and tested the role of top1 poisoning in gemcitabine-induced cytotoxicity in cancer cells.

Results: We found that top1-mediated DNA cleavage was enhanced when gemcitabine was incorporated immediately 3′ from a top1 cleavage site on the nonscissile strand. This position-specific enhancement was attributable to an increased DNA cleavage by top1 and was likely to have resulted from a combination of gemcitabine-induced conformational and electrostatic effects. Gemcitabine also enhanced camptothecin-induced cleavage complexes. We also detected top1 cleavage complexes in human leukemia CEM cells treated with gemcitabine and a 5-fold resistance of P388/CPT45 top1-deficient cells to gemcitabine, indicating that poisoning of top1 can contribute to the antitumor activity of gemcitabine.

Conclusions: The present results extend our recent finding that incorporation of 1-β-D-arabinofuranosylcytosine into DNA can induce top1 cleavage complexes [P. Pourquier et al. Proc. Natl. Acad. Sci. USA, 97: 1885-1890, 2000]. The enhancement of camptothecin-induced top1 cleavage complexes may, at least in part, contribute to the synergistic or additive effects of gemcitabine in combination with topotecan and irinotecan in human breast or lung cancer cells.

35. Inhibitory effects of the nucleoside analogue gemcitabine on prostatic carcinoma cells; Prostate, Mar. 1, 1996; 28(3): 172-81; M V Cronauer, H Klocker, H Talasz, F H Geisen, A Hobisch, C Radmayr, G Bock, Z Culig, M Schirmer, A Reissigl, G Bartsch, and G Konwalinka; Department of Urology, University of Innsbruck, Austria

Gemcitabine (2′,2′difluoro-2′deoxycytidine, dFdC) is a synthetic antimetabolite of the cellular pyrimidine nucleotide metabolism. In a first series of in vitro experiments, the drug showed a strong effect on the proliferation and colony formation of the human androgen-sensitive tumor cell line LNCaP and the androgen-insensitive cell lines PC-3 and DU-145. Maximal inhibition occurred at a dFdC concentration as low as 30 nM. In contrast to the cell lines which were derived from metastatic lesions of prostate cancer patients, no inhibitory effects were found in normal primary prostatic epithelial cells at concentrations up to 100 nM. The effect of gemcitabine was reversed by co-administration of 10-100 microM of its natural analogue deoxycytidine. In view of a future clinical application of this anti-tumor drug in advanced prostatic carcinoma, we have compared the effect of gemcitabine on prostatic tumor cells with that on bone marrow granulopoietic-macrophage progenitor cells, because neutropenia is a common side effect of gemcitabine treatment. The time course of action on the two kinds of cells was markedly different. Colony formation of tumor cells was inhibited by two thirds at a gemcitabine concentration of about 3.5 nM. The same effect on granulopoietic-macrophagic progenitor cells required a concentration of 9 nM. Co-administration of deoxycytidine to gemcitabine-treated tumor cell cultures completely antagonized the effect of gemcitabine whereas addition of deoxycytidine after 48 hr of gemcitabine treatment could not prevent gemcitabine action on the tumor cells. In contrast, more than half of the granulopoietic-macrophagic progenitor cells could still be rescued by deoxycytidine administration after 48 hr. These findings and the marked difference in the susceptibility of neoplastic and normal prostatic cells suggest that gemcitabine is a promising substance which should be further evaluated as to its efficacy in the treatment of advanced prostatic carcinoma.

36. Elaidic Acid—Ester of cytarabine (P-4055), a nucleoside antimetabolite; Antitumor Activity of P-4055 (Elaidic Acid-Cytarabine) Compared to Cytarabine in Metastatic and s.c. Human Tumor Xenograft Models: Biological activity in melanoma cells was found to be highly superior to that of cytarabine

Cancer Research 59, 2944-2949, Jun. 1, 1999; Knut Breistøl¹, Jan Balzarini, Marit Liland Sandvold, Finn Myhren, Marita Martinsen, Erik De Clercq and Øystein Fodstad

The antineoplastic efficacy of P-4055, a 5′-elaidic acid (C 18:1, unsaturated fatty acid) ester of cytarabine, a nucleoside antimetabolite frequently used in the treatment of hematological malignancies, was examined in several in vivo models for human cancer.

In initial dose-finding studies in nude mice, the efficacy of P-4055 was highest when using schedules with repeated dailydoses. In a Raji Burkitt's lymphoma leptomeningeal carcinomatosis model in nude rats, the control cytarabine- and saline-treated animals (five in each group) had a mean survival time of 13.2 days, whereas treatment with P-4055 resulted in three of five long-time survivors (>70 days). In a systemic Raji leukemia model in nude mice, 8 of 10 of the P-4055-treated animals survived (>80 days), compared with none of the cytarabine-treated animals (mean survival time, 34.2 days).

In s.c. xenograft models, the effects of maximum tolerated doses of P-4055 and cytarabine, given in four weekly cycles of daily bolus i.v. injections for 5 subsequent days, against seven tumors (three melanomas, one lung adenocarcinoma, one breast cancer, and two osteogenic sarcomas) were investigated. P-4055 induced partial or complete tumor regression of the lung carcinoma, as well as of all three malignant melanomas. In two of the melanomas the activity was highly superior to that of cytarabine, and both P-4055 and cytarabine were, in general, more effective than several clinically established drugs previously tested in the same tumor models. In in vitro studies, inhibitors of nucleoside carrier-dependent transport, nitrobenzylmercaptopurine riboside and dipyridamol, reduced strongly the cellular sensitivity to cytarabine, but not to P-4055, indicating that P-4055 uses an alternative/additional mechanism of internalization into the cell compared with cytarabine. The results explain, at least in part, the observed differences between the two compounds in in vivo efficacy, and together the data strongly support the evaluation of P-4055 in clinical studies.

37. Gemcitabine in the treatment of ovarian cancer; Int. J. Gynecol. Cancer, Jan. 1, 2001; 11 Suppl 1: 39-41; S W Hansen

Gemcitabine is a nucleoside antimetabolite with established activity against several solid tumors. The activity of the drug in patients with ovarian cancer has been reviewed both in patients who have received single drug treatment and in patients who have received combination chemotherapy. The response rates, with single agent gemcitabine, range from 13 to 24% both in previously treated and untreated patients. Doublets consisting of gemcitabine-cisplatin or gemcitabine-paclitaxel, in previously treated patients, induced response in 53% and 40% of the patients, respectively. In three studies, first-line treatment with the combination of cisplatin and gemcitabine induced remission in 53% to 71% of the patients. The triplet, including gemcitabine, paclitaxel, and cisplatin or carboplatin, has been examined in previously treated patients and a response rate of 100% was observed. In previously untreated patients the combination of gemcitabine, paclitaxel, and carboplatin has been preferred due to a more favorable toxicity profile. The activity of this combination, observed in 25 evaluable patients, was very high as all patients responded. Complete remission was observed in 60% of the patients and partial remission in 40%. Based on these promising data the triplet consisting of gemcitabine, paclitaxel, and carboplatin has been included in randomized trials both in the US and in Europe.

38. Gemcitabine in the treatment of ovarian cancer; Ann. Onc., Jan. 1, 1999; 10 Suppl 1: 51-3; S W Hansen, M K Tuxen, and C Sessa.

Gemcitabine is a new nucleoside antimetabolite with established activity against solid tumours. In previously treated patients the response rate with the drug alone was around 13%. Combination therapy with gemcitabine-cisplatin or gemcitabine-paclitaxel induced responses in 53 and 40% respectively. In previously untreated patients with poor prognostic features a 24% response rate was reported for the drug alone, but in combination with cisplatin remissions were found in 53%-71% of patients. Gemcitabine, paclitaxel, and carboplatin (or cisplatin) in combination appeared to be a feasible and active combination. In a pilot with eight previously treated patients all obtained a remission and in untreated patients a remission occurred in all evaluable patients either clinically or measured by a decrease of CA 125. Dose-limiting toxicity is mainly haematological.

39. Lack of in vivo crossresistance with gemcitabine against drug-resistant murine P388 leukemias; Cancer Chemother. Pharmacol., Jan. 1, 1996; 38(2): 178-80; W. R. Waud, K S Gilbert, G. B. Grindey, and J. F. Worzalla

Gemcitabine, a novel pyrimidine nucleoside antimetabolite, has shown clinical antitumor activity against several tumors (breast, small-cell and non-small-cell lung, bladder, pancreatic, and ovarian). We have developed a drug-resistance profile for gemcitabine using eight drug-resistant P388 leukemias in order to identify potentially useful guides for patient selection for further clinical trials of gemcitabine and possible non crossresistant drug combinations with gemcitabine. Multidrug-resistant P388 leukemias (leukemias resistant to doxorubicin or etoposide) exhibited no cross resistance to gemcitabine. Leukemias resistant to vincristine (not multidrug resistant), cyclophosphamide, melphalan, cisplatin, and methotrexate were also not cross resistant to gemcitabine. Only the leukemia resistant to 1-beta-D-arabinofuranosylcytosine was cross resistant to gemcitabine. The results suggest that (1) it may be important to exclude or to monitor with extra care patients who have previously been treated with 1-beta-D-arabinofuranosylcytosine and (2) the lack of cross resistance seen with gemcitabine may contribute to therapeutic synergism when gemcitabine is combined with other agents.

40. Phase II trial of gemcitabine in advanced sarcomas; Cancer, Jun. 15, 2002; 94(12): 3225-9; S. Okuno, J. Edmonson, M. Mahoney, J. C. Buckner, S. Frytak, and E. Galanis.

BACKGROUND: Care for patients with advanced sarcomas is mainly palliative. Gemcitabine, anucleoside antimetabolite, is an analog of deoxycytidine that has shown antitumor activity in several tumors. The aim of the current study was to determine the clinical activity of gemcitabine in patients with sarcomas. METHODS: The authors evaluated gemcitabine in patients with histologically confirmed sarcomas; one prior exposure to chemotherapy treatment was allowed. Prior radiation was allowed if given to non-indicator lesions. Treatment consisted of gemcitabine 1250 mg/m(2) intravenously over 30 minutes, every week×three, cycles repeated q28 days. RESULTS: Twenty nine of 30 patients were evaluable; one patient refused to initiate study treatment. The mean age was 50 years (range, 22-81 years); 59% were male, and 35% had an Eastern Cooperative Oncology Group performance status of 0 (vs. 1 or 2). Patients were histologically classified as leiomyosarcoma (seven gastrointestinal, four retroperitoneal, two inferior vena caval, three of the extremity, and two uterine), synovial (two patients), malignant fibrous histiocytoma (two patients), fibrosarcoma (one patient), osteosarcoma (two patients), liposarcoma (one patient), hemangiosarcoma (one patient), or giant cell (one patient). Patients received an average of two cycles (range, one to eight). Eighty three percent of patients discontinued treatment due to progression and 14% due to toxicity/refusal. Hematologic toxicities > or = Grade 3 were seen in 32% of patients and consisted of leukopenia and thrombocytopenia. Anorexia (Grade ½ in 6 patients, Grade 3 in 1 patient), nausea (Grade ½ in 7 patients, Grade 3 in 1 patient), and lethargy (Grade ½ in 19 patients) were the most frequently observed nonhematologic toxicities. One patient experienced Grade 3 edema and muscle infarction. A different patient experienced unexplained Grade 3 chest pain. One partial response was observed in a uterine leiomyosarcoma patient lasting at least three months. Overall response rate was 3% (95% confidence interval [CI]: 0-15). Median time-to progression was 2.1 months (95% CI: 1.8-3.0). CONCLUSIONS: The current gemcitabine regimen demonstrated acceptable levels of toxicity, but it failed to produce the number of responses needed to justify expansion of the current study. This regimen is not recommended for advanced sarcomas.

41: Gemcitabine: a pharmacologic and clinical overview; Cancer Nurs., Apr. 1, 1999; 22(2): 176-83; M. Barton-Burke.

There have been exciting new developments in anticancer therapy over the past few years. One such therapy uses gemcitabine (GemzarR), an antimetabolite approved in 1996 by the Food and Drug Administration (FDA) for first-line treatment of locally advanced (nonresectable stage II or stage III) or metastatic (stage IV) adenocarcinoma of the pancreas.

This novel nucleoside analog resembles the naturally occurring pyrimidine nucleoside deoxycytidine, but it has a unique mechanism of action. Clinical studies with gemcitabine have demonstrated anticancer activity in pancreatic cancer; non-small-cell lung cancer; breast, bladder, and ovarian cancers; and small-cell lung cancer. Clinical trials in patients with cancer of the pancreas used a novel study end point called clinical benefits response (CBR) to measure gemcitabine's effect on disease-related symptoms. The CBR is a composite assessment of performance status, pain, and weight gain. Studies show that gemcitabine has a relatively mild safety profile, with myelosuppression as the major dose-limiting toxicity. The aim of this review is to provide the oncology nurse with an overview of gemcitabine's pharmacology, innovative clinical trial end points, and clinical performance, as well as the nursing care required for the patient receiving this drug.

42. Gemcitabine and carboplatin for patients with advanced non-small cell lung cancer. Semin Oncol, Jun. 1, 2001; 28(3 Suppl 10): 4-9; Domine, V. Casado, L. G. Estevez, A. Leon, J. I. Martin, M. Castillo, G. Rubio, and F. Lobo.

The survival of patients with advanced non-small cell lung cancer remains poor. Cisplatin-based chemotherapy produces a modest benefit in survival compared with that observed with best supportive care. Gemcitabine (Gemzar; Eli Lilly and Company, Indianapolis, Ind.), a novel nucleoside antimetabolite, is active and well tolerated. The combination of gemcitabine/cisplatin has shown a significant improvement in response rate and survival over cisplatin alone. Phase III trials comparing gemcitabine/cisplatin with older combinations such as cisplatin/etoposide or mitomycin/ifosfamide/cisplatin have shown a higher activity for gemcitabine/cisplatin; however, the best way to combine these drugs remains unclear. In addition, the 3-week schedule has obtained a higher dose intensity with less toxicity and similar efficacy as the 4-week schedule. The role of carboplatin in combination with new drugs is still under evaluation. Gemcitabine/carboplatin seems to be a good alternative, with the advantage of ambulatory administration and lower nonhematologic toxicity. The 4-week schedule has produced frequent grade 3/4 neutropenia and thrombocytopenia in some studies. The 3-week schedule, using gemcitabine on days 1 and 8 and carboplatin on day 1, is a convenient and well-tolerated regimen. The toxicity profile is acceptable without serious symptoms. This schedule could be considered a good option as a standard regimen.

43. Preliminary evaluation of influence of gemcitabine (Gemzar) on proliferation and neuroendocrine activity of human TT cell line: immunocytochemical investigations. Folia Histochem. Cytobiol., Jan. 1, 2001; 39(2): 187-8; J. Dadan, S. Wolczynski, B. Sawicki, L. Chyczewski, A. Azzadin, J. Dzieciol, and Z. Puchalski.

The choice treatment of medullary thyroid carcinoma (MTC) is total thyroidectomy. It is difficult to evaluate effectiveness of chemotherapy due to the rare incidence of MTC. Gemcitabine is a new drug of antimetabolite nucleoside group used in treatment of cancers since 1996. The aim of this study was to evaluate the influence of gemcitabine on proliferation and neuroendocrine activity of human TT cell line derived from MTC. The cells were exposed to gemcitabine in the concentration of 10, 25 and 50 microg/ml for 24 hours. Immunocytochemical examinations were carried out by the method of avidin-biotin peroxidase complex (ABC) according to Hsu et al. to detect calcitonin, chromogranin A, synaptophysin and neuron-specific enolase in TT cells. A concentration-dependent inhibitory influence of gemcitabine on proliferative activity of TT cells was observed. It was also shown that the immunostaining was reduced, especially in case of neuron-specific enolase. Only the reaction detecting calcitonin was enhanced in persisting.

A concentration-dependent inhibitory influence of gemcitabine on proliferative activity of TT cells was observed. It was also shown that the immunostaining was reduced,has action mechanisms similar to those of DMDC, is only slightly active in tumors with higher levels of the enzyme. In the present study, we investigated the roles of Cyd deaminase in the antitumor activity of the two 2′-dCyd antimetabolites in 13 human cancer cell lines.

Tetrahydrouridine, an inhibitor of Cyd deaminase, reduced the antiproliferative activity of DMDC (P=0.0015). Furthermore, tumor cells transfected with the gene of human Cyd deaminase become more susceptible to DMDC both in vitro and in vivo. These results indicate that Cyd deaminase is indeed essential for the activity of DMDC. In contrast, the antiproliferative activity of gemcitabine was increased to some extent by tetrahydrouridine (P=0.0277), particularly in tumor cell lines with higher levels of Cyd deaminase. This suggests that higher levels of Cyd deaminase may inactivate gemcitabine.

Among nucleosides and deoxynucleosides tested, only dCyd, a natural substrate of both Cyd deaminase and dCyd kinase, suppressed the antiproliferative activity of DMDC by up to 150-fold. Because the VₘₐₓKₘ of DMDC for dCyd kinase was 8-fold lower than that for dCyd, the activation of DMDC to DMDC monophosphate (DMDCMP) by dCyd kinase might be competitively inhibited by dCyd. In addition, the dCyd concentrations in human cancer xenografts were inversely correlated with levels of Cyd deaminase activity. It is therefore suggested that higher levels of Cyd deaminase reduce the intrinsic cellular concentrations of dCyd in tumors, resulting in efficient activation of DMDC to DMDCMP by dCyd kinase. These results indicate that the efficacy of DMDC may be predicted by measuring the activity of Cyd deaminase in tumor tissues before treatment starts and that DMDC may be exploited in a new treatment modality: tumor enzyme-driven cancer chemotherapy.

44. The Antiproliferative Activity of DMDC Is Modulated by Inhibition of Cytidine Deaminase; Cancer Research 58, 1165-1169, Mar. 15, 1998; Hiroyuki Eda, Masako Ura, Kaori F.-Ouchi, Yutaka Tanaka, Masanori Miwa and Hideo Ishitsuka

We showed that the efficacy of the new 2′-deoxycytidine (2′-dCyd) analogue antimetabolite 2′-deoxy-2′-methylidenecytidine (DMDC) correlates well with tumor levels of cytidine (Cyd) deaminase in human cancer xenograft models. DMDC was highly effective in tumors with higher levels of Cyd deaminase, whereas lower levels yielded only slight activity. In contrast, gemcitabine (2′,2′-difluorodeoxycytidine), which has action mechanisms similar to those of DMDC, is only slightly active in tumors with higher levels of the enzyme. In the present study, we investigated the roles of Cyd deaminase in the antitumor activity of the two 2′-dCyd antimetabolites in 13 human cancer cell lines. Tetrahydrouridine, an inhibitor of Cyd deaminase, reduced the antiproliferative activity of DMDC (P=0.0015). Furthermore, tumor cells transfected with the gene of human Cyd deaminase become more susceptible to DMDC both in vitro and in vivo. These results indicate that Cyd deaminase is indeed essential for the activity of DMDC. In contrast, the antiproliferative activity of gemcitabine was increased to some extent by tetrahydrouridine (P=0.0277), particularly in tumor cell lines with higher levels of Cyd deaminase. This suggests that higher levels of Cyd deaminase may inactivate gemcitabine.

Among nucleosides and deoxynucleosides tested, only dCyd, a natural substrate of both Cyd deaminase and dCyd kinase, suppressed the antiproliferative activity of DMDC by up to 150-fold. Because the VₘₐₓKₘ of DMDC for dCyd kinase was 8-fold lower than that for dCyd, the activation of DMDC to DMDC monophosphate (DMDCMP) by dCyd kinase might be competitively inhibited by dCyd. In addition, the dCyd concentrations in human cancer xenografts were inversely correlated with levels of Cyd deaminase activity. It is therefore suggested that higher levels of Cyd deaminase reduce the intrinsic cellular concentrations of dCyd in tumors, resulting in efficient activation of DMDC to DMDCMP by dCyd kinase. These results indicate that the efficacy of DMDC may be predicted by measuring the activity of Cyd deaminase in tumor tissues before treatment starts and that DMDC may be exploited in a new treatment modality: tumor enzyme-driven cancer chemotherapy.

45. Nucleoside analogues in the treatment of haematological malignancies; Expert Opin. Pharmacother., Jun. 1, 2001; 2(6): 929-43; S. A. Johnson.

The nucleoside analogues are a group of antimetabolite cytotoxics which generally have to be metabolized to the equivalent nucleotide before incorporation into DNA. Cytarabine is a well established component of the treatment of acute leukaemias and has its principal action on dividing cells. New formulations include a liposome encapsulated product for intrathecal use and oral cytarabine ocfosfate which may be suitable for long-term outpatient use. Pentostatin acts by causing accumulation of deoxynucleotides and, although active against hairy cell leukaemia, is associated with a poor tolerance profile. Cladribine and fludarabine have substantial activity in the treatment of chronic lymphocytic leukaemia (CLL) and low-grade non-Hodgkin's lymphoma (NHL). Fludarabine is the more thoroughly investigated of the two and is currently being developed in combination therapies for CLL and NHL and also in a combination with cytarabine for acute myeloid leukaemia. Fludarabine's immunosuppressive activity is being exploited in the conditioning of patients for non-myeloablative stem cell transplantation. Gemcitabine is an established agent in the treatment of a number of solid tumours but also has activity in haematological malignancies which might be exploited by the use of extended infusion schedules. Newer agents including nelarabine, clofarabine and troxacitabine are undergoing clinical evaluation and show promising activity.

46. Synthesis of 1-(2-deoxy-2-isocyano-beta-D-arabinofuranosyl)cytosine and related nucleosides as potential antitumor agents; J Med Chem, Dec. 24, 1993; 36(26): 4190-4; A. Matsuda, A. Dan, N. Minakawa, S. J. Tregear, S. Okazaki, Y. Sugimoto and T. Sasaki; Nucleosides and nucleotides. 123.

2′-Deoxy-2′-isocyano-1-beta-D-arabinofuranosylcytosine (8, NCDAC) has been synthesized as a potential antitumor antimetabolite from a corresponding 2′-azido-2′-deoxy-1-beta-D-arabinofuranosyluracil derivative 2a. Uracil and thymine analogues 6a and 6b of 8 were also prepared. Attempts to synthesize 2′-deoxy-2′-isocyanocytidine (14b) failed due to the insertion of the 2′-alpha isocyano group into the 3′-OH group, affording the 2′,3′-oxazoline derivative 15b. Stability of the isocyano derivative 6a and 2′,3′-oxazoline derivative 15a under basic and acidic conditions were examined. The isocyano group in 6a was stable in basic conditions but unstable even in weakly acidic conditions to furnish the corresponding 2′-beta formamide derivative 17. Compound 15a was easily hydrolyzed the corresponding 2′-alpha formamide derivative 16 on treatment with H₂O at room temperature. The cytotoxicity of 8, 6a, and 6b was examined in mouse and human tumor cells in vitro and compared with that of ara-C. Of these nucleosides, 8 was moderately cytotoxic to these cell lines. In vivo antitumor activity of 8 against Lewis lung carcinoma cells was also investigated and 8 showed only moderate tumor volume inhibition.

47. Nucleosides as Antimetabolites: Thioguanine, mercaptopurine: their analogs and nucleosides as antimetabolites. Curr Pharm Des, Jan. 1, 2003; 9(31): 2627-42; G. H. Elgemeie

Mercaptopurine (6MP) and 6-thioguanine (6TG) are analogs of the natural purines: hypoxanthine and guanine. Both mercaptopurine and thioguanine are substrates for hypoxanthine-guanine phosphoribosyltransferase and are converted into the ribonucleotides 6-thioguanosine monophosphate (6-thioGMP) and 6-thioinosine monophosphate (T-IMP) respectively. The accumulation of these monophosphates inhibits several vital metabolic reactions. Today, these thiopurine bases remain valuable agents for the induction and maintenance of remissions in patients with myelocytic and acute lymphocytic leukemia. Despite their proved clinical importance, 6MP and 6TG have certain therapeutic disadvantages, which have continued to stimulate the search for purine derivatives enhancing therapeutic efficacy. Considerable efforts have been made to prepare other novel mercaptopurine and thioguanine analogs and their nucleosides to improve the antitumor efficacy. The effectiveness of these thiopurines against certain tumor cell lines suggested that some of these mercaptopurine analogs and their nucleosides would be worthy of consideration in order to determine whether they exert a more selective effect against neoplastic cells than against normal cells or they might be useful in patients whose disease has become resistant to 6MP or 6TG. This review will focus on mercaptopurine analogs and their nucleosides as antimetabolite reagents.

48. Metabolism of pyrimidine analogues and their nucleosides; Pharmacol. Ther., Jan. 1, 1990; 48(2): 189-222; G. C. Daher, B. E. Harris, and R. B. Diasio

The pyrimidine antimetabolite drugs consist of base and nucleoside analogues of the naturally occurring pyrimidines uracil, thymine and cytosine. As is typical of antimetabolites, these drugs have a strong structural similarity to endogenous nucleic acid precursors. The structural differences are usually substitutions at one of the carbons in the pyrimidine ring itself or substitutions at on of the hydrogens attached to the ring of the pyrimidine or sugar (ribose or deoxyribose). Despite the differences noted above, these analogues, can still be taken up into cells and then metabolized via anabolic or catabolic pathways used by endogenous pyrimidines. Cytotoxicity results when the antimetabolite either is incorporated in place of the naturally occurring pyrimidine metabolite into a key molecule (such as RNA or DNA) or competes with the naturally occurring pyrimidine metabolite for a critical enzyme. There are four pyrimidine antimetabolites that are currently used extensively in clinical oncology. These include the fluoropyrimidines fluorouracil and fluorodeoxyuridine, and the cytosine analogues, cytosine arabinoside and azacytidine.

49. Transport of Nucleoside antimetabolites in Cancer Cells; Nucleoside anticancer drugs: the role of nucleoside transporters in resistance to cancer chemotherapy; Oncogene, Oct. 20, 2003; 22(47): 7524-36; V. L. Damaraju, S. Damaraju, J. D. Young, S. A. Baldwin, J. Mackey, M. B. Sawyer and C. E. Cass antimetabolite into cells and outlines various possible mechanisms

The clinical efficacy of anticancer nucleoside drugs depends on a complex interplay of transporters mediating entry of nucleoside drugs into cells, efflux mechanisms that remove drugs from intracellular compartments and cellular metabolism to active metabolites. Nucleoside transporters (NTs) are important determinants for salvage of preformed nucleosides and mediated uptake of antimetabolite nucleoside drugs into target cells. The focus of this review is the two families of human nucleoside transporters (hENTs, hCNTs) and their role in transport of cytotoxic chemotherapeutic nucleoside drugs. Resistance to anticancer nucleoside drugs is a major clinical problem in which NTs have been implicated. Single nucleotide polymorphisms (SNPs) in drug transporters may contribute to interindividual variation in response to nucleoside drugs. In this review, we give an overview of the functional and molecular characteristics of human NTs and their potential role in resistance to nucleoside drugs and discuss the potential use of genetic polymorphism analyses for NTs to address drug resistance.

50. Potential Multifunctional Antitumor Nucleosides and Analogues; 1-(3-C-ethynyl-beta-D-ribo-pentofuranosyl)-cytosine, 1-(3-C-ethynyl-beta-D-ribo-pentofuranosyl)uracil, and their nucleobase analogues as new potential multifunctional antitumor nucleosides with a broad spectrum of activity; J. Med. Chem., Dec. 6, 1996; 39(25): 5005-11; H. Hattori, M. Tanaka, M. Fukushima, T. Sasaki, and A. Matsuda; Nucleosides and nucleotides. 158.

We previously designed 1-(3-C-ethynyl-beta-D-ribo-pentofuranosyl)uracil (EUrd) as a potential multifunctional antitumor nucleoside antimetabolite. It showed a potent and broad spectrum of antitumor activity against various human tumor cells in vitro and in vivo. To determine the structure-activity relationship, various nucleobase analogues of EUrd, such as 5-fluorouracil, thymine, cytosine, 5-fluorocytosine, adenine, and guanine derivatives, were synthesized by condensation of 1-O-acetyl-2,3,5-tri-O-benzoyl-3-C-ethynyl-alpha,beta-D-ribo-pentofuranose (6) and the corresponding pertrimethylsilylated nucleobases in the presence of SnCl4 or TMSOTf as a Lewis acid in CH3CN followed by debenzoylation. The in vitro tumor cell growth inhibitory activity of these 3′-C-ethynyl nucleosides against mouse leukemia L1210 and human nasopharyngeal KB cells showed that 1-(3-C-ethynyl-beta-D-ribo-pentofuranosyl)cytosine (ECyd) and EUrd were the most potent inhibitors in the series, with IC50 values for L1210 cells of 0.016 and 0.13 microM and for KB cells of 0.028 and 0.029 microM, respectively. 5-Fluorocytosine, 5-fluorouracil, and adenine nucleosides showed much lower activity, with 1050 values of 0.4-2.5 microM, while thymine and guanine nucleosides did not exhibit any activity up to 300 microM. We next evaluated the tumor cell growth inhibitory activity of ECyd and EUrd against 36 human tumor cell lines in vitro and found that they were highly effective against these cell lines with 1050 values in the nanomolar to micromolar range. These nucleosides have a similar inhibitory spectrum. The in vivo antitumor activities of ECyd and EUrd were compared to that of 5-fluorouracil against 11 human tumor xenografts including three stomach, three colon, two pancreas, one renal, one breast, and one bile duct cancers. ECyd and EUrd showed a potent tumor inhibition ratio (73-92% inhibition relative to the control) in 9 of 11 and 8 of 11 human tumors, respectively, when administered intravenously for 10 consecutive days at doses of 0.25 and 2.0 mg/kg, respectively, while 5-fluorouracil showed potent inhibitory activity against only one tumor. Such excellent antitumor activity suggests that ECyd and EUrd are worth evaluating further for use in the treatment of human cancers.

51. Antitumor activity and pharmacokinetics of TAS-106, 1-(3-C-ethynyl-beta-D-ribo-pentofuranosyl)cytosine, Jpn. J. Cancer Res, Mar. 1, 2001; 92(3): 343-51, Y. Shimamoto, A. Fujioka, H. Kazuno, Y. Murakami, H. Ohshimo, T. Kato, A. Matsuda, T. Sasaki, and M. Fukushima.

We examined the effects of dosage schedule on antitumor activity in vitro and in vivo to determine the optimal administration schedule for a new nucleoside antimetabolite 1-(3-C-ethynyl-beta-D-ribo-pentofuranosyl)cytosine (ECyd, TAS-106). The cytotoxicity of TAS-106 in vitro against human tumors was evaluated at three drug exposure periods. TAS-106 exhibited fairly potent cytotoxicity even with 4 h exposure, and nearly equivalent and sufficiently potent cytotoxicity with 24 and 72 h exposures. These results suggest that long-term exposure to TAS-106 will not be required to achieve maximal cytotoxicity. The antitumor activity of TAS-106 in vivo was compared in nude rat models bearing human tumors on three administration schedules, once weekly, 3 times weekly, and 5 times weekly for 2 or 4 consecutive weeks. TAS-106 showed strong antitumor activity without serious toxicity on all three schedules, but the antitumor activity showed no obvious schedule-dependency in these models. When tumor-bearing nude rats were given a single i.v. dose of [(3)H]TAS-106, tumor tissue radioactivity tended to remain high for longer periods of time as compared to the radioactivity in various normal tissues. Furthermore, when the metabolism of TAS-106 in the tumor was examined, it was found that TAS-106 nucleotides (including the active metabolite, the triphosphate of TAS-106) were retained at high concentrations for prolonged periods. These pharmacodynamic features of TAS-106 may explain the strong antitumor activity without serious toxicity, observed on intermittent administration schedules, in nude rat models with human tumors. We therefore consider TAS-106 to be a promising compound which merits further investigation in patients with solid tumors.

52. Combinations of 5-fluorouracil and N-(2-Chloroethyl)-N-nitrosourea moieties separated by a three-carbon chain; The synthesis of antitumor activity in mice of molecular combinations of 5-fluorouracil and N-(2-Chloroethyl)-N-nitrosourea moieties separated by a three-carbon chain; J. Med. Chem., Mar. 29, 1996; 39(7): 1403-12; R. S. McElhinney, J. E. McCormick, M. C. Bibby, J. A. Double, M. Radacic, and P. Dumont; Nucleoside analogs. 14.

5-fluorouracil (5-FU) seco-nucleosides having as the “sugar” moiety a two-carbon (C2) side chain carrying a N-(2-chloroethyl)-N-nitrosourea group were designed as molecular combinations of antimetabolite and alkylating agent, but hydrolytic release of free 5-FU was not fast enough for significant contribution to the high activity they showed against colon and breast tumors in mice. In the present study of the synthesis of the more reactive C3 seco-nucleosides, it emerged that, of various groups attached to the aldehydic center in the precursor phthalimides, only the alkoxy/uracil-1-yl type was conveniently obtained by the standard method. The methylthio/uracil-1-yl analog required relatively large amounts of reagent methanethiol, and exploration of alternatives involving alpha-chlorination of alkyl methyl sulfide or Pummerer rearrangement of its S-oxide, or successive hydrolysis and methylation of isothiouronium bromide, gave disappointing yields. For successful preparation of the alkoxy/uracil-3-yl compounds, the route used for C2 homologs required considerable experimental modification. In addition to these O,N- and S,N-acetals, some N,N-acetals bearing two 5-FU residues were prepared. The new drugs have been tested against a panel of experimental tumors in mice. Although it is evident from a parallel study that even these C3 seco-nucleosides release free 5-FU too slowly in vivo, several of them have shown impressive anticancer activity. Reviewing their performance in comparison with earlier molecular combinations, a short list of seven [B.4152 (6), B.4015 (5), B.4030 (10), B.3999 (4), B.3995 (2), B.4083 (3), and B.3996 (the N 3-substituted analog of 1)] should be investigated further. This is particularly appropriate in light of the present understanding of the mode of action of chloroethylating agents. Following a prolonged period of clinical impatience with nitrosoureas because of limited selectivity action, a new era is confidently anticipated as these powerful drugs are increasingly studied in combination with O6-benzylguanine and other more efficient inhibitors of repair enzymes like O6-alkylguanine-DNA-alkyltransferase now being developed.

53. Modulation of the equilibrative nucleoside transporter by inhibitors of DNA synthesis; Br. J. Cancer, Oct. 1, 1995; 72(4): 939-42; J. Pressacco, J. S. Wiley, G. P. Jamieson, C. Erlichman, and D. W. Hedley

Expression of the equilibrative, S-(p-nitrobenzyl)-6-thioinosine (NBMPR)-sensitive nucleoside transporter (es), a component of the nucleoside salvage pathway, was measured during unperturbed growth and following exposure to various antimetabolites at growth-inhibitory concentrations. The probe 5-(SAENTA-x8)-fluorescein is a highly modified form of adenosine incorporating a fluorescein molecule. It binds. with high affinity and specificity to the (es) nucleoside transporter at a 1:1 stoichiometry, allowing reliable estimates of es expression by flow cytometry. Using a dual labelling technique which combined the vital DNA dye Hoechst-33342 and 5-(SAENTA-x8)-fluorescein, we found that surface expression of es approximately doubled between G1 and G2+M phases of the cell cycle. To address the question of whether es expression could be modulated in cells exposed to drugs which inhibit de novo synthesis of nucleotides, cells were exposed to antimetabolite drugs having different modes of action. Hydroxyurea and 5-fluorouracil (5-FU), which inhibit the de novo synthesis of DNA precursors, produced increases in the expression of es. In contrast, cytosine arabinoside (ara-C) and aphidicolin, which directly inhibit DNA synthesis, produced no significant increase in es expression. Thymidine (TdR), which is an allosteric inhibitor of ribonucleotide reductase that depletes dATP, dCTP and dGTP pools while repleting the dTTP pool, had no significant effect on es expression. These data suggest that surface expression of the esnucleoside transporter is regulated by a mechanism which is sensitive to the supply of deoxynucleotides. Because 5-FU (which specifically depletes dTTP pools) causes a large increase in expression whereas TdR (which depletes all precursors except dTTP) does not, this mechanism might be particularly sensitive to dTTP pools.

54. In Vitro Cell Dev Biol; Br. J. Cancer, Oct. 1, 1995; 72(4): 939-42. Nov. 1, 1991; 27A (11): 873-7; M. Moorghen, P. Ince, K. J. Finney, A. J. Watson, and A. L. Harris, Department of Pathology, University of Newcastle upon Tyne, United Kingdom

The in-vitro effects of hydroxyurea, nucleoside antimetabolites 5-FU and 5-FUdR have been extensively studied in experimental systems employing cell-line techniques. In this study we investigated the effects of these drugs on the levels of incorporation of labeled nucleosides into DNA in explants of intact rat colonic mucosa maintained in organ culture. The effects of the nucleoside transport inhibitors nitrobenzylthioinosine (NBMPR) and dipyridamole—which are modulators of antimetabolite cytotoxicity, on the incorporation of tritiated thymidine ([3H]TdR) into DNA were also studied. The incorporation of tritiated TdR into DNA was reduced by hydroxyurea but was not altered by either 5-FU or 5-FUdR. The levels of tritiated deoxyuridine were reduced by 5-FU and 5-FUdR in separate experiments; this is in keeping with thymidylate synthase inhibition. NBMPR and dipyridamole also reduced 3H-TdR incorporation into DNA. These results can be explained in terms of the known mechanisms of action of these drugs. This experimental model is therefore useful in assessing the effects of antimetabolites and nucleoside transport inhibitors in intact colonic mucosa.

55. Potentiation of the cytotoxicity of thymidylate synthase (TS) inhibitors by dipyridamole analogues with reduced alpha1-acid glycoprotein binding. Br. J. Cancer, Aug. 1, 1999; 80(11): 1738-46; N. J. Curtin, K. J. Bowman, R. N. Turner, B. Huang, P. J. Loughlin, A. H. Calvert, B. T. Golding, R. J. Griffin and D. R. Newell.

Dipyridamole has been shown to enhance the in vitro activity of antimetabolite anticancer drugs through the inhibition of nucleoside transport. However, the clinical potential of dipyridamole has not been realized because of the avid binding of the drug to the plasma protein alpha1-acid glycoprotein (AGP). Dipyridamole analogues that retain potent nucleoside transport inhibitory activity in the presence of AGP are described and their ability to enhance the growth inhibitory and cytotoxic effects of thymidylate synthase (TS) inhibitors has been evaluated. Three dipyridamole analogues (NU3026, NU3059 and NU3060) were shown to enhance the growth inhibitory activity of the TS inhibitor CB3717 and block thymidine rescue in L1210 cells. The extent of potentiation at a fixed analogue concentration (10 microM) was related to the potency of inhibition of thymidine uptake. A further analogue, NU3076, was identified, which was more potent than dipyridamole with a Ki value for inhibition of thymidine uptake of 0.1 microM compared to 0.28 microM for dipyridamole. In marked contrast to dipyridamole, inhibition of thymidine uptake by NU3076 was not significantly affected by the presence of AGP (5 mg ml(−1)). NU3076 and dipyridamole produced equivalent potentiation of the cytotoxicity of the non-classical antifolate TS inhibitor, nolatrexed, in L1210 cells with both compounds significantly reducing the LC90, by > threefold in the absence of salvageable thymidine. Thymidine rescue of L1210 cells from nolatrexed cytotoxicity was partially blocked by both 1 microM NU3076 and 1 microM dipyridamole. NU3076 also caused a significant potentiation of FU cytotoxicity in L1210 cells. These studies demonstrate that nucleoside transport inhibition can be maintained in the absence of AGP binding with the dipyridamole pharmacophore and that such analogues can enhance the cytotoxicity of TS inhibitor.

56. Characterization of a multidrug resistant human erythroleukemia cell line (K562) exhibiting spontaneous resistance to 1-beta-D-arabinofuranosylcytosine; Leukemia, S. Grant, A. Turner, P. Nelms and S. Yanovich; May 1, 1995; 9(5): 808-14.

We have assessed the response of a previously characterized multidrug resistant (MDR) human erythroleukemia cell line (K562R) to the nucleoside analog antimetabolite 1-beta-D-arabinofuranosylcytosine (ara-C). This cell line has been subjected to selection pressure by intermittent exposure to daunorubicin, but not ara-C, since its initial isolation. In comparison to the parental line (K562S), K562R were approximately 15-fold more resistant to ara-C as determined by 3H-dThd incorporation, MTT dye reduction and clonogenicity. Following a 4-h exposure to 10 microM ara-C, K562S accumulated approximately seven times more ara-CTP, and incorporated approximately 250% more ara-C into DNA than their resistant counterparts. The intracellular generation of ara-CTP was not significantly influenced by the cytidine deaminase inhibitor THU or the deoxycytidylate deaminase inhibitor dTHU (1 mM each) in either cell line. Rates of dephosphorylation of ara-CTP were equivalent in sensitive and resistant cells, as were intracellular levels of both ribonucleotide and deoxyribonucleotide triphosphates. However, K562R displayed a significant (ie 70%) reduction in the level of activity of the pyrimidine salvage pathway enzyme, deoxycytidine kinase (dCK), compared to K562S cells. In contrast to U937 leukemic cells, DNA extracted from K562S and K562R cells following exposure to 10 microM ara-C for 6 h did not exhibit the characteristic internucleosomal DNA cleavage on agarose gel electrophoresis typical of drug-induced apoptosis. Lastly, Northern analysis revealed equivalent levels of dCK message in the two cell lines. K562R represents an unusual example of a classical multidrug resistant human leukemic cell line exhibiting spontaneous cross-resistance to the antimetabolite ara-C, and may prove of value in attempts to understand the mechanism(s) by which human leukemic myeloblasts survive in vivo exposure to combination chemotherapeutic regimens containing drugs that are not classically associated with the multidrug resistance phenomenon.

57. Clofarabine: Bioenvision/ILEX; Curr Opin Investig Drugs, A. Sternberg; Dec. 1, 2003; 4(12): 1479-87;

Clofarabine is a purine nucleoside antimetabolite under development by Bioenvision (under license from the Southern Research Institute) and ILEX for the potential treatment of solid tumors, acute myelogenous leukemia, non-Hodgkin's lymphoma, and acute lymphoblastic and chronic lymphocytic leukemia. In September 2003, Bioenvision initiated a phase II trial in Europe in pediatric acute lymphoblastic leukemia, and in October 2003, ILEX submitted the first part of a rolling NDA to the FDA for the treatment of acute leukemia in children.

58. Corticosteroid responsive fludarabine pulmonary toxicity, Am. J. Clin. Oncol., Aug. 1, 2002; 25(4): 340-1, G. S. Stoica, H. E. Greenberg and L. J. Rossoff. Division of Pulmonary and Critical Care Medicine, Long Island Jewish Medical Center, New Hyde Park, N.Y. 11042-1101, U.S.A

Fludarabine monophosphate is a purine nucleoside antimetabolite with efficacy in the treatment of lymphoproliferative disorders and chronic lymphocytic leukemia. It is the 2-fluoro, 5′ phosphate derivative of 9-beta-D-arabinofuranosyl adenine (ara-A, vidarabine) and the mechanism of action is through inhibition of DNA synthesis and the cytolytic effects through the induction of endonuclease-independent apoptosis.

59. Cerebrospinal fluid pharmacokinetics and toxicology of intraventricular and intrathecal arabinosyl-5-azacytosine (fazarabine, NSC 281272) in the nonhuman primate. Invest New Drugs, May 1, 1993; 11(2-3): 135-40, R. L. Heideman, C. McCully, F. M. Balis and D. G. Poplack.

Arabinosyl-5-azacytosine (AAC), a new nucleoside antimetabolite, is broadly active in preclinical tumor screening evaluations. To assess the potential for intrathecal use of this drug, we studied the toxicity and pharmacokinetics of intrathecal and intraventricular administration in nonhuman primates. Four adult male rhesus monkeys were given single 10 mg intrathecal (n=1) or intraventricular (n=3) doses of AAC to determine its acute toxicity and pharmacokinetic parameters. An additional 3 animals were given four weekly 10 mg intrathecal doses to assess the systemic and neurologic toxicity associated with chronic administration. Disappearance from the cerebrospinal fluid (CSF) was biexponential, and CSF clearance was 0.2 ml/min, which exceeds the rate of CSF bulk flow by 5-fold. The peak CSF concentration and area under the concentration x time curve achieved with the intraventricular administration of 10 mg were one hundred, and fifty fold greater, respectively, than those achieved after an intravenous dose of 200 mg/kg (1500-2400 mg) in prior experiments. No clinically evident neurotoxicity was observed in either the single or the weekly×4 dose groups. A slight, transient CSF pleocytosis and increased CSF protein was observed. Systemic toxicity was limited to one animal in the weekly×4 dose group who demonstrated a mild and transient decrease in his peripheral leukocyte count unassociated with a change in his hematocrit or platelet count. These studies in nonhuman primates demonstrate a clear pharmacokinetic advantage for intrathecal vs systemic administration of AAC. This is demonstrated by a 50-fold greater CSF drug exposure with an intrathecal or intraventricular dose 1/200th of that which can be given systemically. (ABSTRACT TRUNCATED AT 250 WORDS).

60. Phase I trial and biochemical evaluation of tiazofurin administered on a weekly schedule, Sel. Cancer Ther., Mar. 1, 1990; 6(1): 51-61, T. J. Melink, G. Sarosy, A. R. Hanauske, J. L. Phillips, J. H. Bayne, M. R. Grever, H. N. Jayaram and D. D. Von Hoff.

Tiazofurin (2-B-D-Ribofuranosylthiazole-4-Carboxamide: NSC 286193) is a nucleoside antimetabolite that acts as a potent inhibitor of IMP dehydrogenase resulting in a guanine nucleotide deprivation. Recent in vivo biochemical observations in rats bearing hepatoma suggested a correlation between depletion of guanine nucleotides and antitumor effect. The present phase I trial utilized a weekly×3 bolus infusion schedule, repeated every 5 weeks. Biochemical measurements of GTP and dGTP were performed in patients at each dose level. Twelve patients received 16 courses of the drug in doses ranging from 1100 to 2050 mg/m2 weekly×3. The dose limiting toxicities were pericarditis and clinical symptoms suggestive of a more generalized serositis (chest and abdominal pain). Other toxicities included reversible elevations in CPK (MM band only) and SGOT, nausea, vomiting, and arthralgias. Neurotoxic effects were generally mild, including headaches, anxiety, and malaise. Only 1 of 6 patients evaluated for tiazofurin's biochemical activity showed a sustained depletion of guanine nucleotide pools. No antitumor activity was observed. The maximally tolerated dose of tiazofurin on this intermittent weekly×3 schedule was 1650 mg/m2. Toxicity and the overall lack of biochemical and biologic effect at clinically achievable doses may preclude further clinical evaluation of this drug on a weekly schedule. The toxicities observed in our study were similar to those reported for phase I investigations using a considerably higher dose intensity with daily×5 schedules.

61. Evaluation of purine and pyrimidine analogues in human tumor cells from patients with low-grade lymphoproliferative disorders using the FMCA, Eur. J. Haematol, May 1, 1999; 62(5): 293-9, Aleskog, R Larsson, M Hoglund, C Sundstrom, and J Kristensen.

The purine analogues fludarabine and cladribine (CdA) have recently become established to be effective treatment for low-grade non-Hodgkin's lymphoma (NHL). The pyrimidine nucleoside analogue cytarabine (AraC) has an important place in the treatment of acute leukemia, and gemcitabine is a new pyrimidine antimetabolite which has shown clinical activity against solid tumors. We have used the semiautomated fluorometric microculture cytotoxicity assay (FMCA), based on the measurement of fluorescence generated from cellular hydrolysis of fluorescein diacetate (FDA), to study these drugs. Eighty samples from 60 patients with low-grade NHL were studied. Fifty samples from patients with acute lymphoid leukemia (ALL) and 118 samples from patients with acute myeloid leukemia (AML) were included for comparison. The results indicate that the purine- and pyrimidine nucleoside analogues tested may be as active against low-grade NHL as against acute leukemia. In low-grade NHL, AraC seems to be even more active in comparison to CdA (p=<0.0001) and fludarabine (p=0.001). Untreated patients were more drug sensitive than previously treated patients. Gemcitabine showed the highest correlation with AraC (0.90) whereas CdA showed the highest correlation with fludarabine (0.84). Based on these results we propose that AraC and gemcitabine may have a role in the treatment of low-grade NHL.

62. Altered susceptibility of differentiating HL-60 cells to apoptosis induced by antitumor drugs, Leukemia, Feb. 1, 1994; 8(2): 281-8, G Del Bino, X. Li, F. Traganos, and Z. Darzynkiewicz.

It has been reported that human promyelocytic leukemic HL-60 cells which undergo differentiation fail to respond by apoptosis when treated with antitumor drugs, predominantly DNA topoisomerase inhibitors. Because S phase cells are selectively sensitive to these drugs, and during differentiation there is a reduction in the proportion of cells in S phase, the reported decrease in the number of apoptotic cells could simply be a reflection of the paucity of sensitive cells in these cultures. Using cytometric methods which allow apoptosis to be related to cell cycle position, we have compared the apoptotic response of HL-60 cells growing exponentially and induced to myeloid differentiation by dimethyl sulfoxide (DMSO). The cells were treated with: (i) the DNA topoisomerase I inhibitor camptothecin (CAM), which selectively triggers apoptosis or S phase cells; (ii) the nucleoside antimetabolite 5-azacytidine (AZC) and hyperthermia, both of which preferentially affects G1 cells; and (iii) gamma radiation, which causes apoptosis predominantly of G2+M cells. The cells exposed to 1.4% DMSO for 24 or 48 h were significantly more resistant to response by apoptosis, regardless of the nature of the agent and regardless of their position in the cell cycle. Thus, induction of differentiation lowers the cell's ability to respond to a variety of damaging agents by apoptosis and this effect is not correlated with cell cycle position. In addition, the difference in response was unrelated to expression of the apoptosis-modulating protein bcl-2, which appeared unchanged following 48 h exposure to DMSO. On the other hand, when the cells were pretreated with low concentrations of CAM or AZC, washed free of drug, and then treated with DMSO, the proportion of cells undergoing apoptosis was markedly increased, relative to drug-treated cells returned to DMSO-free medium. The present data may indicate that while the drug-induced damage screening mechanisms, which are linked to triggering apoptosis, may be more proficient in proliferating cells, the effectors of apoptosis are more expressed in cells undergoing differentiation. The data also suggest that the efficiency of chemotherapeutic agents or radiation may be reduced if a differentiating agent is used in combination therapy and is administered first. An enhancement of apoptosis, however, may be expected if the differentiating drug is administered in the reverse sequence.

63. Polarographic properties and potential carcinogenicity of some natural nucleosides and their synthetic analogues; Bioelectrochem Bioenerg, Feb. 1, 1999; 48(1): 129-34.; L. Novotny, A.Vachalkova, and A Piskala

The polarographic reduction and the index of potential carcinogenicity tg alpha determined polarographically in aprotic conditions and in the presence of alpha-lipoic acid of nine naturally occurring and synthetic pyrimidine and six synthetic 1,3,5-triazine (5-aza) nucleosides was compared to the reduction of eight synthetic 1,3,6-triazine (6-aza) nucleosides. Nucleosides are of interest because of their key role in the nucleic acid structure and because of the antimetabolite and cytotoxic/antileukemia properties of their synthetic analogues. It was shown that polarographic reduction of the studied compounds is achieved at gradually increased potentials in the order of 6-aza<5-aza<pyrimidine nucleosides. On other hand, the potential carcinogenicity of studied compounds increases usually in the order of pyrimidine<6-aza<<5-aza nucleoside. The only compounds with remarkable potential carcinogenicity identified at this study were those ones from the 5-aza (1,3,5-triazine) antimetabolite series-arabinosyl-5-azacytosine (0.275), 5-aza-cytidine (0.295) and 5-aza-uracil (0.400)-and 2,T-anhydrouridine (0.260). The relation of the data obtained to biological activity of nucleosides included in the study is discussed.

64. The Following Articles were taken from the book” Drug Resistance and Selectivity, Biochemical and Cellular Basis, Edited by Enrico Mihich, Roswell Park Memorial Cancer Institute, Buffalo, N.Y.; Academic Press, 1973, Pages 83-93, Chapter 3. CROSS-RESISTANCE AND COLLATERAL SENSITIVITY; Dorris J. Hutchinson and Franz A. Schmid”

### Examples of Purine Analogs-Drug Resistance:

Resistance to purine analogs was first described by Law and Boyle (1951) for 8-azaguanine in the L1210 mouse leukemia. As with all other chemo therapeutically useful antitumor drugs, neoplasms and other model systems resistant to 6-MP were described (Hutchison, 1963) shortly after the observed activity in experimental systems. On the biochemical level, the early studies on purine antagonist-resistant biological systems have been reviewed and summarized by Brockman (1963a,b) and Balis (1968). Un-doubtedly comparative studies on purine analog-resistant mutants and their wild-type parental lines have provided more information basic to the understanding of purine biosynthesis and metabolism than if only wild-type systems had been available.

Of the various mechanisms of resistance to purine analogs the most common was decreased or deleted enzymatic capacity to convert the analogs the analog to a nucleotide, the actual biologically active purine derivative. If, however, the pyrophosphorylase is functional but to a lesser extent than in the wild type (missed population of cells, less active enzymatic protein, etc.), some degree of response will be seen in the system to related compounds. Other mechanisms have been tabulated (Hutchison, 1963, 1965) and the more common are discussed in chapter 7.

Animal neoplasms resistant to 6-MP, thioguanine, and 6-methyl thio-purine ribonucleoside (6-MeMPR), which have been targets for chemotherapy studies and not summarized by us previously, are listed in Table VI.

Line L1210/MP(III) reported (Hutchison et al., 1962) to be collaterally sensitive to methotrexate, azaserine, and mitomycin C shows collateral sensitivity to the antibiotic, neocarzinostatin, to the alkylating agent, carbazilquinone, and to three new antifolates. It retained sensitivity to 6-MeMPR and ara-C.

Rutman et al. (1962) and Rutman (1964), who used a thioguanine resistant variant of L1210, reported collateral sensitivity to six alkylating agents but no change in response to cytoxan. Unlike L1210/MP(III), the sensitivity of L1210/TG/R to methotrexate and azaserine was not altered—it remained the same as the parental line.

Paterson and his group (Caldwell et al., 1967; Wang et al., 1967; Paterson and Wang, 1970) found that an Ehrlich ascites resistant to either 6-MP or thioguanine was partially cross-resistant to 6-MeMPR. Likewise a 6-MeMPR-resistant Ehrlich ascites was partially cross-resistant to 6-MP. These observations of what can be called partial cross-resistance (the drugs, 6-MP or 6-MeMPR, appear to be twice as effective in the treatment of the wild-type Ehrlich ascites or against the several resistant lines) fit with the biochemical data (Wang et al., 1967; Paterson and Wang, 1970).

The chemotherapeutic results were similar when two thioguanine-resistant Ehrlich ascites lines were treated with 6-MeM PR (Table VI).

The expression of partial cross-resistant of thioguanine- and 6-MP-resistant lines to 6-MP-resistant lines to 6-MeMPR can be attributed to the fact that these lines were able to convert, enzymatically, 6-MeMPR to 6-MeMPR-5′-monophosphate. However, the 6-MeMPR-resistant line was capable of enzymatically forming some 6-MP ribonucleotide. The chemotherapeutic data and relative biochemical activities of the various purine analogs resistant to Ehrlich ascites are compatible.

Examples of Pyrimidine Analogs-Drug Resistance

Resistance to a fluoropyrimidine was first reported by Heidelberger et al. (1958). Many animal neoplasms and other biological systems that are resistant to fluoropyrimidines have been described since then (Hutchison, 1963, 1965). In general, they were all cross-resistant to other fluoro-pyrimidine analogs but retained their sensitivity to antifolates, purine analogs, and alkylating agents.

Rutman et al. (1962) and Rutman (1964) conducted extensive chemotherapy experiments using a 5-fluorouracil-resistant P815 neoplasm (P815-E176) with the specific purpose of searching for collateral sensitivity to alkylating agents and antimetabolites. Results of these tests are summarized in Table VII. Increased sensitivity was observed to five alkylating agents, three of which were newly synthesized Compounds, but there was cross-resistance to triethylenethiophosphoramide (thio-TEPA) and no change in response to the antimetabolites, cytoxan and nitrogen mustard (HN2). Rutman (1964) concluded that collateral sentivitity need not arise as an “all or none” phenomenon, i.e., there was no predictable pattern of response to a group of alkylating agents.

With continued interest in pyrimidine analogs and the resulting synthesis of ara-C (Walwick et al., 1959) and 1β-D-arabinofuranosyl-5-fluorocytosine (ara-FC) (Fox et al., 1966), Burchenal et al. (1966) found that a line of P815 resistant to 5-fluorouracil (FU) retained the same sensitivity as the P815 parent line to both cytosine analogs.

Heidelberger and Anderson (1964) described the antitumor activity of 5-trifluoromethyl-2′-deoxyuridine (F₃TdR) against several animal neoplasms including an Ehrlich ascites resistant to 5-fluorodeoxyuridine (FUdR). The resistant neoplasm was found to be cross-resistant to F₃TdR. Based on information relating to the mode of action of FUdR and bio-chemical alterations in the FUdR-resistant Ehrlich ascites, it was concluded that the inhibition of thymidylate synthetase may be more important in the mechanism of tumor inhibition than in the incorporation of the analog into DNA.

The mechanism of resistance to fluorinated pyrimidines and the history of their development have been summarized (Hutchison, 1963, 1965). Recent observations have added little to earlier results.

An interesting study was described by Blair and Hall (1969) in which they followed the development of resistance in the Ehrlich ascites to 6-azauracil and 6-azauridine. However, they were unable to correlate either decreased or increased activities of uridine kinase or uridine phosphorylase with development of resistance. One 6-azauracil-resistant line was cross-resistant to 6-azauridine and collaterally sensitive to FU (Table VII).

As mentioned, the attention of several laboratories was turned to cytosine derivatives. Vesely and his colleagues (1968, 1970) characterized two lines of the AKR mouse leukemia—one resistant to 5-azacytidine (AKR/r-AzCR) and the other to 5-aza-2′-deoxycytidine (AKR/r-AzCdR). The subline resistant to 5-azacytidine (AzCR) was cross-resistant to 5-aza-2′-deoxycytidine (AzCdR) and ara-C. This can be explained on the basis of a deletion or loss of the enzyme deoxycytidine kinase. On the other hand the subline resistant to AzCdR was sensitive to AzCR but cross-resistant to ara-C. In this line uridine kinase functioned Normally and ribonucleic acid (RNA) polymerase activity increased. The cross-resistance to ara-C is probably due to the partial loss of deoxycytidine kinase.

1-β-D-Arabinofuranosylcytosine was synthesized in 1959 by the Upjohn group (Walwick et al., 1959). Smith (1967) reviewed in detail the background and development of this interesting pyrimidine analog. Numerous biochemical studies have been carried out that used ara-C as the antimetabolite and others that used ara-C-resistant cell cultures and animal neoplasms. Clinical results with ara-C in cases of acute lymphocytic and acute granulocytic leukemias have been favorable (Howard et al., 1966; Ellison et al., 1968).

Wodinsky and Kensler (1964) reported the selection of an ara-C-resistance subline of the L1210 mouse leukemia. Although resistance was not achieved in three transplant generations, it was complete at the tenth generation. A selected group of twenty-four drugs with diverse modes of action was tested against L1210 and L1210/ara-C. As indicated in Table VII cross-resistance was not observed to any compound and likewise no increase in sensitivity. That there was no cross-resistance to alkylating agents nitrosoureas, purine and pyrimidine analogs, nor antifolates was thought to be significant in regard to clinical use. A report (Evans et al., 1964) had shown that a line of L1210/C95 resistant to methotrexate, 6-MP and cytoxan was sensitive to ara-C (Table V).

Dixon and Adamson (1965) stated that ara_C-resistant variants of the L1210 mouse leukemia could be selected in one generation. This observation is quite different from that of Wodinsky and Kensler (1964). One of the ara-C-resistant variants was compared with L1210 in respect to response to several known antileukemic drugs (Table VII). No cross-resistance nor collateral sensitivity was observed.

Several other ara-C-resistant neoplasms have been selected (Table VII), and no cross-resistance has been observed to compounds such as hydroxy urea, guanazole, pyridine-2-carboxaldehyde thiosemicarhazone (TSC), bis(guanyihydrazones), and carbazilquinone. Line L51787/ara-C (Schmid and Hutchison, 1971b) was collaterally sensitive to L-asparaginase which is ill keeping with the noted increased sensitivity of L5178Y/CA55 to L-asparaginase (Schmid arid Hutchison, 1971b)

The ara-C was active against hydroxyurea-, vincristinetine-, VLB-, TSC-, cortisone- and methyiglyoxal bis(guanylhydrazone) (MGGH)-resistant mouse leukemias (see Tables XXIII and XXIV). An L1210 line resistant to MGGH was collaterally sensitive to ara-C (see Table XXIII).

The mechanisms of resistance to ara-C have been reviewed and summarized by Uchida and Kreis (1969) and by Drahovsky and Kreis (1970) and are also discussed in Chapter 7. However, the overall observation of lack of cross-resistance in ara-C-resistant tumors and, conversely, no cross-resistance development in a variety of neoplasms resistant to other effective antileukemic drugs places ara-C in a favorable position as an effective chemotherapeutic agent at many stages or steps during the use of cyclic chemotherapy in the clinic.

TABLE VI
Animal Neoplasms Resistant to Purine Analogs
Response to chemotherapeutic drugs^{a}

| | | same as | | collaterally | |
|---|---|---|---|---|---|
| Drug | Neoplasm | parent line | Cross-resistant | sensitive | References |
| 6-Mercaptopurine | Sarcoma 180 | Ara-C | — | Quinaspar | Evans et., 1964 |
| | L1210/MP(III) | 6-MeMPR | | Chlorasquin | Bennett et., 1965 |
| | | Ara-C | | Methasquin | Bradner and Hutchison, |
| | | | | Carbazilquinone | 1966 |
| | | | | Neocarzinostatin | Hutchison, 1968a |
| | | | | | Schmid and |
| | | | | | Hutchison, 1971c |
| | Ehrlich ascites | — | 6-MeMPR^{b} | — | Wang et al., 1967 |
| | (EAC-R1) | | | | Paterson and Wang, 1970 |
| | L1210/MP | DIC | NSC-82196 | — | Wodinsky et al., 1968 |
| Thioguanine | L1210 TG/R | Methotrexate | 5-Fluorouracil | Nitrogen mustard | Rutman et al., 1962 |
| | | azaserine | A-139 | Thio-TEPA | |
| | | Cytoxan | | L-PAM | |
| | | | | No. 30020 | |
| | | | | No 30024 | |
| | | | | No. 30025 | |
| | | | | No. 30035 | |
| | Ehrlich ascites | — | 6-MeMPR^{b} | — | Paterson and Wang, 1970 |
| | (ETGR1) | | | | |
| | (ETGR11) | — | 6-Mercaptopurine | — | Paterson and Wang, 1970 |
| 6-Methyl thiopurine | Ehrlich ascites | — | Formycin | — | Caldwell et al., 1967 |
| ribonucleoside | (EAC-R2) | | 6-Mercaptopurine^{b} | | Wang et al.., 1967 |
| | | | | | Paterson and Wang, 1970 |

TABLE VII
ANIMAL NEOPLASMS RESISTANT TO PYRIMIDINE ANALOGS
Response to chemotherapeutic drugs^{a}

| | | Same as | | | |
|---|---|---|---|---|---|
| Drug | Neoplasm | parent line^{b} | Cross-resistant | Collaterally | Reference |
| 5-Fluorouracil | P815-E176 | Methotrexate | Thio-TEPA | L-PAM | Rutman et al., 1962 |
| | | Azaserine | | A-139 | Rutman, 1964 |
| | | Thioguanine | | No. 30020 | |
| | | Cytoxan | | No. 30025 | |
| | | Nitrogen mustard | | No. 30035 | |
| | | No. 30024 | | | |
| | P815/FU | Ara-C | — | — | Burchenal et al., 1966 |
| | | Ara-FC | | | |
| 5-Fluorodeoxy- | | Ehrlich ascites - | 5-Fluorouracil | | |
| uridine | | | F₃TdR | — | Heidelberger and Anderson, |
| | | | | | 1964 |
| 6-Azauracil | | Ehrlich ascites - | 6-Azauridine | | 5-Fluorouracil |
| | | (AZU 1) | | | Blair and Hall, 1969 |
| 5-Azacytidine | | AKR/r-AzCR - | AzCdR | — | Vesely et al., 1968 |
| | | | Ara-C | | Vesely et al., 1970 |
| 5-Aza-2′-deoxy- | | AKR-r-AzCdr AzCR | Ara-C | — | Vesely et al., 1968, |
| cytidine | | | | | 1970 |
| 1β-D-Arabino- | L1210 | Alkylating agents (3)- | | — | Wodinsky and Kensler, |
| furanosylcytosine | | Nitrosoureas (3) | | | 1964 |
| | | Phthalanilides (3) | | | |
| | | Purine antagonists (3) | | | |
| | | Pyrimidine | | | |
| | | Antagonists (3) | | | |
| | | Antifolates (2) | | | |
| | | Miscellaneous | | | |
| | | Compounds (7) | | | |
| | L1210/CA | Hydroxyurea | — | — | Dixon and Adamson 1965 |
| | | Hydroxycarbamic | | | |
| | | Acid ethyl ester | | | |
| | | DCM | | | |
| | | MGGH | | | |
| | | BCNU | | | |
| | P815 | Hudroxyurea | — | | -Kreis and Hutchison, |
| | | | | | 1969 |
| | L1210-ara-C | MGGH | DDUG | — | -Mihich et al., 1970 |
| | | L1210/ara-C | Hydroxyurea | — | -Schabel et al., 1971 |
| | | | Guanazole TSC | | |
| | L5178Y/ara-C | Methotrexate | — | | Schmid and Hutchinson, |
| | | | L-asparaginase | | 1971 b, c |
| | | Cytoxan | | | |
| | | Carbazilquinone | | | |

Notes for Tables:
^{a}α Ara-C-1β-D-arabionfuranosylcytosine; are-FC-1β-arabionfuranosyl-5-fluorocytosine; No. 30024-1-bis(β-chlroethyl)amino-2-dimethyl aminoethane; DCM-3′,5′-dichloromethopterin; MGGH-methylglyoxal bis(guanylhydrazone); DDUG-4′,4′-diacetyldipenylurea bis(guanylhydrazone); BCNU-1,3-bis(2-chloroethyl)-1-nitrosourea; TSC-pyridine-2-carboxaldehyde thiosemicarbazone; thio-TEPA-triethylenethiophosphoramide; F3TdR-5-trifluoromethyl-2′-deoxyuridine; AzCdR-5-aza-2′-deoxycytidine; L-PAM-L-phenylalanine mustard methanol; A-139-2,5-bis(1-aziridinyl)-3,6-bis(2-methoxyethoxy)-p-benzoquinone; No. 30020-6-hydroxy-9-{3-[bis(2″-chloroethyl)amino}purine; No. 30025-1bis(β-chloroethyl)amino-4-aminopentane; No. 30035-1-bis(β-chloroethyl)amino-2-aminoethane).
^{b}Numbers in parentheses indicate number of compounds in each group.

### BRIEF DESCRIPTION OF THE DRAWINGS

FIG. 1 depicts 2′-FANA-Guanosine (anti conformation);

FIG. 2 depicts 2′-Ara-guanosine dG (syn conformation);

FIG. 3 depicts (C-2′-Endo Conformation);

FIG. 4 depicts (C-3′-Endo Conformation);

FIG. 5 depicts 2′-Ara-Omethyl-Uridine-free base prefers anti conformation;

FIG. 6 depicts 2′-O-methyl locking of purine ring (2′-Ara-O-methyl-guanosine-free base);

FIG. 7a depicts Parallel with dG's (FIG. 7: G-Quadruplexes);

FIG. 7b depicts antiparallel with G (FIG. 7: G-Quadruplexes);

FIG. 8a depicts HIV Inhibitor-14-mer-Consisting of 2′-Ara-O-Methyl Bases-3D representation;

FIG. 8b depicts HIV Inhibitor-14-mer-Consisting of 2′-Ara-O-Methyl Bases-formula sketch;

FIG. 9a depicts Thrombin 1-Oligonucleotide-Consisting of 2′-Ara-Omethyl-Bases 3 D representation;

FIG. 9b depicts Thrombin 1-Oligonucleotide-Consisting of 2′-Ara-Omethyl-Bases formula sketch;

FIG. 10a depicts HIV Inhibitor-Oligonucleotide consisting of DNA bases-3 D representation;

FIG. 10b depicts HIV Inhibitor-Oligonucleotide consisting of DNA bases-formula sketch;

FIG. 11 depicts Cyclic Array of four guanosines sequence;

FIG. 12 depicts Ara-nucleosideoligo;

FIG. 13 depicts 1-H NMR of2′-OMethyl-Ara-Guanosine (structure 19a);

FIG. 14 depicts 1-H NMR of 2′-OMethyl-Ara-Uridine (structure 24);

FIG. 15 depicts 1-H NMR of 2′-OMethyl-Ara-cytidine (structure 29);

FIG. 16 depicts 1-H NMR of 5′-DMT-2′-O-Methyl-Ara-Guanosine-n-ibu (structure 19);

FIG. 17 depicts 1-H NMR of 5′-DMT-2′-O-Methyl-Ara-Uridine (structure 25);

FIG. 18 depicts 1-H NMR of 5′-DMT-2′-O-Methyl-Ara-Cytidine-n-bz (structure 31);

FIG. 19 depicts 13 C NMR of 5′-DMT-2′-OMethyl-Ara-G-n-ibu (structure 19);

FIG. 20 depicts 13 C NMR of 5′-DMT-2′-OMethyl-Ara-Uridine (structure 25);

FIG. 21 depicts 13 C NMR of 5′-DMT-2′-OMethyl-Cytidine-n-ibu (structure 31);

FIG. 22 depicts ESI/MS of 2′-OMethyl-Ara-Guanosine-n-ibu (structure 19);

FIG. 23 depicts ESI/MS of 5′-DMT-2′-OMethyl-Ara-uridine (structure 25);

FIG. 24 depicts ESI/MS of 5′-DMT-2′-OMethyl-Ara-Cytidine-n-bz (structure 31);

FIG. 25 depicts ESI/MS of 5′-DMT-2′-OMethyl-ara-Guanosine-n-ibu (structure 20);

FIG. 26 depicts 1 H NMR of 5′-DMT-2′-OMethyl-ribo guanosine-n-ibu;

FIG. 27 depicts 1 H NMR of 5′-DMT-2′-OMethyl-Ara-Guanosine-(structure 19) (Expanded from 5-10 ppm);

FIG. 28 depicts 1 H NMR of 5′-DMT-2′-Omethyl-ribo-Uridine;

FIG. 29 depicts 1 H NMR of 2′-Omethyl-ribo-Guanosine;

FIG. 30 depicts 1 H NMR of DMT-2′-Omethyl-Ara-G-n-ibu-3′-cyanoethyl phosphoramidites (structure 20);

FIG. 31 depicts 1 H NMR of DMT-2′-Omethyl-Ara-U-3′-cyanoethyl phosphoramidite (structure 26);

FIG. 32 depicts 31 P NMR of DMT-2′-Omethyl-Ara-G-3′-cyanoethyl phosphoramidite (structure 20);

FIG. 33 depicts 31 P NMR of DMT-2′-Omethyl-Ara-U-3′-cyanoethyl phosphoramidite (structure 26);

FIG. 34 depicts Sequence Name: HIV-Inhibitor-14 mer

Sequence: (5′-3′)

aomGaomGaomGaomGaomUaomGaomGaomGaomUaomGaomGaomUaomGaomG6 and shows the migration time sequence (from left to right): 11.554, 11.877, 12.217, 12.521, 12.817, 12.967, 13.338, 13.658, 14.517, 15.488, 17.275, 17.992

Lot #071008-01;

FIG. 35 depicts Sequence Name: Thrombin-1

Sequence: (5′-3′)

aomGaomGaomUaomUaomGaomGaomUaomGaomUaomGaomGaomUaomUaomGG and shows the migration time sequence (from left to right): and shows the migration time sequence (from left to right): 13.571, 14.117, 14.700, 15.249

Lot #071008-02;

FIG. 36 depicts UV Spectrum and ratio of 250/260 and 260/280

Sequence Name: HIV-Inhibitor-14 mer

Sequence: (5′-3′)

aomGaomGaomGaomGaomUaomGaomGaomGaomUaomGaomGaomUaomGaomG6

Lot #071008-01;

FIG. 37 depicts UV Spectrum and ratio of 250/260 and 260/280

Sequence Name: Thrombin-1

Sequence: (5′-3′)

aomGaomGaomUaomUaomGaomGaomUaomGaomUaomGaomGaomUaomUaomGG

Lot #071008-02;

FIG. 38 depicts Trityl Histogram during Oligo nucleotide synthesis: Sequence Name: HIV-Inhibitor-14 mer: Sequence: (5′-3′)

:aomGaomGaomGaomGaomUaomGaomGaomGaomUaomGaomGaomUaomGaomG6, where 6: dT, aom: Ara-2′-O-Methyl;

FIG. 39 depicts Trityl Histogram during Oligo nucleotide synthesis: Sequence Name: Thrombin-1 Sequence: (5′-3′)

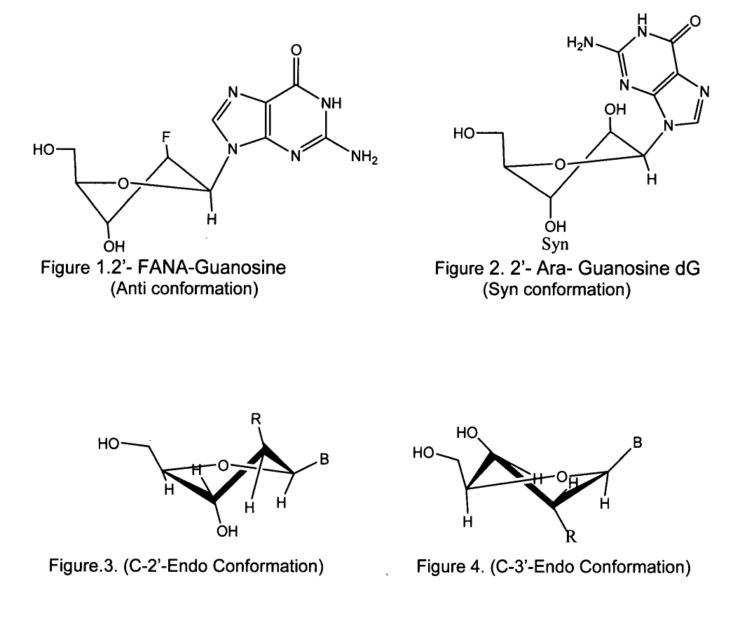

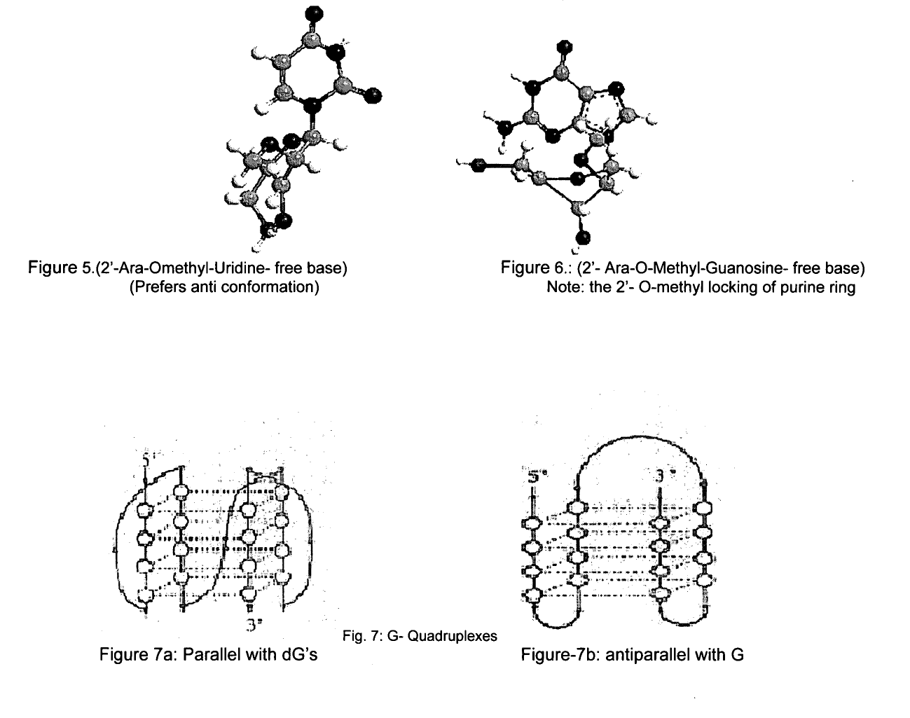

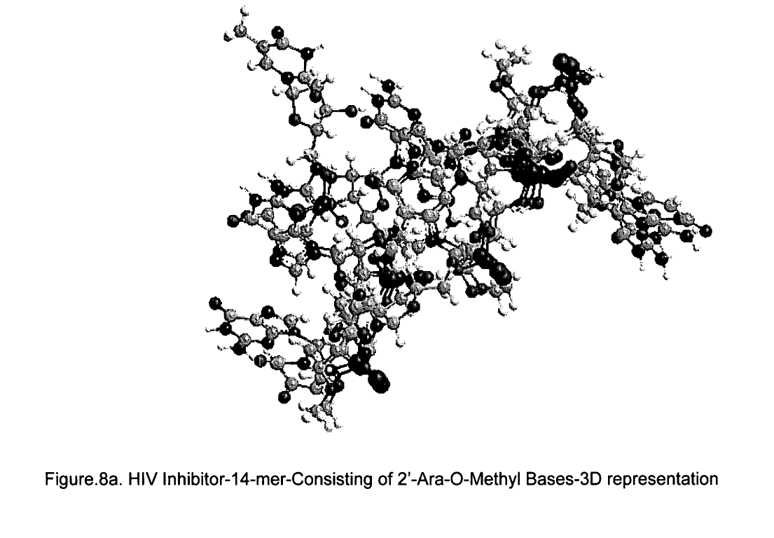

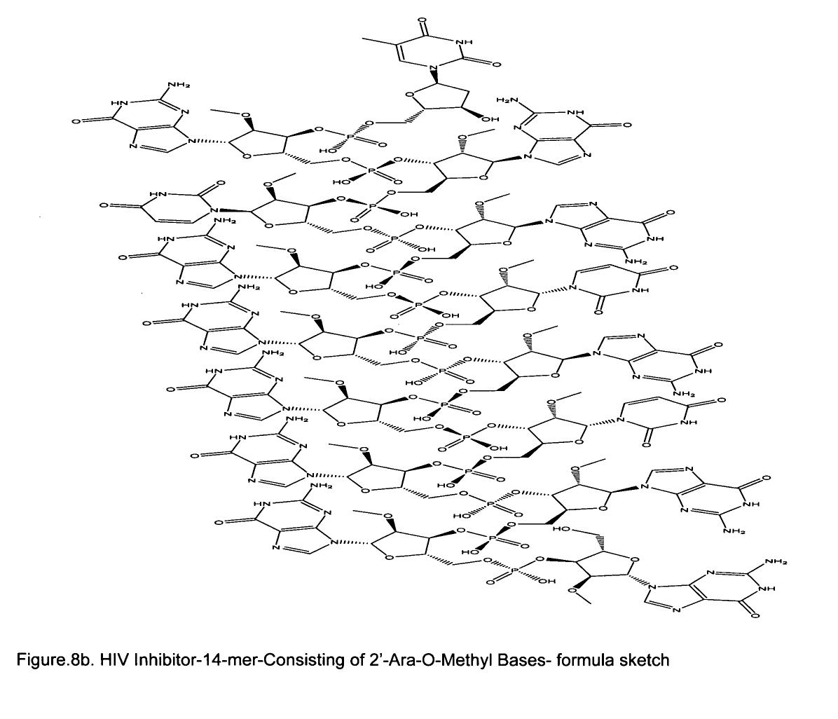

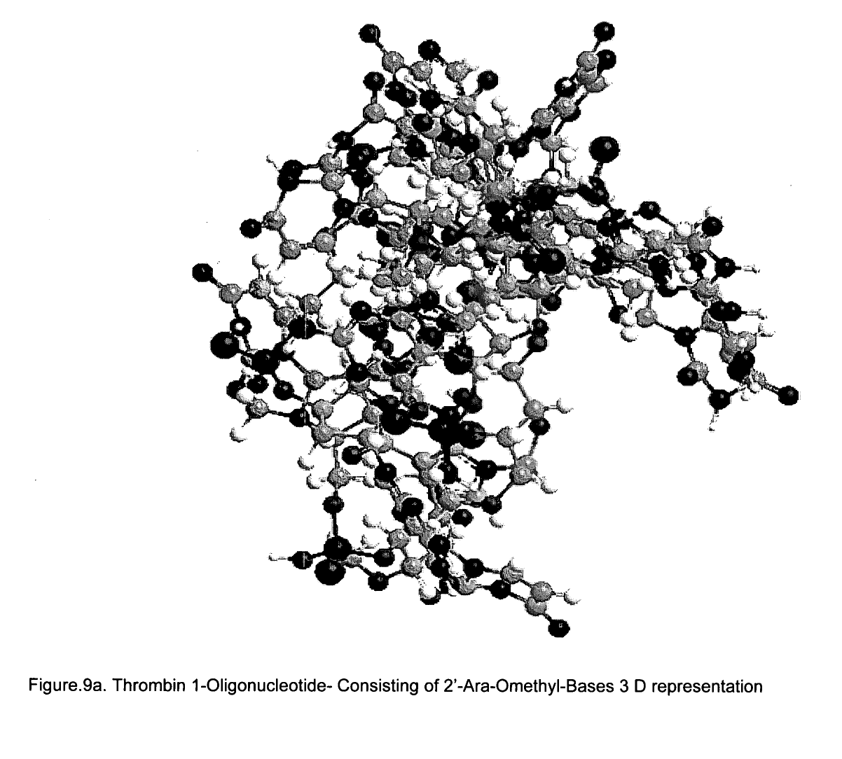

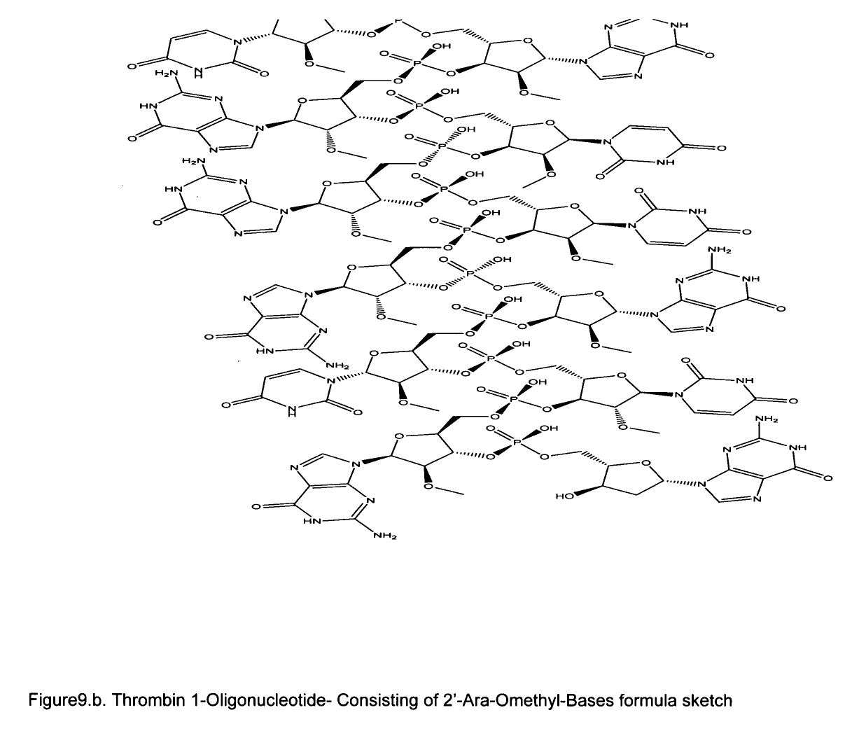

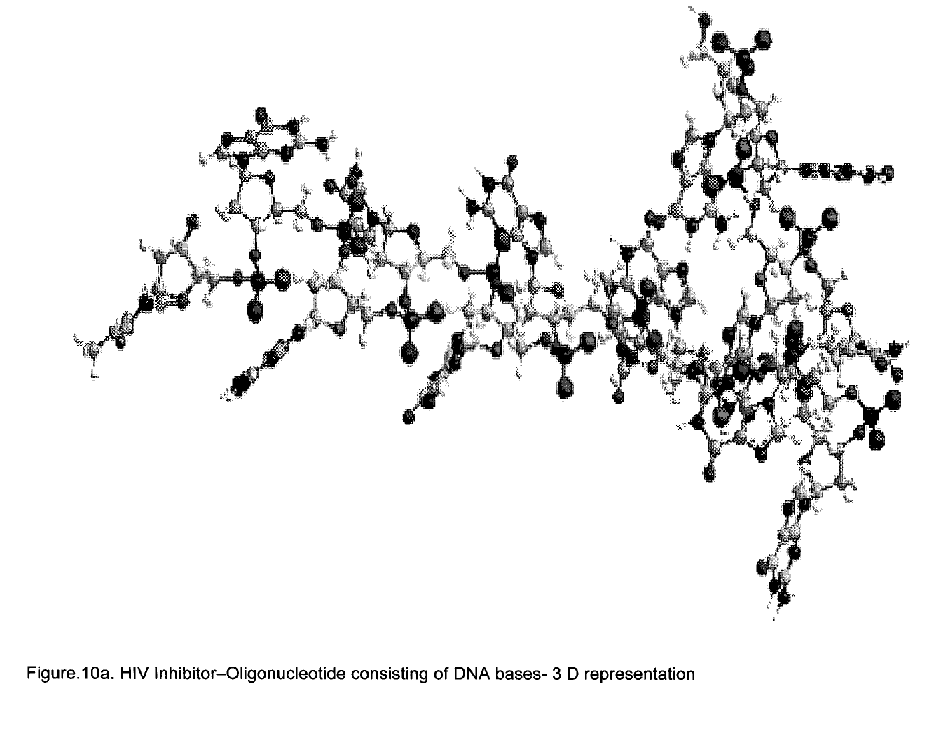

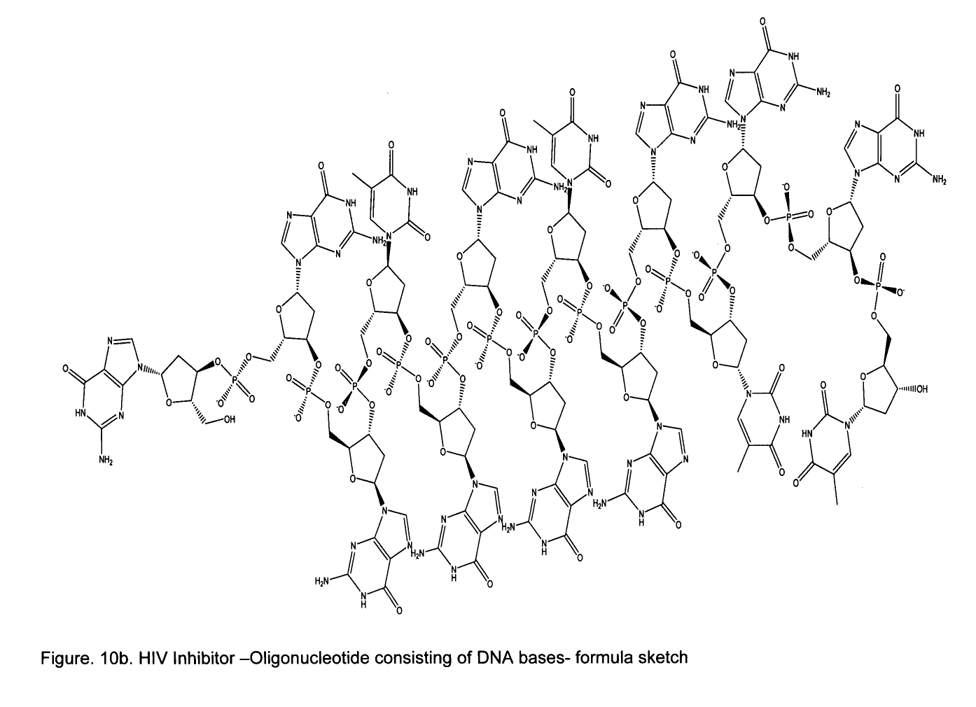

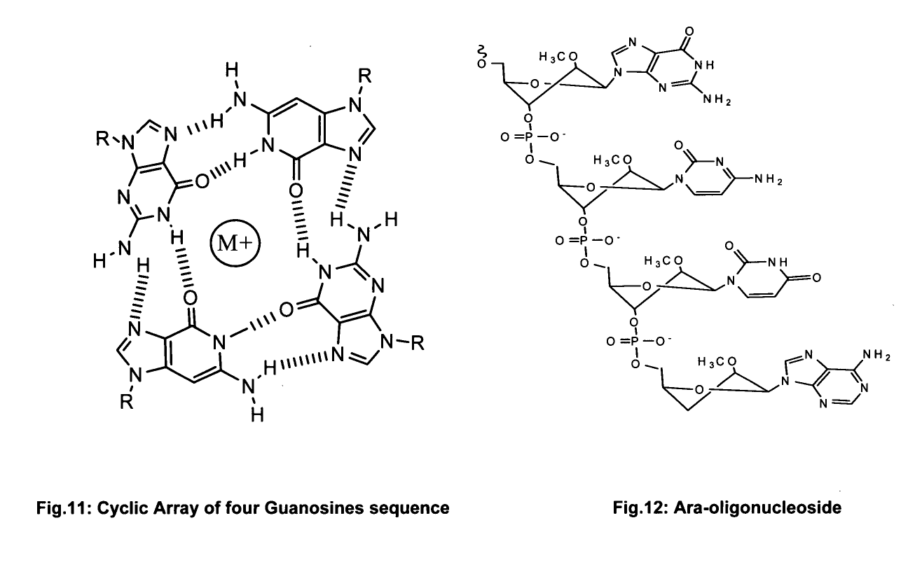

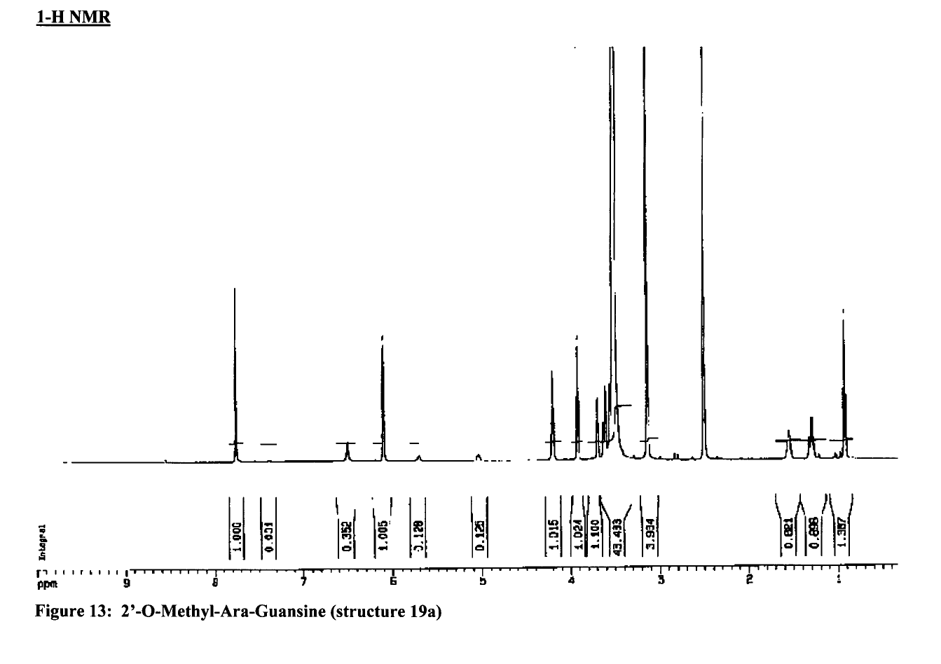

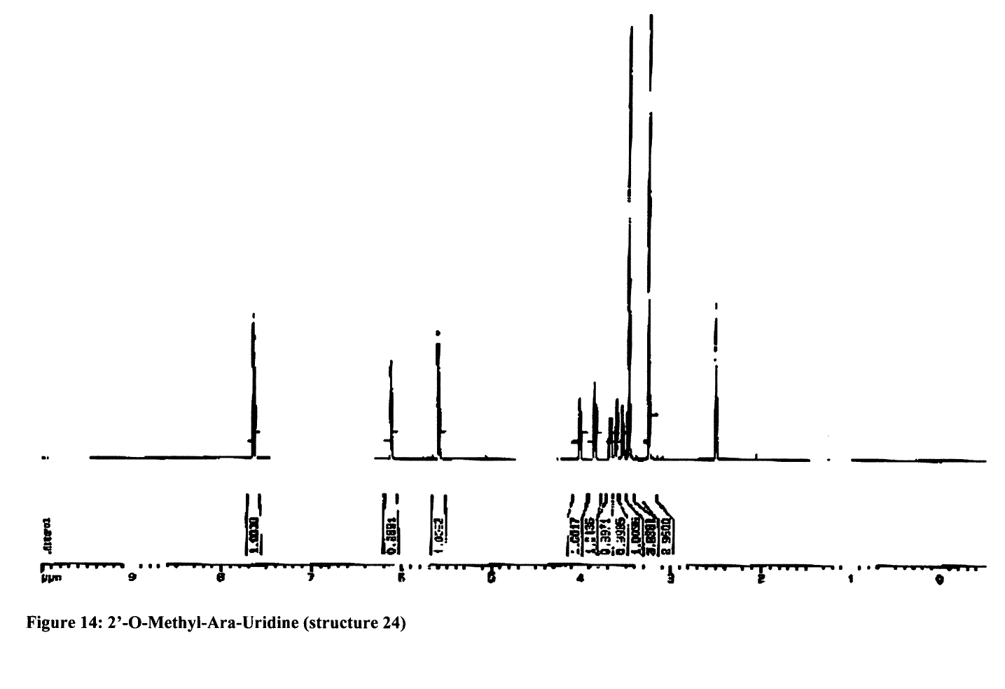

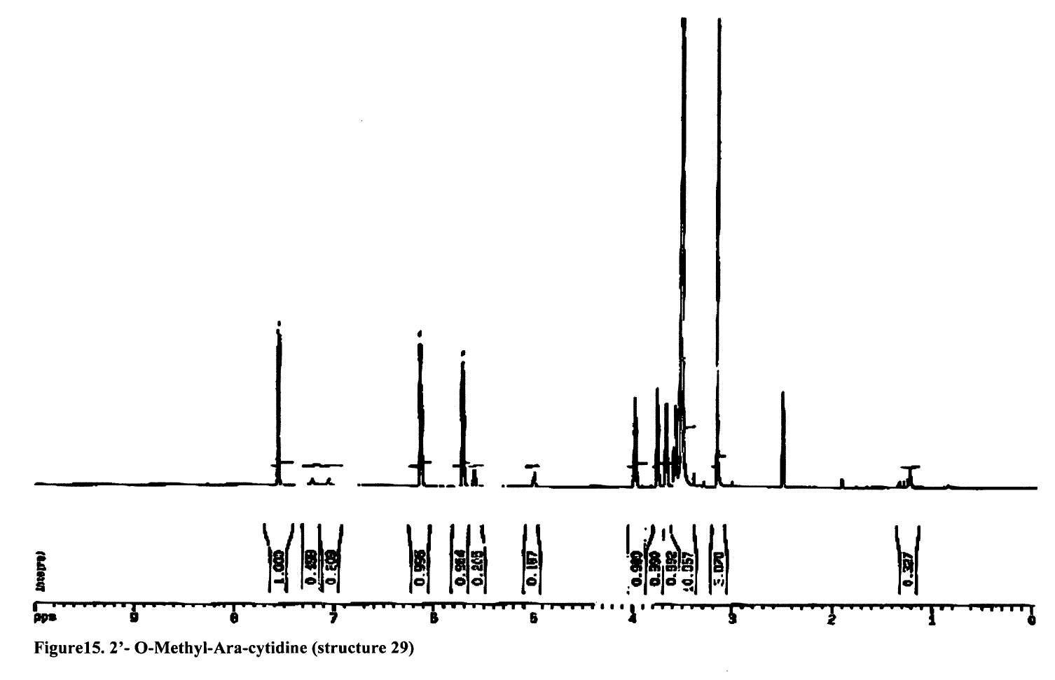

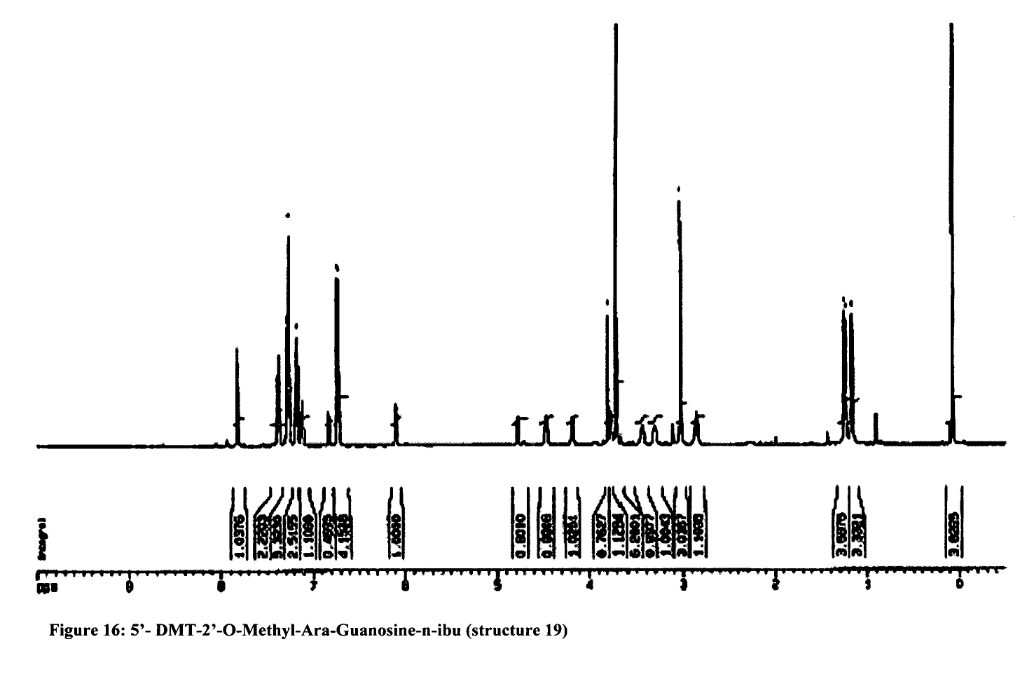

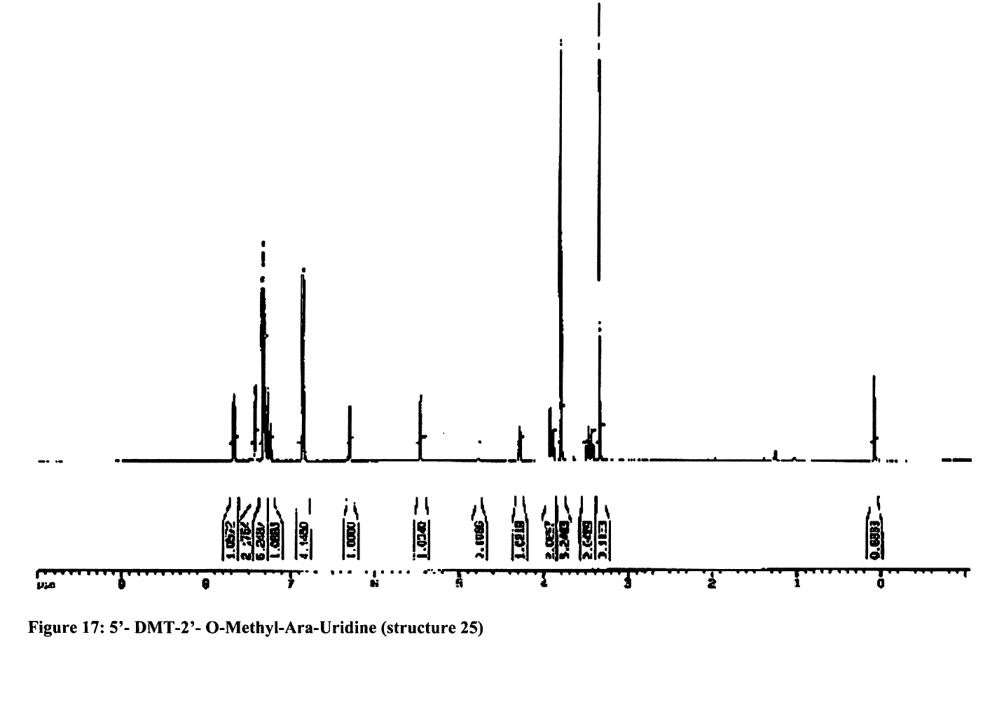

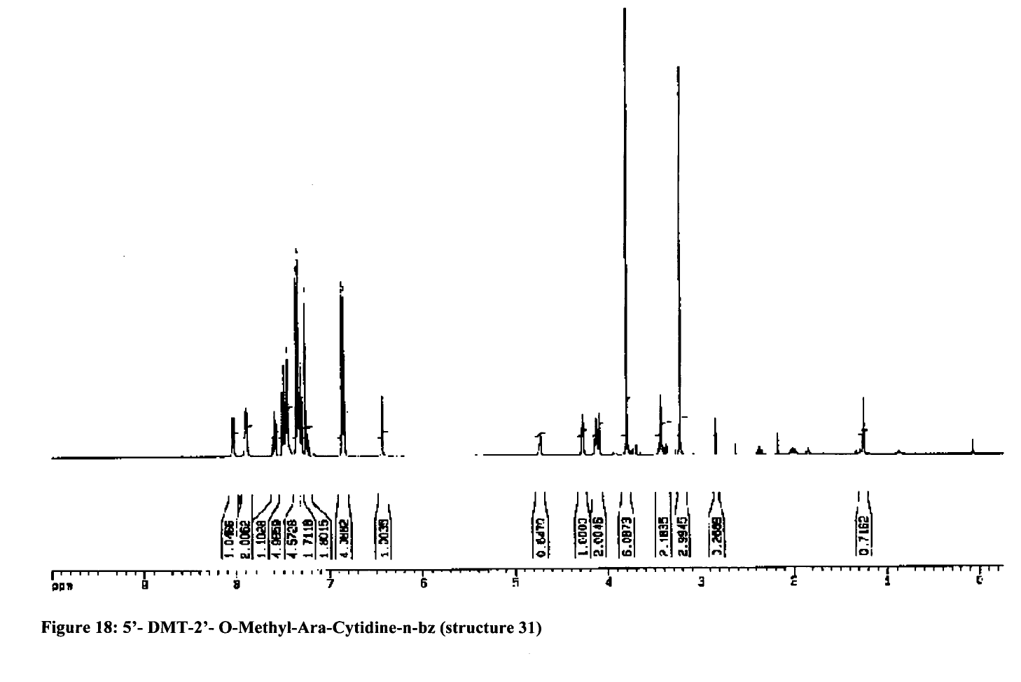

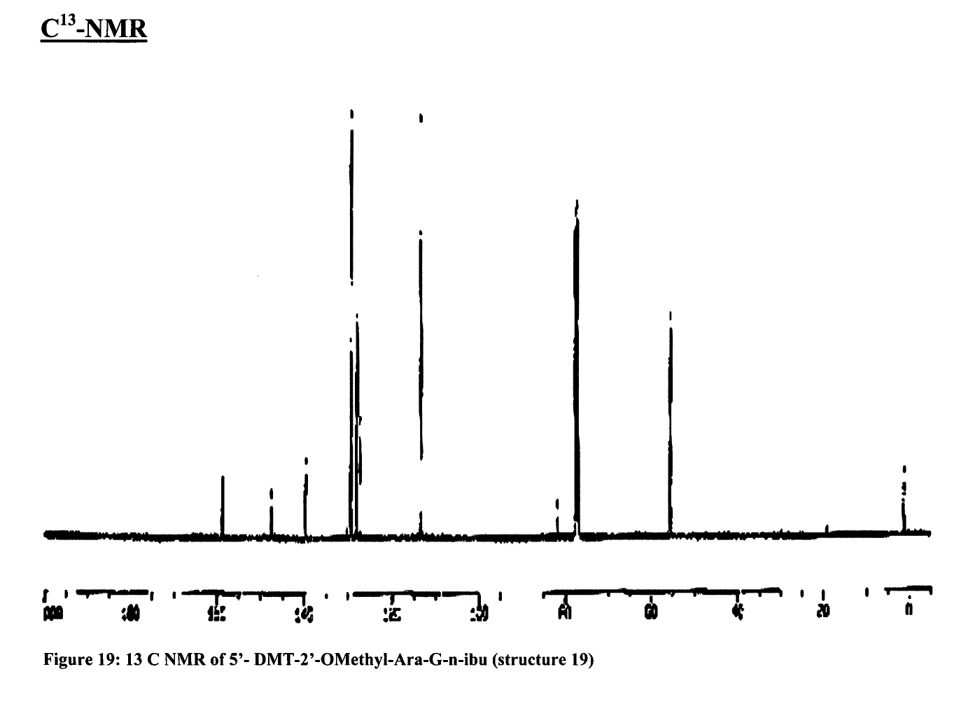

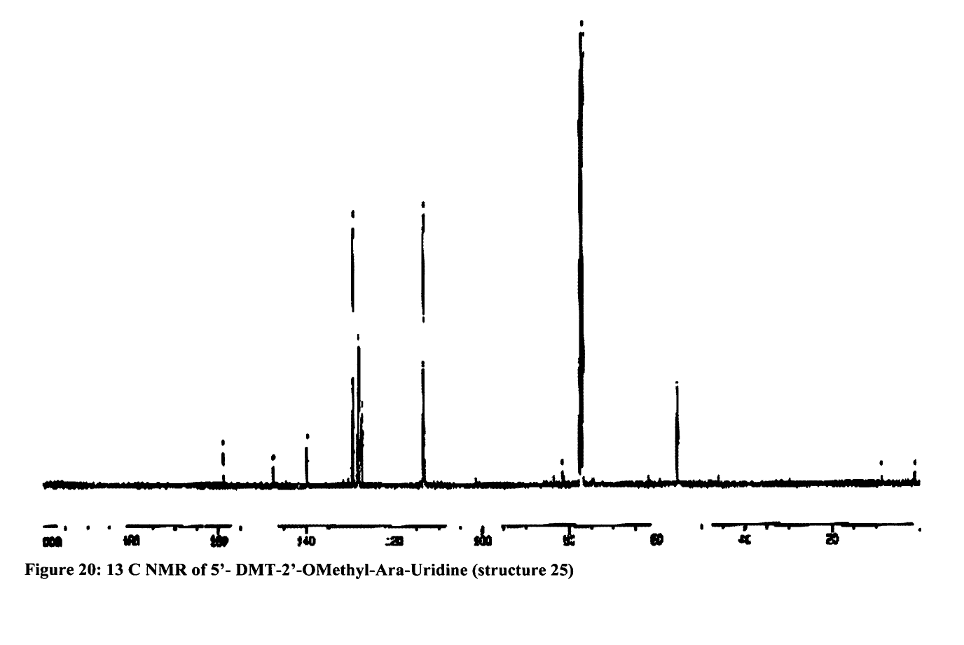

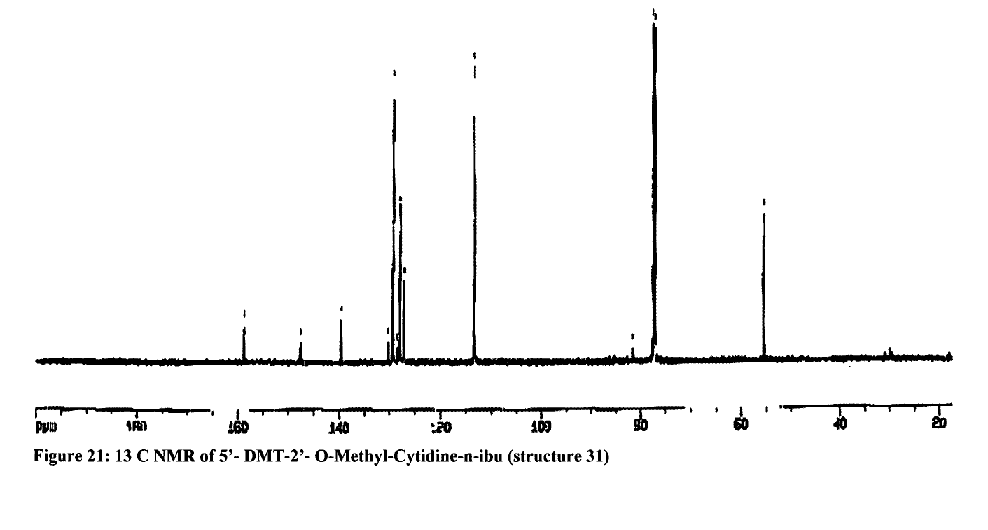

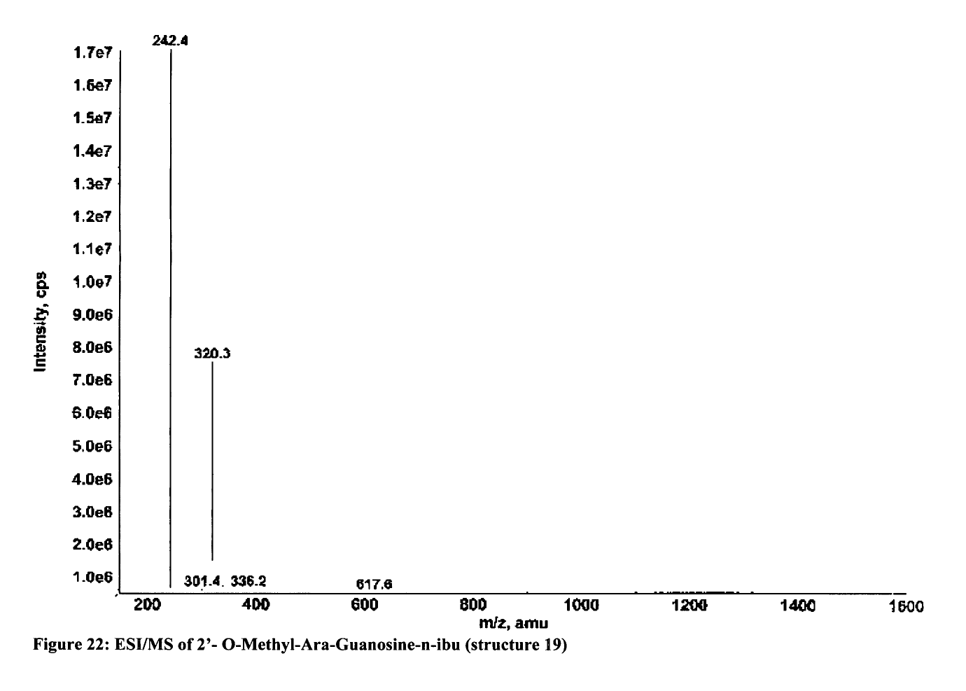

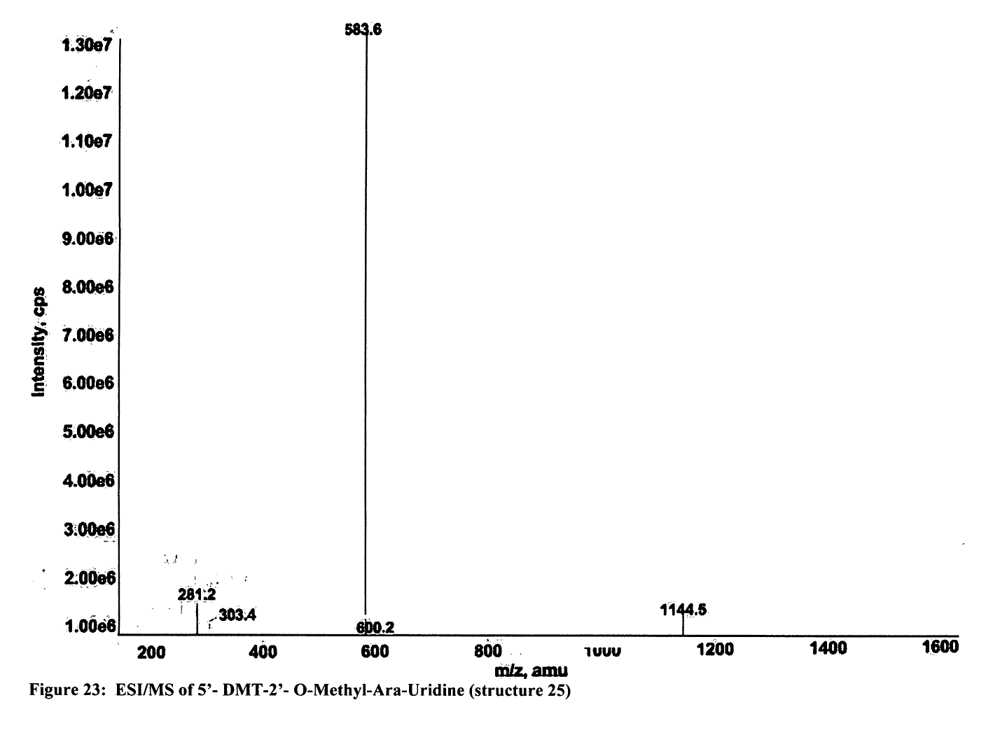

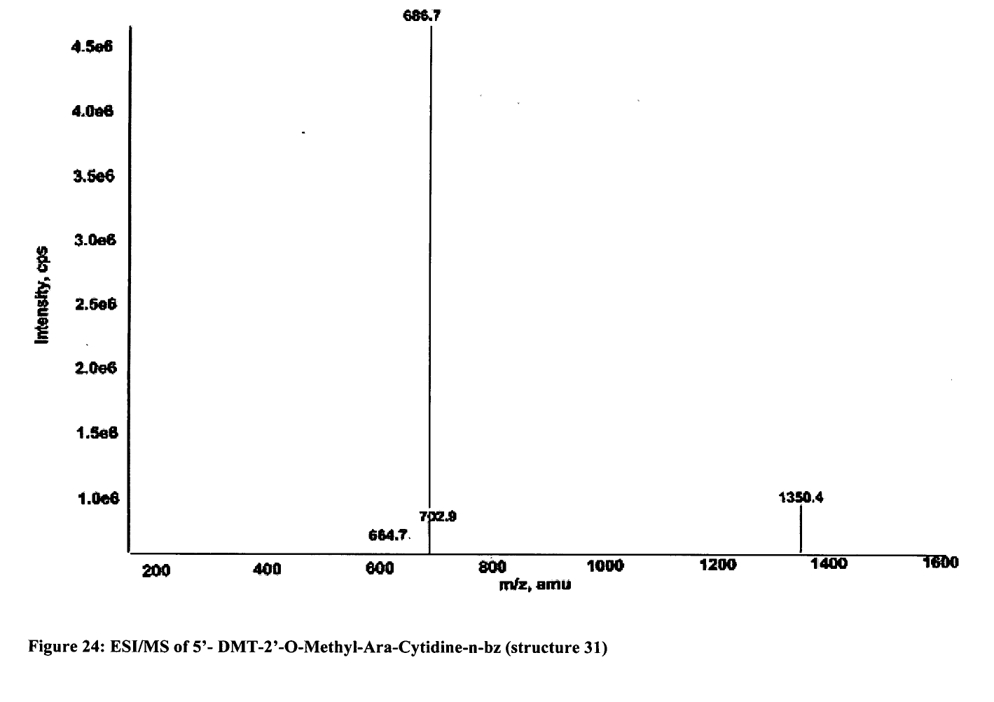

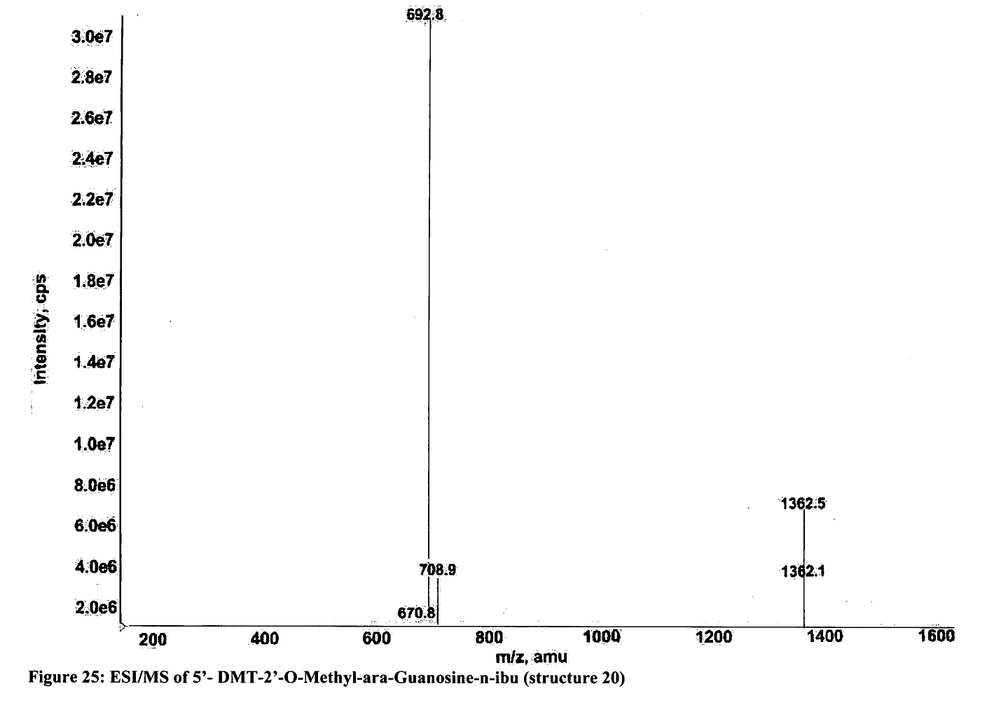

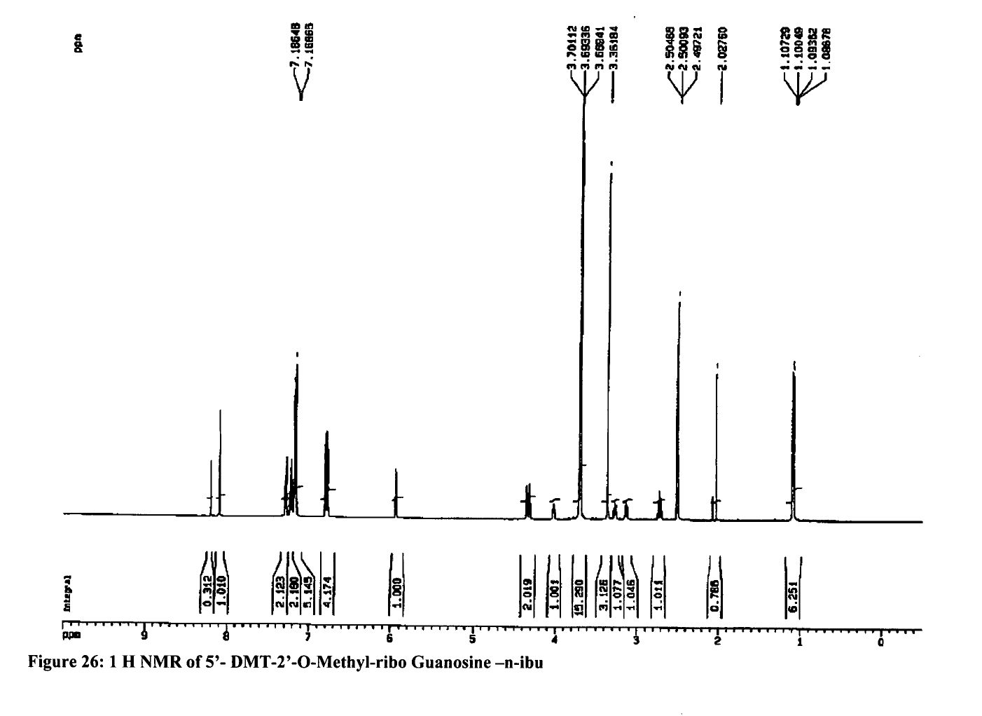

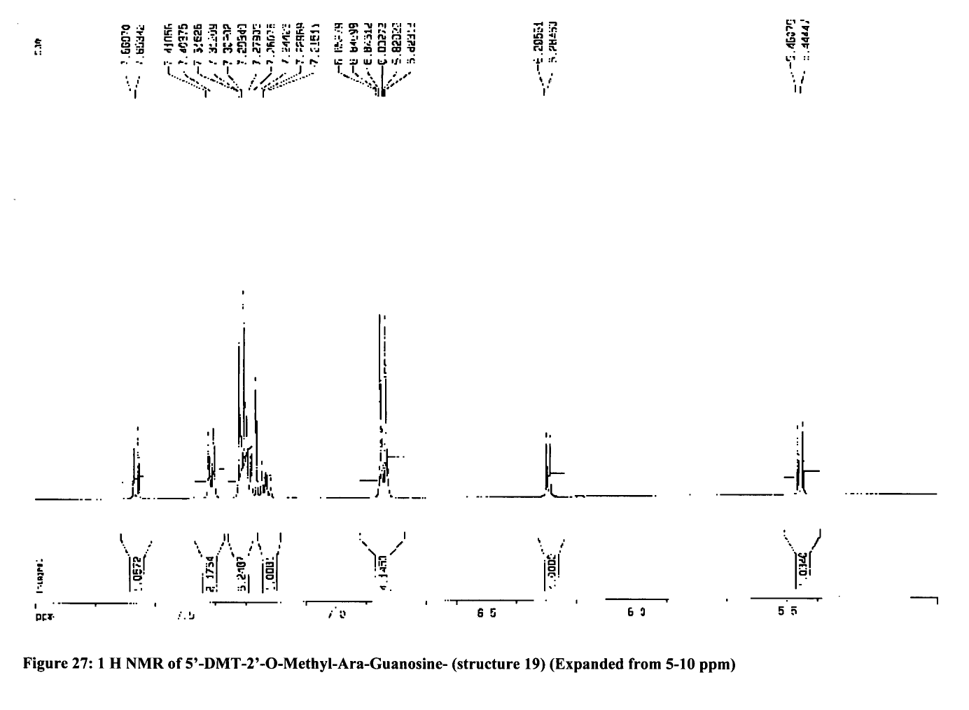

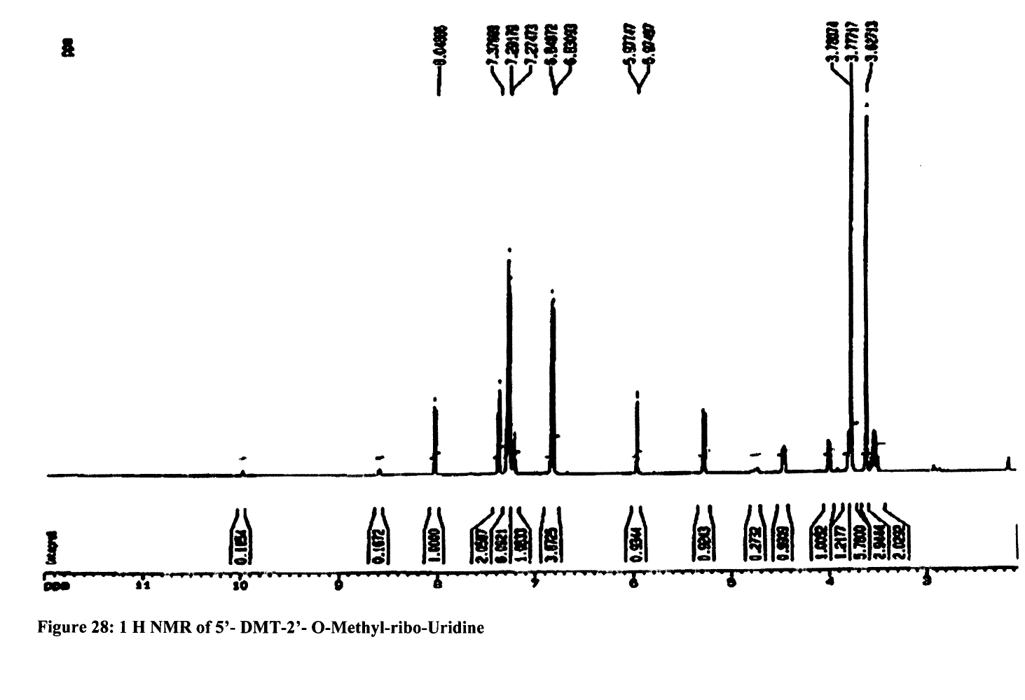

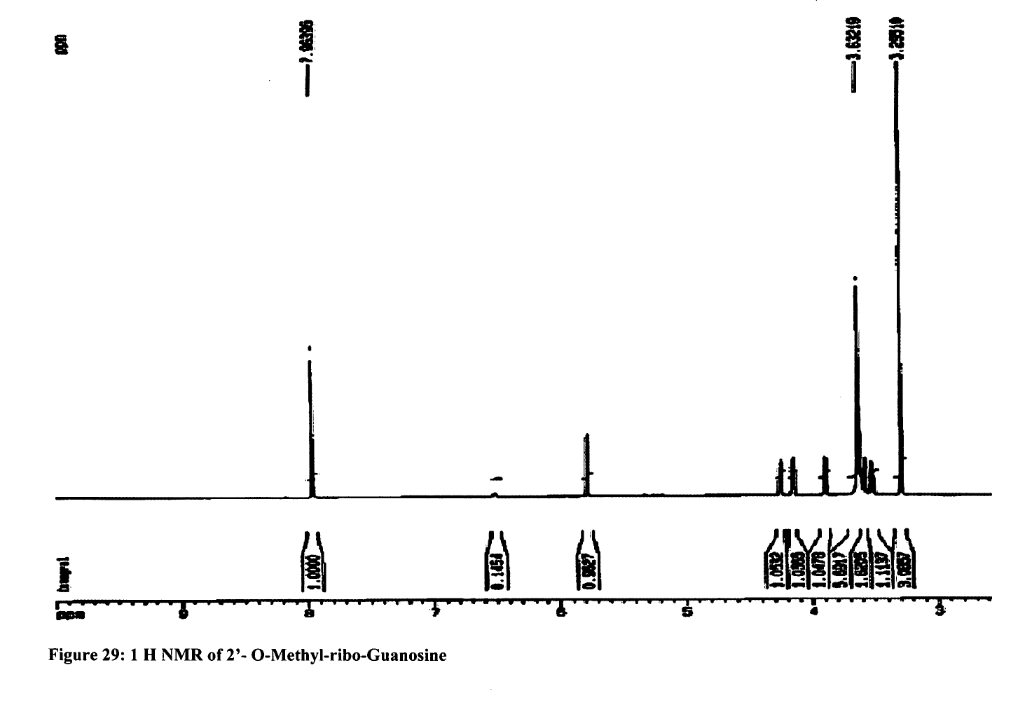

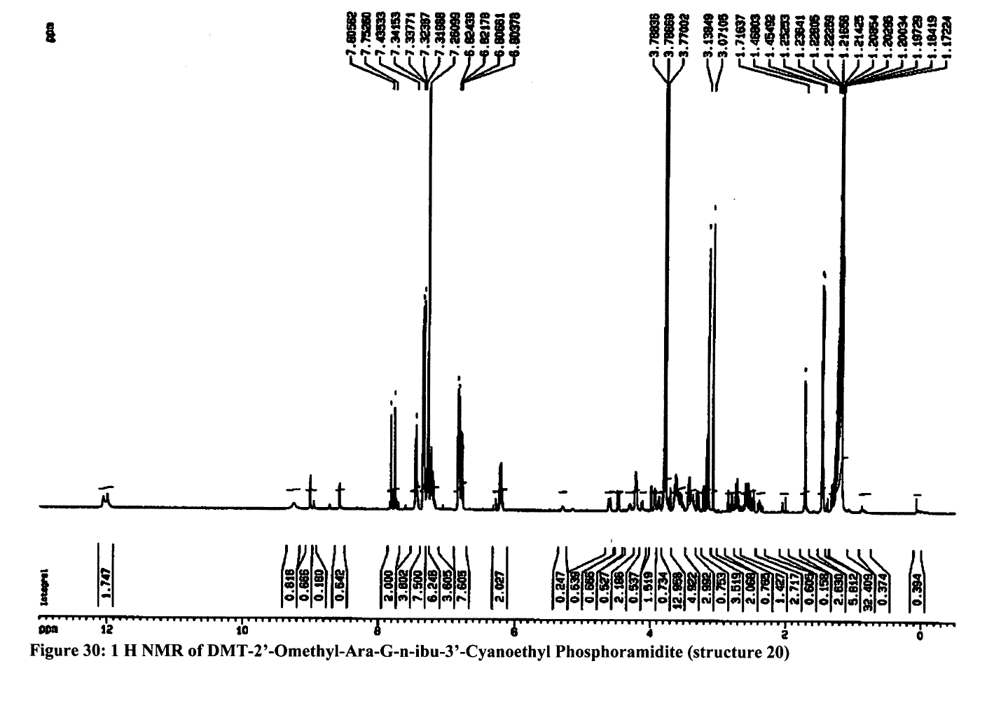

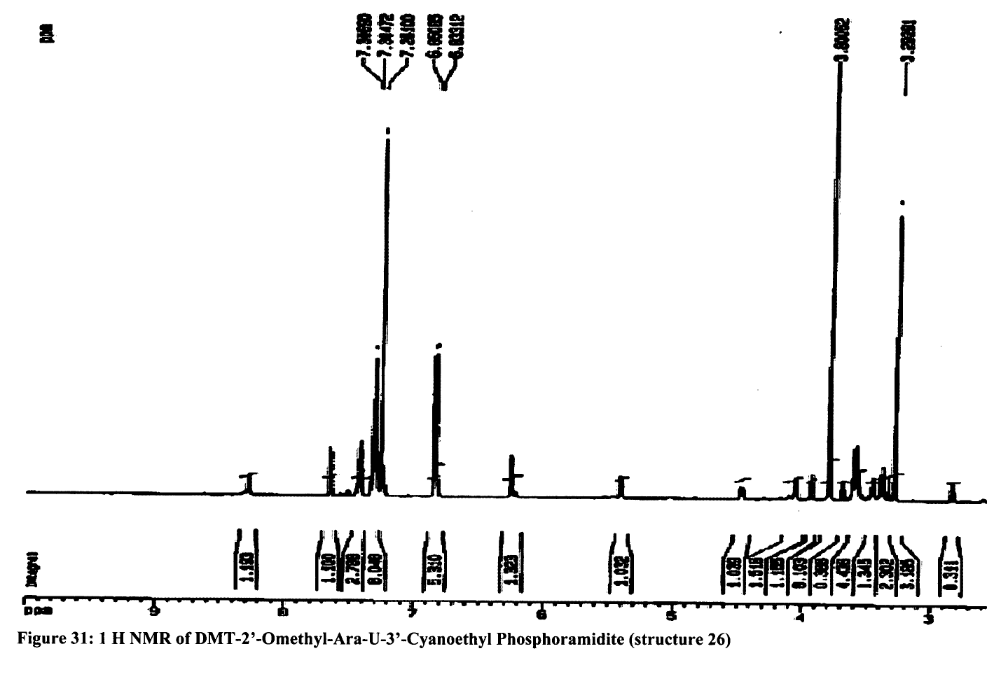

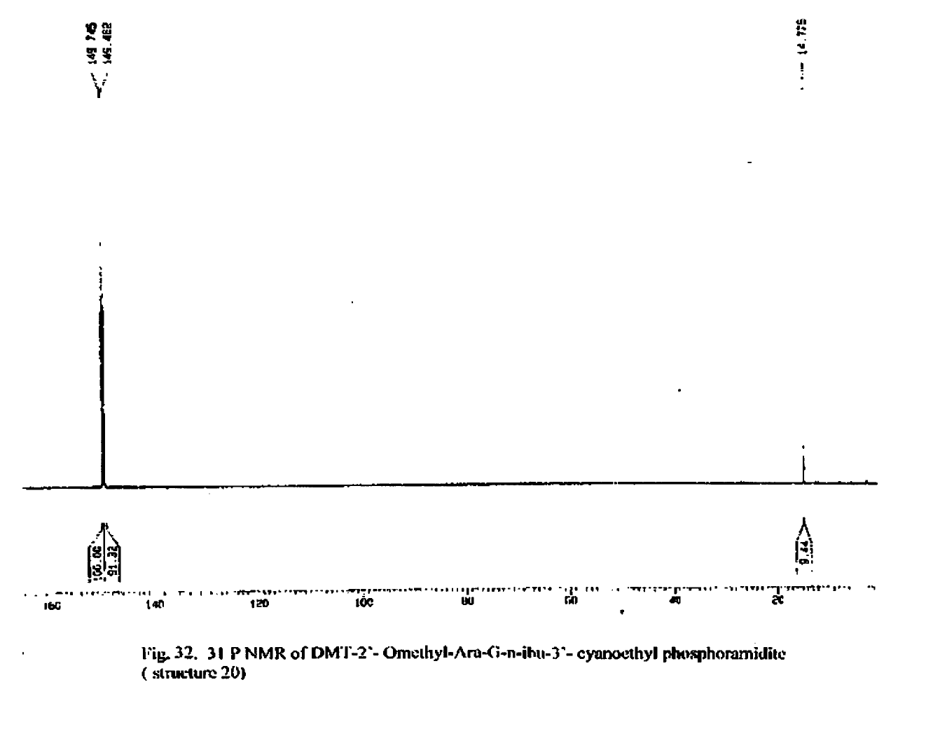

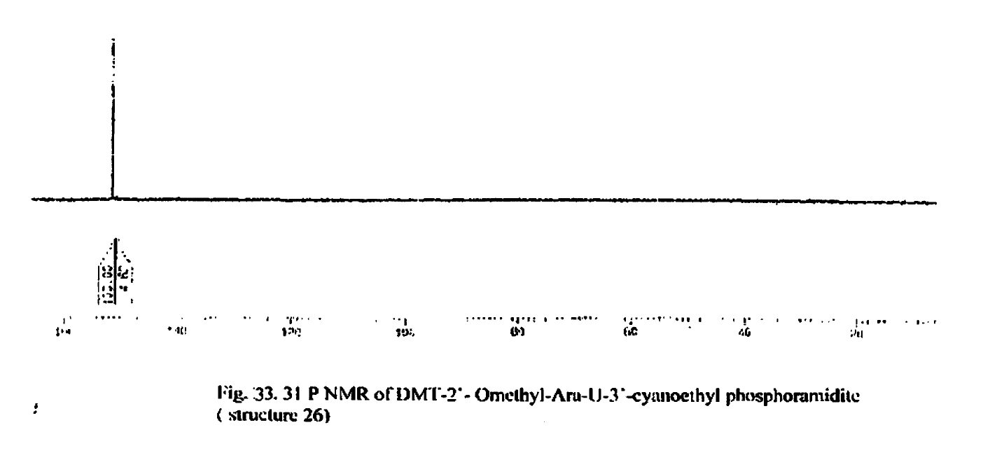

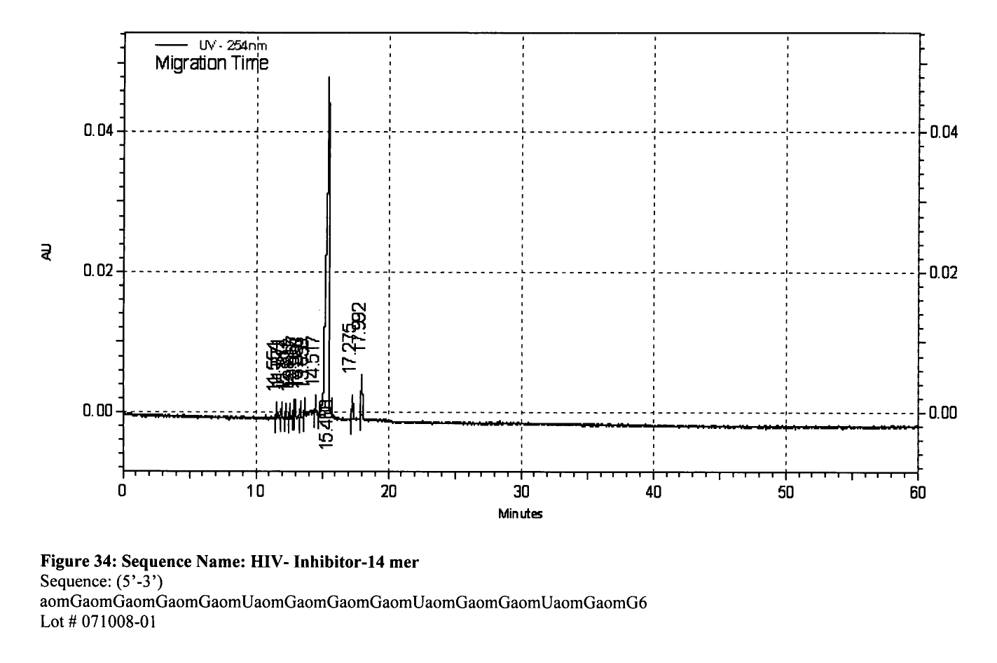

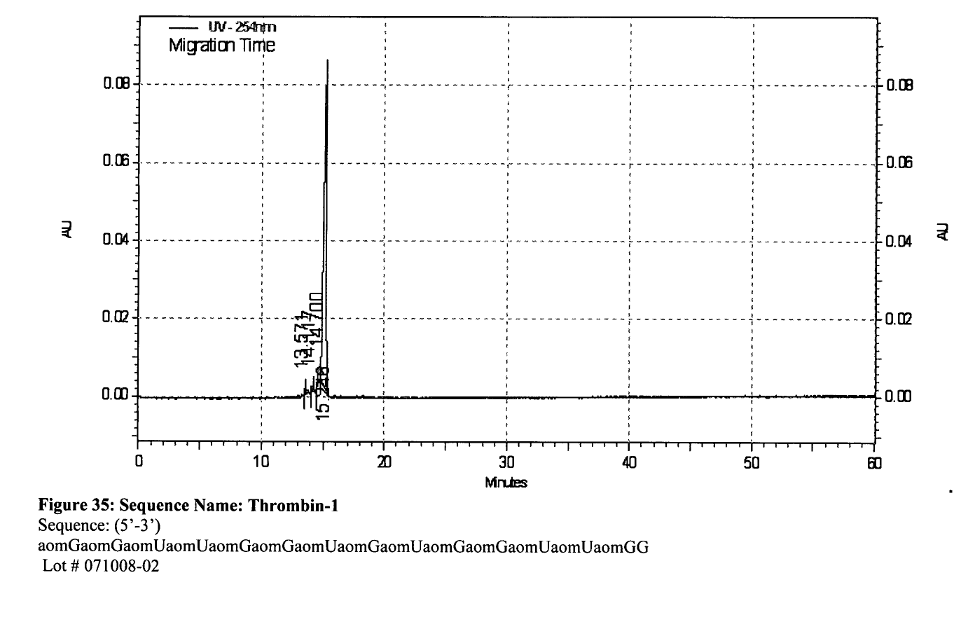

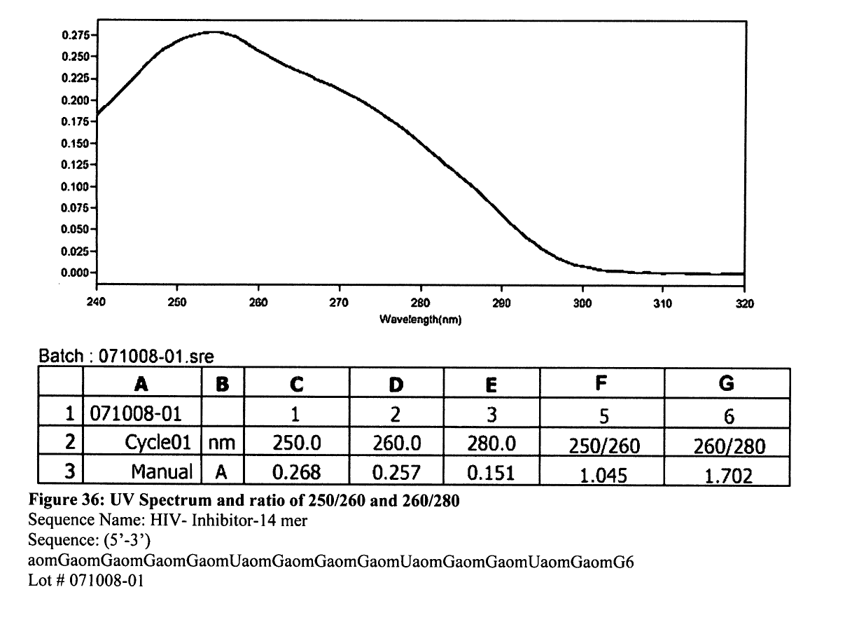

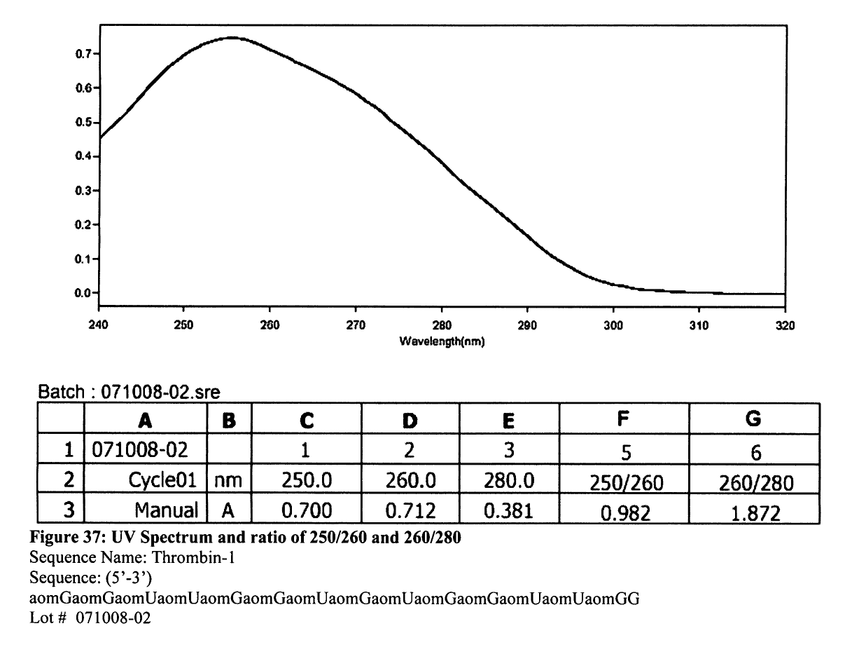

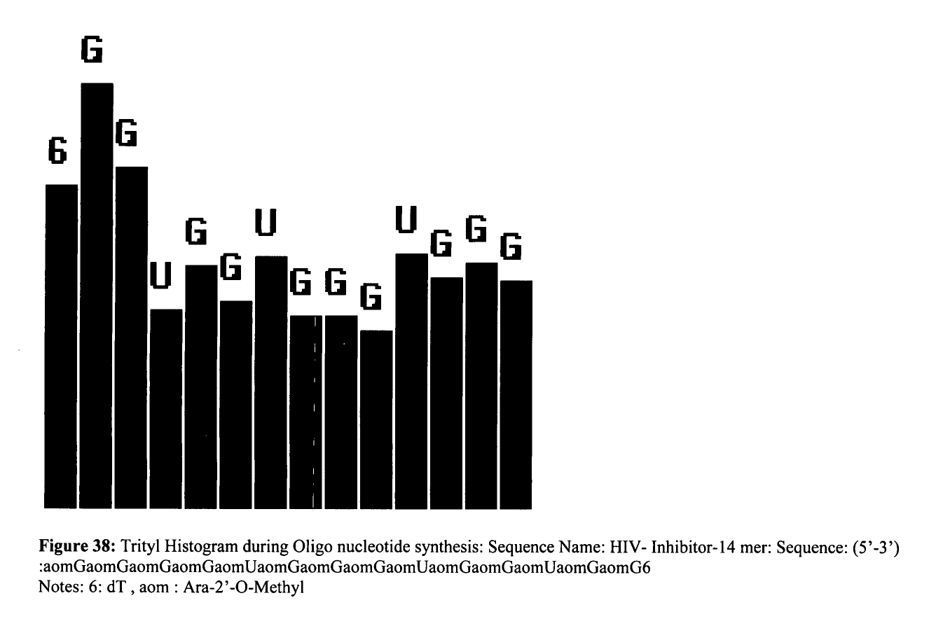

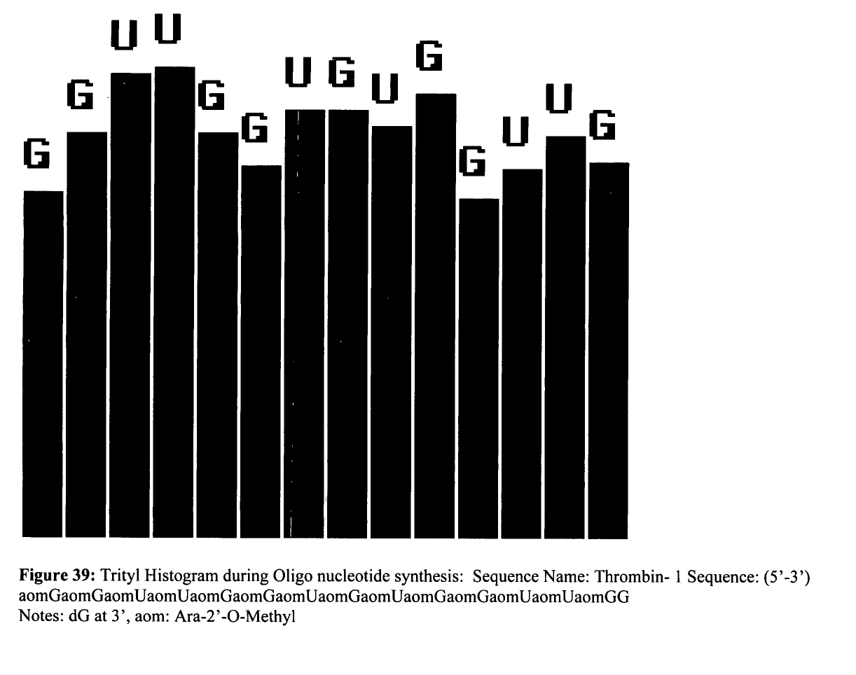

AomGaomGaomUaomUaomGaomGaomUaomGaomUaomGaomGaomUaomUaomGG, where dG at 3′, aom: Ara-2′-O-Methyl;

### SUMMARY OF THE INVENTION

The 2′-Ara-O-Methyl nucleoside and phosphoramidites present opportunity to target various biochemical processes such as antisense, aptamers, and most importantly to develop stable G-quadruplex based oligonucleotides.

The present discovery is based on the development of yet novel nucleoside and the corresponding phosphoramidite molecule and the oligonucleotides derived from them. The 2′-Omethyl-D-arabino nucleic acid analogs, abbreviated as 2′-OMe-ANA, are expected to have improved biochemical and biological properties for targeting DNA and RNA sequences. It is expected that the base would be repelled from the ara-2′-Omethyl substituent, as it is not likely to associate with base protons such as H-6 or H-8 of the pyrimidine and purines respectively.

The concept of introduction of Ara 2′-O-methyl (cis with respect to the nucleoside base) is novel and likely to generate RNA sequences which should possess greater stability with respect to in vivo degradation. Further these molecules are expected to have greater binding capability with the target RNA sequence.

The molecules are expected to behave much like 2′-deoxy nucleosides within DNA/RNA sequences. The introduction of Ara nucleosides as one or more modified bases in the oligo sequences would provide very useful oligonucleotide sequences to study the biochemical role of such modifications.

In the past 2′-0-methyl (trans with respect to nucleoside base and same stereochemistry as natural RNA) modified natural RNA bases have been incorporated into RNA sequences, and such RNA have been developed exclusively for antisense interference of Oligonucleotides, Oligodeoxynucleotides and Oligoribonucleotides. The antisense oligonucleotides containing 2′-O-methyl ribonucleotides in sequence have been shown to cause regulatory interference and lead to therapeutic effects against many diseases and viral infections.

Overwhelming amount of data is available on the selective inhibition of viral protein by the 2′-O-methyl oligonucleotides which are complementary to the portion of m-RNA for specific protein (i.e. antisense). Vast amount of research has been done to lead to potential drug and drug candidate by regulating virus sequences such as HIV many. Such drug candidates have been part of program to lead to ideal antisense based oligonucleotide therapeutic. Several drug candidates are in final phase using 2′-O-Methyl or 2′-O-alkyl modifications.

Therefore there is need to improve and develop modification which could lead to yet better and move effective therapeutic candidate via antisense and RNAi interference, which could lead to better therapeutic agent. They are expected to be stable to the degradation in vivo. Further more 2′-O-methyl Ara oligonucleotide are expected to be efficiently taken up by the cells and would have longer half life within the cell as compared to the natural RNA & DNA sequences. 2′-O-methyl Ara oligonucleotide are expected to effectively interact with the target mRNA sequences.

Besides the potential of 2′-Omethyl-ara-bino nucleoside containing oligonucleosides, the nucleosides of the present research have potential applications in nucleoside bases therapeutics. Thus N-9-[β-D-Arabinofuranosyl]guanine (araG) is a Guanosine nucleoside analog that has shown higher efficiency in T-lymphoblasts compared to B-lymphoblasts.

AraG is relatively resistant to degradation by purine nucleoside phosphorylase (PNP) and the selective cytotoxic effect on T-lymphoblasts is similar to that of deoxyguanosine in the absence of PNP activity. The molecular mechanism mediating this cell specific cytotoxicity of deoxyguanosine and its related analogs is poorly understood. However, a recent study suggests a role of mitochondria in this mechanism with intra-mitochondrial accumulation of dGTP and inhibition of DNA repair. The rate limiting step in araG phosphorylation to its triphosphate form is the initial phosphorylation to its monophosphate form, which is catalyzed by two different enzymes deoxyguanosine kinase (dGK) located in the mitochondrial matrix and deoxycytidine kinase (dCK) located in the cytosol of nucleus. Studies on purified dCK and dGK as well as analysis of araG phosphorylating activities in cell extracts suggest that dGK is the main phosphorylating enzyme of araG at lower concentrations whereas dCK seems to be more important at higher concentrations of araG. These results are consistent with the predominant incorporation of lower concentrations of araG into mtDNA. The dose toxicity in the clinical trials of Nelarabine, of araG, is neurotoxicity. Adverse effects also include myopathy, myelosuppression and the loss of pe sensitivity, similar to the symptoms of drugs mitrochondrial toxicity.

Nucleoside analogs, such as 1-[β-D-arabinofuranosylcytosine, 2-fluoro-2′-arabinofuranosyladenine and 2-chloro-deoxyadenosine, are commonly used in treatment of hematological malignancies. These compounds are transported across the cell membrane by nucleoside transporter proteins and phosphorylated intracellularly to their triphosphate derivatives by nucleoside and nucleotide kinases. The nucleoside analog triphosphates are subsequently incorporated into DNA and cause termination of DNA strand elongation or other DNA lesions. Replication of DNA occurs both in nucleus and in the mitochondrial matrix and there are accordingly two possible targets for nucleoside analogs.

It is anticipated that the oligonucleotides incorporating these monomers will exhibit biological activities related to antisense approach approach, design of better SiRNA's, diagnostic agents. Similarly, it is anticipated that oligonucleotides incorporating such novel nucleosides will be useful to develop therapeutic candidates designing stable G-quadruplexes and Aptamers for oligonucleotide structure, folding topology, evaluation of biochemical properties and design and develop as therapeutic agents.

### DETAILED DESCRIPTION OF THE INVENTION

### Discussion of Synthesis Methodology:

Previously 2′-O-Methyl guanosine derivative has been prepared by monomethylation of a 2′,3′-cis-diol system with diazomethane. The synthesis of N2-isobutyryl-2′-O-methyl guanosine was attempted using methyl iodide and Ag20 on N-1 imino protected N2-isobutyryl 5′,3′-O-TIPDS guanosine and its derivatives. However in each case methylation at base moiety occurred simultaneously²⁸.

Since selective 2′-O-methylation on 5′,3′-TIPDS protected guanosine could not be achieved successfully, methylation on guanosine was carried out on the cis-diol system of 5′-MMT-N2-Ibu-guanosine²⁹ using diazomethane.

There are no procedures known to synthesize protected 2′-O-methyl-arabinonucleosides derivatives. Our procedures are outlined in schemes 1-4, and involve a key step of selective methylation of 5′, 3′- & n-protected ara nucleosides with CH3I and NaH in modest yield.

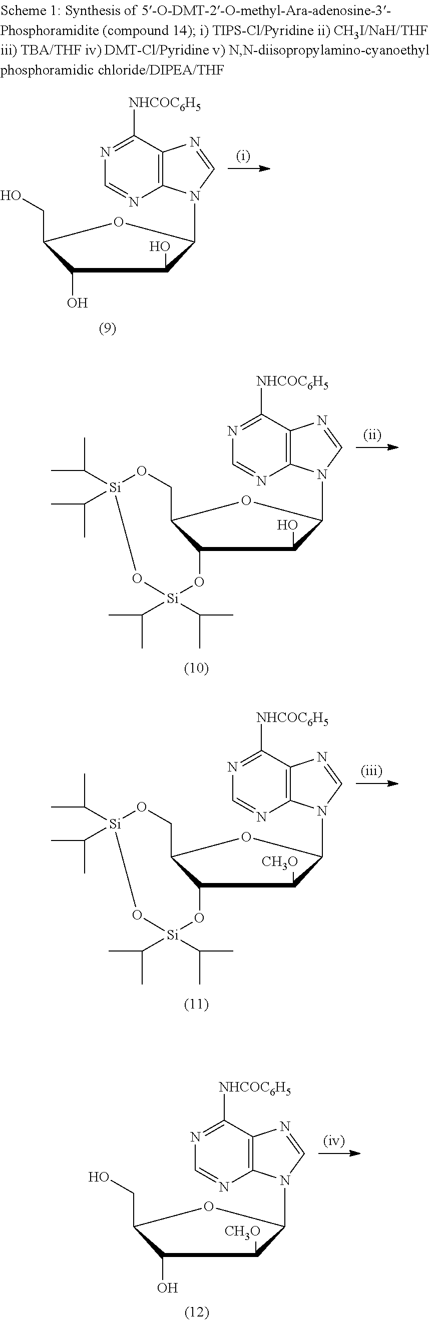

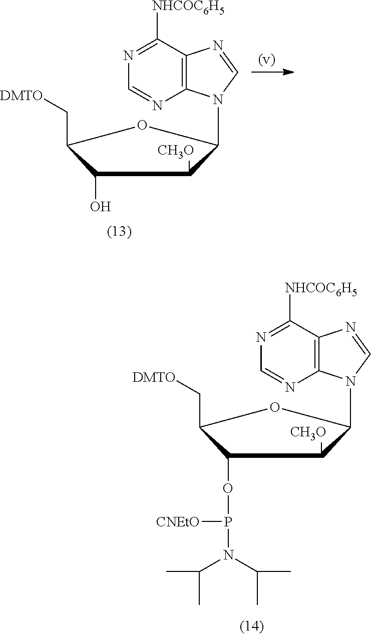

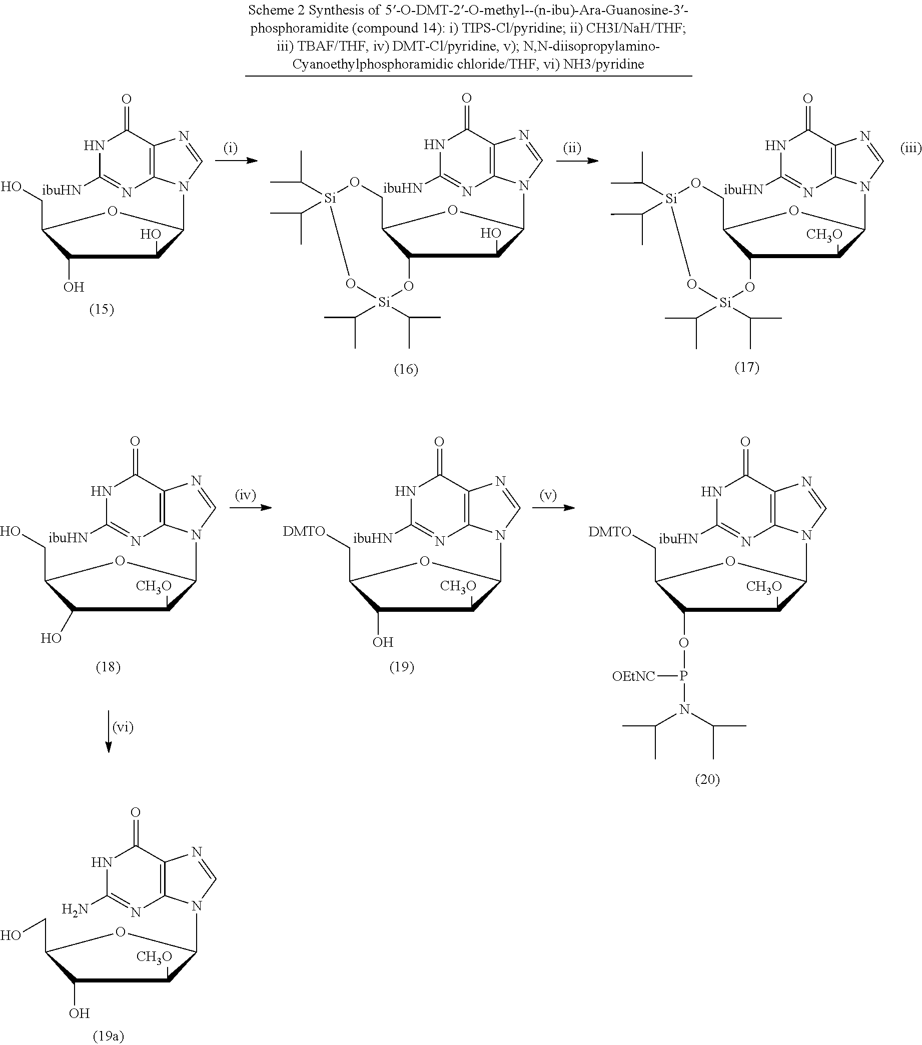

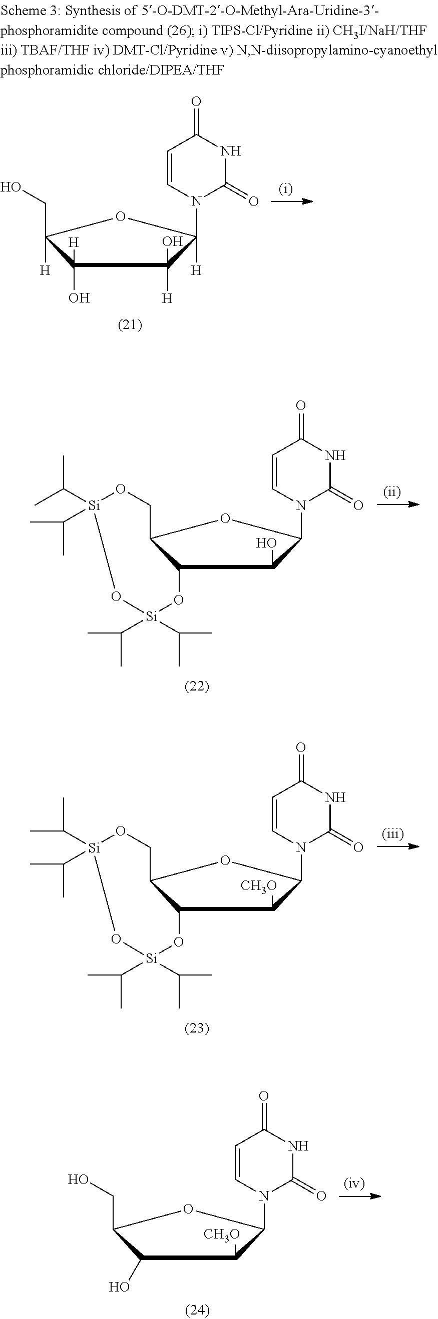

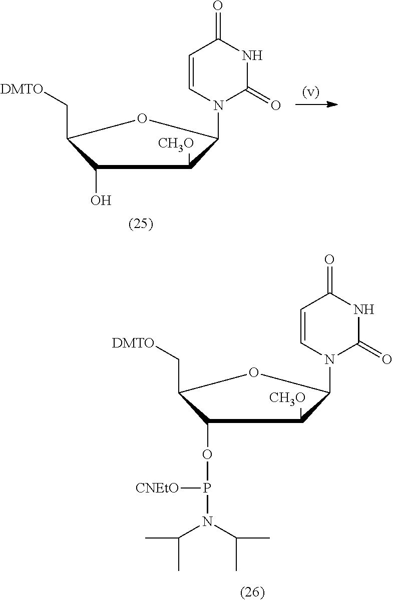

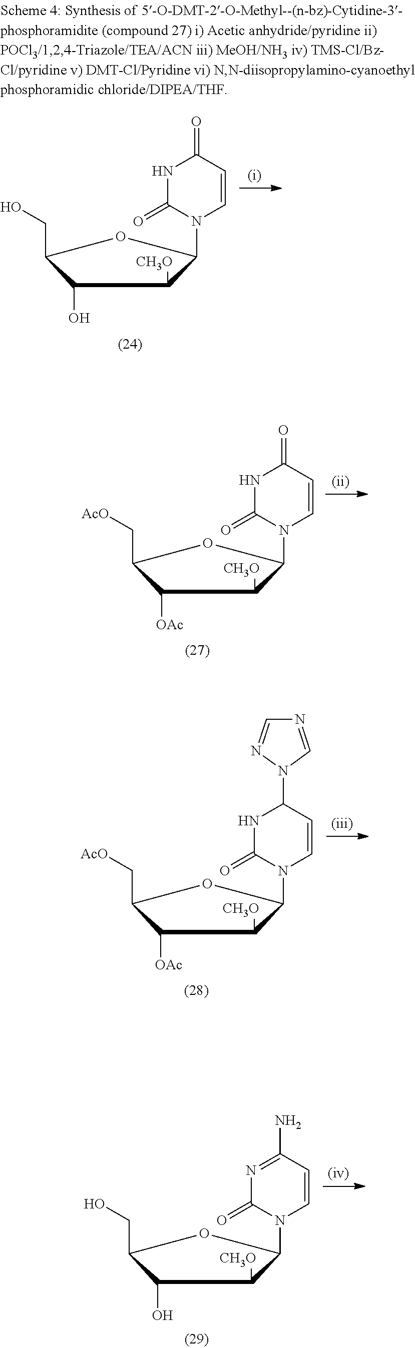

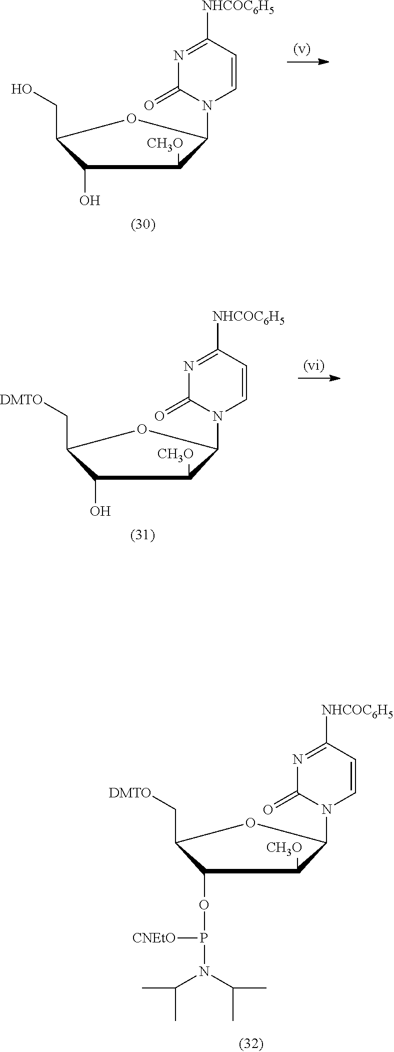

All reactions reported herein were monitored by on TLC plates (Merck silica gel 60 F₂₆₄. The solvent systems were as indicated for individual compounds. UV analysis was carried on Chemito Spectroscan model 2700. The values are reported at 250, 260 and 280 nm and optical density ratio (abbreviated as ORD). HPLC analysis was carried out on Shimadzu instrument, model SCL-10 AVP, and absorbance monitored at 254 and 270 nm wavelengths. The column used was Varian-Microsorb C-18. The proton NMR was carried on 500 MHZ instrument. Mass spectral was analyzed by electro spray ionization, both positive and negative modes.

Abbreviations:

The following abbreviations have been used in the text below reporting experiments: Ac: Acetyl; CAN: Acetonitrile; Bz: Benzoyl; DIPEA: diisopropylethyl amine; DMT: 4, 4′-dimethoxy trityl; Et: ethyl; EtOAc: ethyl acetate; Hex: Hexane; Ibu: isobutryl; Me: methyl; MeOH: Methanol; TIPDS: tetraisopropyldisiloxane; ODR: optical density ratio.

### N⁶-Bz-9-[3,5-O-β-D-arabinofuranosyl]adenine (Compound 9)

It was obtained by Benzoyl Chloride reaction of 2′-Ara-adenosine and followed by partial alkaline hydrolysis.

### N⁶-Bz-9-[3,5-O-(tetraisopropyldisiloxane1,3-diyl)-β-D-arabinofuranosyl]adenine (Compound 10)

1,3-dichloro-1,1,3,3 tetraisopropyldisiloxane (11.4 ml, 34.98 mmol) was added to ice cooled solution of compound 9 (10 gm, 26.93 mmol) in pyridine (120 ml.).

After being stirred at room temperature for 2.5 hrs, methanol (10 ml.) was added to the reaction mixture. The whole mixture was concentrated in vacuum and partitioned between chloroform and saturated bicarbonate solution. The organic layer was washed with water, dried over sodium sulphate and concentrated in vacuum to remove solvents. The residue was subjected to chromatography on a column of silica gel using solvent chloroform:hexane:acetone (50:30:20) with 2% methanol eluting system and pure product was obtained . Yield 8.5 gm, 51.45%. Compound was identified by tlc.

### N⁶-Bz-9-[-(2-O-methyl)-3,5-O-(tetraisopropyldisiloxane-1,3-diyl)-β-D-arabinofuranosyl]adenine (Compound 11)

The sodium salt of compound 10 (7.5 gm, 12.21 mmol) in THF (150 ml.), produced by addition of sodium hydride (0.293 gm, 12.20 mmol) at 10° C. The reaction mixture was stirred for 15 minutes at 10° C., followed by stirring at room temperature for further 15 minutes. To the mixture was added methyl iodide (6.09 ml, 97.73 mmol) drop wise at 20° C. The reaction mixture was tightly sealed and, stirred at 40° C. for three hours. Subsequently, the mixture was concentrated in vacuum and partitioned between chloroform and water dried over sodium sulfate. The organic layer was concentrated in vacuum to remove solvents. The residue was subjected to column chromatography in silica gel using chloroform 2% methanol as gradient as eluting solvent system. Yield 2 gm.

The product of approx 90% purity was obtained and forwarded to next step.

### N⁶-Bz-9-[2-O-methyl-β-D-arabinofuranosyl)]adenine (Compound 12)

Compound (11) (2 gm, 3.18 mmol) was dissolved in THF (20 ml.) and tetrabutyl ammonium fluoride (IM THF solution, 7.96 ml.) was added. The reaction mixture was stirred at room temperature for 1.5 hrs, followed by concentration in vacuum to remove solvents. The crude mixture was charged in a column of silica gel using chloroform with 15% methanol as a gradient elution system. Yield 240 mg.

The product of approx 70% purity was forwarded to next step.

### N⁶-Bz-9-[5-0-4,4′-dimethoxytrityl)-2-0-methyl-β-D-arabinofuranosyl]adenine (Compound 13)

Compound 12 (240 mg,0.62 mmol) was dissolved in 2.88 ml. pyridine and the reaction mixture was cooled up to 0° C. DMT-Cl (25 mg, 0.74 mmol) was added in five portions at time intervals of 30 minutes. The reaction mixture was stirred at 0° C. for an additional one hour. The TLC was checked in chloroform with 5% methanol. The reaction mixture was quenched with methanol and subsequently two third pyridine was concentrated in vacuum and partitioned between chloroform and saturated aqueous bicarbonate solution. The organic layer was washed with water, followed by drying over anhydrous sodium sulphate. The solution was subsequently concentrated in vacuum to remove solvents. The residue was subjected to column chromatography on silica gel using a gradient solvent system of chloroform:hexane:acetone (50:30:20) with 5% methanol as for elution and pure product was obtained as foam. Yield 70 mg, 16.35%. Compound was identified by tlc, uv spectral analysis and HPLC.

### N⁶-Bz-9-[5-0-4,4′-dimethoxytrityl)-2-0-methyl-3-O-{bis(1-methylethyl)amino-(2-cyanoethoxy)phosphinyl}-β-D-arabinofuranosyl]adenine (compound 14)

### N²-Ibu-9-[β-D-arabinofuranosyl]guanine (Compound 15)

It was obtained by isobutyrylation of Ara G and followed by partially alkaline hydrolysis.

### N²-Ibu-9-[3,5-O-(tetraisopropyldisiloxane-1,3-diyl)-β-D-arabinofuranosyl]guanine (Compound 16)

1,3-Dicholoro-1,1,3,3 tetraisopropyldisiloxane (6.9 ml, 21.68 mmol) was added to ice cooled solution of compound 15 (3.0 gm, 8.49 mmol) in pyridine (36.9 ml). After being stirred at room temperature for 3.0 hours, methanol (3 ml) was added to the reaction mixture. The whole mixture was concentrated under vacuum and partitioned between chloroform and saturated bicarbonate solution. The organic layer was washed with water, dried over anhydrous sodium sulphate and concentrated under vacuum to remove solvents. The residue was subjected to chromatography on a column of silica gel using 3% methanol as eluting solvent system. The pure fractions were concentrated to a yield a foam (44.17%). Compound was analyzed by tlc and uv spectrum.

### N²-Ibu-9-[2-O-methyl-(3,5-O-(tetraisopropyldisiloxane-1,3-diyl)-β-D-arabinofuranosyl]guanine (Compound 17)

To the compound 16 (2.2 gm, 3.69 mmole) in THF (distilled over LiAH4; 44 ml) was added sodium hydride (0.088 gm, 3.6 mmol) at 10 C under anhydrous conditions. The reaction mixture was stirred for 15 min at 10 C followed by stirring at room temperature for further 15 min. To the mixture was added methyl iodide (2.3 ml, 36.91 mmol), drop wise at 25 C. The reaction mixture was tightly sealed and stirred at 40° C. for three hours. Subsequently the mixture was concentrated under vacuum and partitioned between chloroform and water, followed by drying over anh sodium sulphate. The organic layer was concentrated under vacuum to remove solvents. The residue was purified by column chromatography on silica gel (70-230 mesh particle size A) using chloroform containing 2% methanol as a gradient elution system. The pure fractions were concentrated as a foam (500 mg, 23.0% yield). Compound was analyzed by tlc and uv.

### N²-Ibu-9-[2-0-methyl-β-D-arabinofuranosyl]guanine (Compound 18)

Compound 17 (500 mg, 0.82 mmole) was dissolved in THF (5 ml) and tetrabutyl ammonium fluoride (1M THF solution, 2.05 ml) was added. The reaction mixture was stirred at room temperature for 1.5 hours, followed by concentration in vacuum to remove solvents. The crude reaction mixture was charged in a column of silica gel (70-230 mesh particle size A°) using chloroform with 15% methanol as a gradient elution system; pure product was obtained (250 mg, 82.78% yield) as a crystalline solid. HPLC Analysis: purity 98.5%.

### N²-Ibu-9-[5-O-(4,4′-dimethoxytrityl)-2-O-methyl-β-D-arabinofuranosyl]guanine (Compound 19)

Compound 18 (250 mg, 0.68 mmole) was dissolved in pyridine (3 ml) and the reaction mixture was cooled up to 0° C. DMT-Cl (1.2 eq, 0.276 gm) was added in five portions at a time interval of 30 minutes and the reaction mixture was stirred at same temperature for an additional one hour. The reaction was quenched with methanol. ⅔rd pyridine was concentrated under vacuum and partitioned between chloroform and saturated sodium bicarbonate solution. The organic layer was washed with water, followed by drying over anhydrous sodium sulfate, concentrated under vacuum, to remove solvents. The residue was subjected to column chromatography on silica gel (70-230 mesh particle size A°) using a gradient system of chloroform:hexane:acetone (5.0:3.0:2.0) with 5% methanol as an eluant. Pure compound (300 mg, 65.93% yield) was obtained as a foam. HPLC Analysis: purity 99.06%. Mass Spectral Analysis; calculated; 671.85, observed ; m/e; 670.8.

### 9-[2-O-methyl-β-D-arabinofuranosyl]guanine (Compound-19a)

Compound 18 (0.1 gm, 0.27 mmol) was dissolved in pyridine (1.0 ml), followed by addition of 25% methanolic ammonia solution (1.0 ml). The reaction mixture was sealed and left for 24 hours at 40° C. Total solvent was evaporated and co-evaporated twice with 5.0 ml portion of acetonitrile under vacuum. The residue was washed thrice with 10 ml portion of diethyl ether and the solvent was decanted. The gummy mass was crystallized with minimum ethanol. The product was filtered and dried. Yield; 28.0 mg, 35.0%. HPLC Analysis; purity 99.23%; Mass Spectral Analysis; calculated; 297.23, observed; m/e; 320.30(M+23).

### N²-Ibu-9-[5′-O-(4,4′-dimethoxytrityl)-2-O-methyl-3-O-{bis(1-methylethyl)amino-(2-cyano ethoxy)phosphinyl}-β-D-arabinofuranosyl]guanine (Compound 20)

Compound 19 (0.9 gm, 1.3 mmol) was dissolved in THF (7.0 m1) and added DIPEA (0.93 ml, 5.55 mmol). The reaction mixture was cooled up to 0° C. and n,n-diisopropylamino-cyanoethyl-phosphoramidic-chloride (0.59 ml, 264 mmol) was added drop wise followed by stirring for one and half hour at room temp. The reaction mixture was partitioned between ethyl acetate and saturated aqueous sodium bicarbonate solution. The organic layer was washed with brine solution and dried over sodium sulfate.

The residue was subjected to chromatography on a column of silica gel and eluting solvent system chloroform:ethyl acetate:tri ethyl amine (5.0:4.0:1.0) and ethyl acetate:acetone triethylamine (6.0:3.0:1.0) as gradient. The pure fractions were concentrated to a foam. The compound was precipitated using solvent system hexane:ethyl acetate (8.5:1.5) after dissolving in minimum chloroform, followed by decanting the solvent. Finally taking the gummy mass in acetonitrile and filtering the solid and drying. Yield; 700 mg, 60%, tlc; ethyl acetate:hexane:tri ethyl amine (6.0:3.0:1.0) R_{f} value—(see table), UV Spectrum; λₘₐₓ; Emax=, see table.

HPLC Analysis:

### 1-[3,5-O-(tetraIsopropyldisiloxane-1,3-diyl)-β-D-arabinofuranosyl)]uracil (Compound 22)

Compound 21 (10.0 gm., 41.28 mmol) was suspended in dry pyridine (150 ml.) and stirred. 1,3-dichloro-1,1,3,3-tetraIsopropyl-disiloxane (17.34 ml, 53.51 mmol) was added drop wise at 0° C. After addition of reagent the reaction mixture was left on stirring for 3.0 hrs at room temperature. Subsequently two third pyridine was evaporated on rota evaporator and the residue taken in 200 ml. chloroform and treated with saturated aq. sodium bicarbonate solution (150 ml) and organic layer was separated and aqueous layer was extracted twice with 75 ml. portion of chloroform. Organic layer was passed through anhydrous sodium sulfate and evaporated on rotavapor.

The crude product was purified on silica gel column using the solvent mixture of chloroform hexane:acetone (50:30:20) containing 1% and 2% methanol Yield 12 gm., 60.3%. Compound was analyzed by tlc and uv spectrum.

### 1-[2-O-methyl-3, 5-O-(tetraIsopropyldisiloxane-1,3diyl)-β-D-arabinofuranosyl]uracil (Compound 23)

The sodium salt of compound 22 (18 gm., 37.15 mmol) is prepared in THF by addition of sodium hydride (1.78 gm., 74.16 mmol) at 10° C. The mixture was stirred at 10° C. for 15 minute. To the mixture was added methyl Iodide (6.94 ml, 111.38 mmol) at room temperature in four portions in 30 minute interval. After the addition, the reaction mixture was stirred at 10° C. for additional 2 hrs. Subsequently, the mixture was concentrated in vacuum and partitioned between chloroform and water, dried over sodium sulphate. The organic layer was concentrated under vacuum to remove solvents. The crude product was purified by chromatography on silica gel column using solvent system chloroform: hexane: acetone (50:30:20). The pure fraction was concentrated as a foam. Yield; 18.5 gm, 62.0%. Compound was identified by tlc and uv spectrum.

### 1-[2-O-methyl-β-D-arabinofuranosyl]uracil (Compound 24)

Compound 23 (12 gm, 24 mmol) was dissolved in 120 ml THF and 60 ml tetra butyl ammonium fluoride (1M THF solution) was added. The reaction mixture was stirred at room temperature for 1.5 hrs, followed by concentration under vacuum to remove solvents. The crude mixture was charged in a column of silica gel using chloroform with 15% methanol as a gradient elution system. Yield 6.0 gm., 97.4% Compound was identified by tlc and uv spectrum. HPLC analysis: purity 100%; Mass spectral analysis; calculated; 258.23, observed; 281.3(M+23)

### 1-[5-O-(4,4′-dimethoxytrityl)-2-O-methyl-β-D-arabinofuranosyl]uracil (Compound 25)

Compound 24 (720 mg., 2.81 mmol) was dissolved in pyridine (8.6 ml.) and the reaction mixture was cooled up to 0° C. DMT-Cl (1.14 gm., 3.36 m mol) was added in live portions at time interval of 30 minutes and the reaction mixture was stirred at same temperature for additional one hour. The tlc was checked in chloroform with 5% methanol. The reaction was quenched with methanol followed by removal of approximately two third pyridine on rotary evaporator. The residue was taken in chloroform and washed with saturated aqueous sodium bicarbonate solution. The organic layer was washed with water followed by drying over anhydrous sodium sulfate, the filtrate was concentrated in vacuum to remove solvents. The crude product was purified on silica gel using a gradient system of chloroform:hexane:acetone (50:30:20) with 5% methanol as an eluant. Yield; 1.1 gm, 70.5%. HPLC analysis; purity 99.68%; Mass spectral analysis; calculated; 560.56, observed; 583.6; dimer; calculated mass; 1121.12, observed; 1144.5(dimer+Na).

### 1-[5-O-4, 4′-dimethoxytrityl-2-O-methyl-3-O-{bis(1-methylethyl)amino-(2-cyano ethoxy)phosphinyl}-β-D-arabinofuranosyl]uracil (Compound 26)

Compound 25 (0.6 gm, 1.07 mmol) was dissolved in THF (5 ml) and added DIPEA (0.74 ml, 4.27 mmol). The reaction mixture was cooled up to 0° C. and N,N-Diisopropylamino cyanoethyl phosphoramidic chloride (0.47 ml, 2.14 mmol) was added drop wise followed by stirring for one and half hour at room temp. The reaction mixture was partitioned between ethyl acetate and saturated aqueous sodium bicarbonate solution. The organic layer was washed with brine solution, dried over sodium sulfate and concentrated in vacuum to remove solvents. The crude was purified by column chromatography with silica gel using EtoAc:hexane:TEA (5.0:4.0:1.0) as running and eluting solvent system. The pure fractions were concentrated to a foam. The foam was dissolved in Acetonitrile, filtered and redried under vaccume. Yield; 300 mg, 40%. Tlc; Etoac:hexane:TEA (5.0:4.0:1.0), Rf value; uv spectrum are recorded in tables. HPLC Analysis).

### 1-[3, 5-di-O-acetyl-2-O-methyl-β-D-arabinofuranosyl]uracil (Compound 27)

Compound 24 (6 gm 23.23 mmol) was dissolved in (72.0 ml.) pyridine and acetic anhydride (10.96 ml, 105.98 m mol) was added the reaction mixture was stirred at room temperature for 3 hrs followed by concentrated in vacuum to remove two third of pyridine. The residue was taken in chloroform and washed with saturated aqueous sodium bicarbonate solution. The organic layer was subsequently followed by drying over sodium sulfate, concentrated in vacuum, to remove solvents. The crude mixture was taken in hexane:ethyl acetate (70:30) 150 ml and shacked well. Solid was filtered and washed with hexane ethyl acetate (70:30) mixture and this is resulted pure product. Yield 6.5 gm, 82.2%. Compound was identified by tic and uv spectrum recorded in tables.

### 4-triazolyl-1-[3,5-di-O-acetyl-2-O-methyl-β-D-arabinofuranosyl]uracil Compound (28)

1,2,4 triazole (20.33 gm., 294.33 mmol) was suspended in anhydrous acetonitrile (65.0 ml.) and phosphoryl chloride (5.27 ml., 56.60 mmol) was added drop wise at 0° C. with stirring followed by addition of triethylamine 41.26 ml., 294.36 mmol after addition of triethylamine the reaction mixture was diluted with anhydrous acetonitrile (65 ml). This mixture was added to a solution of the compound 27 (6.5 gm, 18.87 mmol) in anhydrous acetonitrile (65 ml.) at 0° C. under nitrogen atmosphere. The reaction mixture was stirred for additional 2 hrs at room temperature. Subsequently two third volume of the solvent was evaporated and compound was dissolved in chloroform (100 ml.) and washed with water. The organic layer was dried over anhydrous sodium sulphate and concentrated in vacuum to remove the solvent.

The crude reaction mixture was charged in column of silica gel using chloroform with 30% acetone and pure product was obtained as foam to yield 6.5 gm, 86.0%. Compound was identified by tlc and uv spectrum.

### 1-[2-O-methyl-β-D-arabinofuranosyl]cytosine (Compound 29)

Compound 28 (6.5 gm., 16.39 mmol) was taken in 20% methanolic ammonia (65 ml.). The reaction mixture was tightly sealed and left for 30 hrs at 30° C. solvent was evaporated and co evaporated twice with 20 ml. acetonitrile. The crude reaction mixture was charged in column of silica gel using chloroform with 25% methanol as a gradient elution system and pure product was obtained to yield 6.5 gm, 70.0%. HPLC analysis: purity 99.94%.

Mass spectral analysis; calculated; 257.24, observed; 258.1& 280 (M+23). dimer present;calculated;(514.48),observed;515.6& 537.5(dimer+Na).

### N⁴-Bz-1-[2-o-methyl-β-D-arabinofuranosyl]cytosine (Compound 30)

Compound 29 (3 gm., 11.74 mmol) was suspended in dry pyridine (36.0 ml.) and trimethylchlorosilane (4.43 ml., 34.97 mmol) was added drop wise at 0° C. The reaction mixture was stirred for one & half hour at room temperature. The reaction mixture was cooled up to 0° C. and benzoyl chloride (2.71 ml, 23.32 mmol) was added drop wise, followed by stirring for 3 hrs at room temperature. Reaction mixture was cooled up to 0° C. and added cold distilled water (10 ml.) at same temperature after 15 minute cold aqueous ammonia solution 10 ml. was added and the reaction mixture was stirred for further 20 minute at the same temperature. The reaction mixture was concentrated under vacuum up to dryness. The crude mixture was taken in 25 ml. water and solid was filtered and washed with (10 ml.) water, followed by washed with ethyl acetate and diethyl ether respectively and pure product was obtained as crystalline solid. Yield; 2.66 gm, 63.33%. HPLC analysis; purity 99.34%

### N⁴-Bz-1-[5-O-4, 4′-dimethoxytrityl-2-O-methyl-β-D-arabinofuranosyl]cytosine (Compound 31)

Compound (30) (1.66 gm., 4.59 mmol) was dissolved in dry pyridine (19.92 ml.) and the reaction mixture was cooled up to 0° C. DMT-Cl (1.86 gm, 5.48 m mol) was added in five portions at time intervals of 30 minutes. The reaction mixture was stirred at 0° C. for an additional one hour. The tlc was checked in chloroform with 5% Methanol. The reaction mixture was quenched with methanol, and subsequently two third pyridine was concentrated under vacuum and partitioned between chloroform and saturated aqueous bicarbonate solution. The organic layer was washed with water followed by drying over anhydrous sodium sulfate. The solution was subsequently concentrated under vacuum to remove solvents. The residue was subjected to column chromatography on silica gel using a gradient solvent system of chloroform:hexane:acetone (50:30:20) with 6% methanol as an elution and pure product was obtained as a foam. Yield; 2.81 gm, 77.0%; Mass Spectral Analysis; calculated; 663.57, observed: 664.7&686.7(M+23), dimer present; calculated; 1327, observed; 1350.4(dimer+Na).

### N⁴-Bz-1-[5-O-4, 4′-dimethoxytrityl-2-O-methyl-3-O-{bis(1-methylethyl)amino-(2-cyano ethoxy)phosphinyl}-methyl-β-D-arabinofuranosyl]cytosine (Compound 32)

TABLE A
Tlc analysis
(2′-O—Me--Ara-Nucleosides)

| Compound | | | |
|---|---|---|---|
| # | Nucleoside | Solvent System | R_{f} value |
| 1. | | | |
| 29 | C | Chl:MeoH (8.0:2.0) | 0.35 |
| 19a | G | Chl:MeoH (7.5:2.5) | 0.20 |
| 24 | U | Chl:MeoH (8.5:1.5) | 0.30 |

TABLE B
Tlc analysis
(2′-O—Me—N protected-Ara-Nucleosides)

| Compound | Protected | | |
|---|---|---|---|
| # | Nucleoside | Solvent System | R_{f} value |
| 12 | n-bz-A | Chl:MeoH (9.0:1.0) | 3.0 |
| 30 | n-bz-C | Chl:MeoH (9.0:1.0) | 0.26 |
| 18 | n-ibu-G | Chl:MeoH (8.5:1.5) | 0.2 |

TABLE C
Tlc analysis
(5′-DMT-2′-O—Me—N protected-Ara-Nucleosides)

| Compound | Protected | | |
|---|---|---|---|
| # | Nucleoside. | Solvent System | R_{f} value |
| 13 | n-bz-A | chl:hex:acetone(5.0:3.0:2.0)with | 0.6 |
| | | MeoH 5% | |
| 31 | n-bz-C | chl:hex:acetone(5.0:3.0:2.0)with | 0.5 |
| | | MeoH 6% | |
| 19 | n-ibu-G | chl:hex:acetone(5.0:3.0:2.0)& with | 0.4 |
| | | MeoH 5% | |
| 25 | U | chl:hex:acetone(5.0:3.0:2.0)with | 0.5 |
| | | MeoH 5% | |

TABLE D
UV analysis

| | | | Abs. | Abs. | | | |
|---|---|---|---|---|---|---|---|
| | | | at | at | Ratio | Ratio | |
| Comp | Protected | Abs at | 260 | 280 | 250/ | 260/ | Emax at |
| No. # | Nucleoside. | 250 nm | nm | nm | 260 | 280 | λ.ₘₐₓ |

(5′-DMT-2'-O—Me—N protected-Ara-Nucleosides)

| | | | | | | | |
|---|---|---|---|---|---|---|---|
| #13 | n-bz-A | 0.652 | 0.538 | 0.769 | 1.21 | 0.69 | 21920 |
| | | | | | | | at 280 nm |
| #31 | n-bz-C | 0.770 | 0.792 | 0.377 | 0.97 | 2.10 | 18820 |
| | | | | | | | at 260 nm |
| #19 | n-ibu-G | 0.488 | 0.445 | 0.348 | 1.09 | 1.27 | 19805 |
| | | | | | | | at 250 nm |
| #26 | U | 0.577 | 0.550 | 0.318 | 1.05 | 1.73 | 11552.3 |
| | | | | | | | at 250 nm |

2′-O—Me—N protected-Ara-Nucleosides

| | | | | | | | |
|---|---|---|---|---|---|---|---|
| #12. | n-bz-A | 0.495 | 0.524 | 0.764 | 0.94 | 0.68 | 16662.5 |
| | | | | | | | at 260 nm |
| #30 | n-bz-C | 0.893 | 1.047 | 0.427 | 0.85 | 2.46 | 13498 |
| | | | | | | | at 260 nm |
| #15 | n-ibu-G | 0.90 | 1.02 | 0.76 | 0.92 | 1.34 | 14354.9 |
| | | | | | | | at 260 nm |

TABLE E
Proton NMR of 2′-OMe (ara) nucleosides:

| | 2′- | | | | | | | | | H-5′ & |
|---|---|---|---|---|---|---|---|---|---|---|
| Ncleoside | OCH₃ | H2 | H8 | H5 | H6 | H-1′ | H-2′ | H-3′ | H-4′ | H-5″ |
| A | | | | | | | | | | |
| C | 3.15 | NA | NA | 5.69 & | 7.55 & | 6.12-6.13; d | 3.96-3.98; t | 3.74-3.76; | 3.65-3.68; | 3.58-3.52; m |
| | | | | 5.71; d | 7.56; d | | | qt | qt | |
| G | 3.14 | NA | 7.75; s | NA | NA | 6.10-6.11; m | 4.18-4.21; t | 3.90-3.91; t | 3.68-3.91; | 3.56-3.63; m |
| | | | | | | | | | qt | |
| U | 3.24 | NA | NA | 5.58 & | 7.63 & | 6.10-6.12; m | 4.00-4.02; t | 3.83-3.85; | 3.65-3.68; m | 3.59-3.62 & |
| | | | | 5.59; d | 7.64; d | | | qt | | 3.52-3.54; m |

TABLE F
Proton NMR of 5′-DMT-2′-OMe (Ara)-N-Protected) nucleosides:

| | 2′- | | | | | | | | | | H-5′ & | CH | CH₃ |
|---|---|---|---|---|---|---|---|---|---|---|---|---|---|
| Nucle- | ara- | DMT | | | | | | | | | H- | of | of |
| oside | OCH₃ | (O—CH₃)₂ | H2 | H8 | H5 | H6 | H-1′ | H-2′ | H-3′ | H-4′ | 5″ | ibu | ibu |
| (n- | | | | | | | | | | | | | |
| bz)A | | | | | | | | | | | | | |
| (n- | 3.22 | 3.79 & | NA | 7.75 | 7.57 & | 8.02 & | 6.42-6.43; | 4.26-4.28; | 4.13-4.14; | 4.10-4.11; | 3.37-3.43; | NA | NA |
| bz)C | | 3.80 | | | 7.58; d | 8.03; d | d | t | m | m | m | | |
| (n- | 3.03 | 3.70 | NA | 7.81 | NA | NA | 6.09 & | 4.46; | 4.17-4.18; | 3.76; m | 3.44-3.29; | 2.82-2.86; | 1.16-1.25; |
| ibu)G | | | | | | | 6.10 (d) | br | t | | m | qt | qt |
| U | 3.33 | 3.78 | NA | NA | 5.44 & | 7.65 & | 6.28 & | 4.26-4.28; | 3.90-3.93; | 3.87-3.89; | 3.41-3.49; | NA | NA |
| | | | | | 5.46; d | 7.66; d | 6.29; d | t | m | m | m | | |

TABLE G
Proton NMR of 2′-OMe (ara) nucleosides:

| | 2′- | | | | | | | | | H-5′ & |
|---|---|---|---|---|---|---|---|---|---|---|
| Ncleoside | OCH₃ | H2 | H8 | H5 | H6 | H-1′ | H-2′ | H-3′ | H-4′ | H-5″ |
| A | | | | | | | | | | |
| C | 3.15 | NA | NA | 5.69 & | 7.55 & | 6.12-6.13; d | 3.96-3.98; t | 3.74-3.76; | 3.65-3.68; | 3.58-3.52; m |
| | | | | 5.71; d | 7.56; d | | | qt | qt | |
| G | 3.14 | NA | 7.75; s | NA | NA | 6.10-6.11; m | 4.18-4.21; t | 3.90-3.91; t | 3.68-3.91; | 3.56-3.63; m |
| | | | | | | | | | qt | |
| U | 3.24 | NA | NA | 5.58 & | 7.63 & | 6.10-6.12; m | 4.00-4.02; t | 3.83-3.85; | 3.65-3.68; m | 3.59-3.62 & |
| | | | | 5.59; d | 7.64; d | | | qt | | 3.52-3.54; m |

TABLE H
Proton NMR of 5′-DMT-2′-OMe (Ara)-N-Protected) nucleosides:

| | 2′- | | | | | | | | | | H-5′ & | CH | CH₃ |
|---|---|---|---|---|---|---|---|---|---|---|---|---|---|
| Nucle- | ara- | DMT | | | | | | | | | H- | of | of |
| oside | OCH₃ | (O—CH₃)₂ | H2 | H8 | H5 | H6 | H-1′ | H-2′ | H-3′ | H-4′ | 5″ | ibu | ibu |
| (n- | | | | | | | | | | | | | |
| bz)A | | | | | | | | | | | | | |
| (n- | 3.22 | 3.79 & | NA | 7.75 | 7.57 & | 8.02 & | 6.42-6.43; | 4.26-4.28; | 4.13-4.14; | 4.10-4.11; | 3.37-3.43; | NA | NA |
| bz)C | | 3.80 | | | 7.58; d | 8.03; d | d | t | m | m | m | | |
| (n- | 3.03 | 3.70 | NA | 7.81 | NA | NA | 6.09 & | 4.46; | 4.17-4.18; | 3.76; m | 3.44-3.29; | 2.82-2.86; | 1.16-1.25; |
| ibu)G | | | | | | | 6.10 (d) | br | t | | m | qt | qt |
| U | 3.33 | 3.78 | NA | NA | 5.44 & | 7.65 & | 6.28 & | 4.26-4.28; | 3.90-3.93; | 3.87-3.89; | 3.41-3.49; | NA | NA |
| | | | | | 5.46; d | 7.66; d | 6.29; d | t | m | m | m | | |

Conformational Considerations: In oligonucleotides as well as nucleosides the furanose ring is puckered to relieve strain and can either adopt C^{2′}-endo or the C^{3′}-endo conformation. Normally in nucleosides or oligonucleotides there is rapid equilibrium at roomtemperature. In our present invention the sugars are expected to be locked in C^{2′}-endo conformation.

We claim:

1. A nucleoside comprising Ara-Omethyl as a component of its structure.

2. The nucleoside of claim 1 incorporating an exocyclic amine protecting group selected from the group consisting of N6,N6-dimethyl adenine, N6-benzoyladenine, N-1-methyladenine, 7-deazaadenine, 7-deaza-8-azaadenine, 3-deazaadenine, ethenoadenine, isoguanine, N1-methylguanine, 7-iodo-7-deazaguanine, 7-deaza-7-iodo adenine, 7-deaza-7-iodo-6-oxopurine, 5-iodo-5-methyl-7-deazaguanine, 7-deazaguanine substituted with —C≡C(CH₂)₁₋₈-pthlamide, 7-deaza-8-azaguanine, 8-methylguanine, 8-bromoguanine, 8-aminoguanine, hypoxanthine, 6-methoxypurine, 7-deaza-6-oxopurine, 6-oxopurine, 2-aminopurine, 2,6-diaminopurine, 8-bromopurine, 8-aminopurine, 8-alkylaminopurine, 8-alkylaminopurine, thymine, N-3 methyl thymine, 5-acroxymethylcytosine, 5-azacytosine, isocytosine, N-4(C₁-C₆)alkylcytosine, N-3(C₁-C₆)alkylcytidine, 5-propynylcytosine, 5-iodo-cytosine, 5-(C₁-C₆)alkylcytosine, 5-aryl(C₁-C₆)alkylcytosine, 5-trifluoromethylcytosine, 5-methylcytosine, ethenocytosine, cytosine and uracil substituted with —CH═CH—C(═O)NH(C₁-C₆)alkyl, cytosine and uracil substituted with —C≡C—CH₂-phthalimide, NH(C₁-C₆)alkyl, 4-thiouracil, 2-thiouracil, N³-thiobenzoylethyluracil, 5-propynyluracil, 5 Oacetoxymethyluracil, 5-fluorouracil, 5-chlorouracil, 5-bromouracil, 5-iodouracil, 4-thiouracil, N-3-(C₁-C₆) alkyluracil, 5-(3-aminoallyl)-uracil, 5-(C₁-C₆)alkyluracil, 5-aryl(C₁-C₆)alkyluracil, 5-trifluoro methyluracil, 4-triazolyl-5-methyluracil, 2-pyridone, 2-oxo-5-methylpyrimidine, 2-oxo-4-methylthio-5-methylpyrimidine, 2-thiocarbonyl-4-oxo-5-methylpyrimidine, and 4-oxo-5-methylpyrimidine.

3. The nucleoside of claim 2 further incorporating a 5′- or 3′-4,4′-dimethoxytrityl.

4. The nucleoside of claim 2 further incorporating any member of the group consisting of 5′- or 3′-4, 4′,4″-trimethoxytrityl.

5. The nucleoside of claim 2 further incorporating a phosphoramidite group.

6. The nucleoside of claim 5 where the phosphoramidite consists of cyanoethyl group as phosphate protecting group.

7. The nucleoside of claim 6 where the phosphoramidite consist of n,n-diisopropyl amino group.

8. The nucleoside of claim 2 further incorporating 5′- or 3′-4′-monomethoxytrityl.

9. The oligonucleotide synthesized using the nucleosides of claim 5, 6 or 7 as components.

10. The oligonucleotide of claim 9 further incorporating modified bases.

11. The oligonucleotide of the claim 10 designed to include an aptamer targeting a specified protein or peptide.

12. The oligonucleotide of the claim 11 designed to target telomerase, and telomerase binding ability known to result in a stable G-quadruplex.

13. The oligonucleotide of the claim 11 synthesized targeting a specified protein present in a virus.

14. The oligonucleotide of claim 13 wherein the targeted protein relates to the life cycle of a virus.

15. The oligonucleotide of the claim 11 synthesized to target a specified protein with significance as an antimetabolite in humans or animals.

16. The nucleoside of claim 1 synthsized for therapeutic use.
